(19) United States
(12) Patent Application Publication
(10) Pub. No.: US 2024/0018592 A1
(43) Pub. Date: Jan. 18, 2024

(54) METHODS OF ASSESSING THE THERAPEUTIC ACTIVITY OF AGENTS FOR THE TREATMENT OF IMMUNE DISORDERS

(71) Applicants: Technion Research & Development Foundation Limited, Haifa (IL); Rambam Med-Tech Ltd., Haifa (IL)

(72) Inventors: Shai S. SHEN-ORR, Haifa (IL); Yehuda CHOWERS, Tel-Aviv (IL); Ayelet ALPERT, Haifa (IL); Shiran GERASSY-VAINBERG, Tirat Carmel (IL); Erik FELDMAN, Haifa (IL)

(73) Assignees: Technion Research & Development Foundation Limited, Haifa (IL); Rambam Med-Tech Ltd., Haifa (IL)

(21) Appl. No.: 18/033,582

(22) PCT Filed: Oct. 26, 2021
(86) PCT No.: PCT/IL2021/051266

§ 371 Date: Apr. 25, 2023

Related U.S. Application Data
(60) Provisional application No. 63/105,393, filed on Oct. 26, 2020.

(51) Int. Cl. C12Q 1/6883; C12Q 1/6837

(52) U.S. Cl. CPC C12Q 1/6883; C12Q 1/6837; C12Q 2600/158; C12Q 2600/106

(57) ABSTRACT

A method of analyzing the therapeutic efficacy of a therapeutic agent for treating an immune-related disorder is disclosed. The method comprises contacting blood cells of the subject with the agent, measuring the expression of at least 20 genes in the blood cells of the subject; obtaining an immune age value based on the expression of the at least 20 genes, and comparing the immune age value with an immune age value of the subject calculated on the basis of expression of the at least 20 genes in blood cells of the subject, in the absence of the agent.

### RELATED APPLICATIONS

This application claims the benefit of priority of U.S. Provisional Patent Application No. 63/105,393 filed 26 Oct. 2020, the contents of which are incorporated herein by reference in their entirety.

### FIELD AND BACKGROUND OF THE INVENTION

The present invention, in some embodiments thereof, relates to methods of assessing the therapeutic efficacy of therapeutic agents for treating immune-related disorders.

Chronic diseases are a growing healthcare problem which lead to a steadily reduction in the populations' health span. Over the past decade it has become apparent that the aging immune system has a fundamental role in a variety of these diseases including cardiovascular disease, cancer, neurodegeneration, musculoskeletal conditions and many. This places the immune system as an “aging rheostat” from which we can learn mechanisms of disease, and against which we can intervene.

Epidemiological studies and clinical trials have indicated that many age-associated chronic diseases can be prevented, and even reversed, by lifestyle interventions including exercise, diet or drugs targeting different disease associated pathways. Most of these studies were context specific based on associations of the tested interventions with the relevant specific clinical and laboratory measurements (Fontana et al. 2009; PNAS).

It has recently been discovered that older adults change their immune composition gradually along an axis defined by immune cell abundances. Despite this conserved pattern of immune aging, older adults advance along this axis at different rates, resulting with a high heterogeneity in immune profiles. Using this axis, an IMM-AGE metric was developed that corresponds to the relative location of individuals along the immune-age axis and reflects the biological age of the immune system. This novel metric is associated with cardiovascular malfunctioning and predicts mortality risk beyond well-established risk factors such as age, gender, cardiovascular disease, etc. (Alpert et al., Nat Med, 2019 March; 25(3):487-495).

Additional background art includes WO 2019/215740.

### SUMMARY OF THE INVENTION

According to an aspect of the present invention there is provided a method of treating an immune related disorder in a subject in need thereof comprising:
(a) contacting blood cells of the subject with the agent;
(b) measuring the expression of at least 20 genes selected from the group consisting of ABLIM1, AFF3, BACH2, BCL11A, BIRC3, BLNK, BTLA, C11orf31, C6orf48, CCR6, CCR7, CD200, CD22, CD27, CD28, CDCA7L, CHMP7, CR2, DPP4, E2F5, EPHX2, FAIM3, FAM102A, FAM134B, FCRL1, FCRL2, HOOK1, HVCN1, IL6ST, IMPDH2, KIAA0748, LEF1, LRRN3, MYC, NELL2, NT5E, P2RX5, PAQR8, PLAG1, PTPRK, RCAN3, SCML1, SLC7A6, SNX9, STRBP, SUSD3, TCF4, TCF7, TCL1A, TCTN1, UXT, VPREB3 and ZNF101 in the blood cells of the subject;
(c) obtaining an immune age value based on the expression of the at least 20 genes;
(d) comparing the immune age value with an immune age value of the subject calculated on the basis of expression of the at least 20 genes in blood cells of the subject, in the absence of the agent, wherein a decrease in the immune age of the subject is indicative that the agent has therapeutic efficacy for treating the immune-related disorder; and
(e) administering the agent to the subject.

According to an aspect of the present invention there is provided a method of treating an immune related disorder in a subject in need thereof comprising:
(a) contacting blood cells of the subject with the agent;
(b) measuring the expression of each of the genes BACH2, BCL11A, CHMP7, DPP4 and LRRN3 in the blood cells of the subject;
(c) obtaining an immune age value based on the expression of the at least genes;
(d) comparing the immune age value with an immune age value of the subject calculated on the basis of expression of the genes in blood cells of the subject, in the absence of the agent, wherein a decrease in the immune age of the subject is indicative that the agent has therapeutic efficacy for treating the immune-related disorder; and
(e) administering the agent to the subject.

According to an aspect of the present invention there is provided a method of analyzing the therapeutic efficacy of a therapeutic agent for treating an immune-related disorder of a subject:
(a) contacting blood cells of the subject with the agent;
(b) measuring the expression of at least 20 genes selected from the group consisting of ABLIM1, AFF3, BACH2, BCL11A, BIRC3, BLNK, BTLA, C11orf31, C6orf48, CCR6, CCR7, CD200, CD22, CD27, CD28, CDCA7L, CHMP7, CR2, DPP4, E2F5, EPHX2, FAIM3, FAM102A, FAM134B, FCRL1, FCRL2, HOOK1, HVCN1, IL6ST, IMPDH2, KIAA0748, LEF1, LRRN3, MYC, NELL2, NT5E, P2RX5, PAQR8, PLAG1, PTPRK, RCAN3, SCML1, SLC7A6, SNX9, STRBP, SUSD3, TCF4, TCF7, TCL1A, TCTN1, UXT, VPREB3 and ZNF101 in the blood cells of the subject;
(c) obtaining an immune age value based on the expression of the at least 20 genes; and
(d) comparing the immune age value with an immune age value of the subject calculated on the basis of expression of the at least 20 genes in blood cells of the subject, in the absence of the agent, wherein a decrease in the immune age of the subject is indicative that the agent has therapeutic efficacy for treating the immune-related disorder of the subject.

According to an aspect of the present invention there is provided a method of analyzing the therapeutic efficacy of a therapeutic agent for treating an immune-related disorder of a subject:
(a) contacting blood cells of the subject with the agent;
(b) measuring the expression of each of the genes BACH2, BCL11A, CHMP7, DPP4 and LRRN3 in the blood cells of the subject;
(c) obtaining an immune age value based on the expression of the genes; and
(d) comparing the immune age value with an immune age value of the subject calculated on the basis of expression of the at genes in blood cells of the subject, in the absence of the agent, wherein a decrease in the immune age of the subject is indicative that the agent has therapeutic efficacy for treating the immune-related disorder of the subject.

According to embodiments of the invention, the immune age value is determined based on expression of no more than 150 genes.

According to embodiments of the invention, the immune age value is determined based on expression of no more than 60 genes.

According to an aspect of the present invention the method of treating an immune related disorder in a subject in need thereof comprising:
(a) contacting peripheral blood cells of the subject with the agent;
(b) measuring the frequency of at least 10 cells types selected from the group consisting of B cells, CD161⁻CD45RA⁺Tregs, CD161⁺ NK cells, CD28⁻CD8⁺T cells, CD57⁺CD8⁺T cells, CD57⁺ NK cells, effector CD8⁺T cells, effector memory CD4⁺T cells, effector memory CD8⁺T cells, HLADR⁻CD38⁺CD4⁺T cells, naïve CD4⁺T cells, naïve CD8⁺T cells, PD1⁺CD8⁺T cells, T cells, CXCR5⁺CD4⁺T cells, CXCR5⁺CD8⁺T cells, Th17 CXCR5⁻ cells, Tregs in a peripheral blood cell sample of the subject;
(c) obtaining an immune age value based on the frequency of the at least 10 cell types in the peripheral blood cells;
(d) comparing the immune age with an immune age of the subject calculated on the basis of the frequency of the at least 10 cell types of the subject in the absence of the agent, wherein a decrease in the immune age of the subject is indicative that the agent has therapeutic efficacy for treating the immune-related disorder; and
(e) administering the agent to the subject.

According to an aspect of the present invention the method of treating an immune related disorder in a subject in need thereof comprising:
(a) contacting peripheral blood cells of the subject with the agent;
(b) measuring the frequency of each of the cell types: CD161⁺NK cells, CD57⁺CD8⁺T cells, CD57⁺ NK cells and T cells in a peripheral blood cell sample of the subject;
(c) obtaining an immune age value based on the frequency of the cell types in the peripheral blood cells;
(d) comparing the immune age with an immune age of the subject calculated on the basis of the frequency of the cell types of the subject in the absence of the agent, wherein a decrease in the immune age of the subject is indicative that the agent has therapeutic efficacy for treating the immune-related disorder; and
(e) administering the agent to the subject.

According to an aspect of the present invention the method of analyzing the therapeutic efficacy of a therapeutic agent for treating an immune-related disorder of a subject:
(a) contacting peripheral blood cells of the subject with the agent;
(b) measuring the frequency of at least 10 cell types selected from the group consisting of B cells, CD161⁻CD45RA⁺Tregs, CD161⁺ NK cells, CD28⁻CD8⁺T cells, CD57⁺CD8⁺T cells, CD57⁺ NK cells, effector CD8⁺T cells, effector memory CD4⁺T cells, effector memory CD8⁺T cells, HLADR⁻CD38⁺CD4⁺T cells, naïve CD4⁺T cells, naïve CD8⁺T cells, PD1⁺CD8⁺T cells, T cells, CXCR5⁺CD4⁺T cells, CXCR5⁺CD8⁺T cells, Th17 CXCR5⁻ cells, Tregs in the peripheral blood cells of the subject;
(c) obtaining an immune age value based on the frequency of the at least 10 cell types in the peripheral blood cells; and
(d) comparing the immune age with an immune age of the subject calculated on the basis of the frequency of the at least 10 cell types of the subject, in the absence of the agent, wherein a decrease in the immune age of the subject is indicative that the agent has therapeutic efficacy for treating the immune-related disorder of the subject.

According to an aspect of the present invention the method of analyzing the therapeutic efficacy of a therapeutic agent for treating an immune-related disorder of a subject:
(a) contacting peripheral blood cells of the subject with the agent;
(b) measuring the frequency of the cell types CD161⁺NK cells, CD57⁺CD8⁺T cells, CD57⁺ NK cells and T cells in the peripheral blood cells of the subject;
(c) obtaining an immune age value based on the frequency of the cell types in the peripheral blood cells; and
(d) comparing the immune age with an immune age of the subject calculated on the basis of the frequency of the cell types of the subject, in the absence of the agent, wherein a decrease in the immune age of the subject is indicative that the agent has therapeutic efficacy for treating the immune-related disorder of the subject.

According to embodiments of the invention, the method further comprises measuring the expression of at least one additional gene selected from the group consisting of AGPAT4, AKAP2, APBB1, ASCL2, C1orf21, C20orf3, CHST12, CST7, CTSW, EEF1B2, ELLS, FAM113B, FAM129C, FCER2, FCGBP, FCRL6, FGFBP2, FLT3LG, GAL3ST4, GPR56, GPR68, GZMH, HOXC4, ID3, LLGL2, LTB, MMP23A, MOBKL2B, MXRA7, MYO6, NKG7, NOG, NOSIP, PCYOX1L, PLEKHF1, PMEPA1, RNF157, RPL12, RPL24, RPS10, RPS13, RPS5, SAP30, SESN2, SYTL3, TBX21, TGFBR3, TNFRSF25, TSPAN13, TTC28, YPEL1, ZNF154, ZNF563, ZNF772, ZSCAN18, C2orf89, PATL2, TTC38, PRR5L, SGK223, NCRNA00287, IGHM, HLA-DOA and IGHV5-78, wherein the immune age value is based on the expression of the at least one additional gene and the expression of the at least twenty genes.

According to embodiments of the invention, the contacting is effected in vivo.

According to embodiments of the invention, the measuring is effected ex vivo.

According to embodiments of the invention, the measuring is effected no earlier than 1 week following the contacting.

According to embodiments of the invention, the measuring is effected no more than 14 weeks following the contacting.

According to embodiments of the invention, the blood cells comprise peripheral blood cells.

According to embodiments of the invention, the immune-related disorder is a chronic immune-related disorder.

According to embodiments of the invention, the immune-related disorder is selected from the group consisting of inflammatory bowel disease, rheumatoid arthritis, ankylosing spondylitis, psoriasis, psoriatic arthritis and Behçet's disease.

According to embodiments of the invention, the inflammatory bowel disease is Crohn's disease (CD) or ulcerative colitis (UC).

According to embodiments of the invention, the agent is an agent that reduces the amount or activity of tumor necrosis factor alpha (TNF-α).

According to embodiments of the invention, the agent is an inhibitory antibody that specifically binds to TNF-α.

According to embodiments of the invention, the antibody is selected from the group consisting of infliximab, adalimumab, certolizumab pegol and golimumab.

According to embodiments of the invention, the agent is selected from the group consisting of etanercept, thalidomide, lenalidomide, pomalidomide, pentoxifulline, bupriopion, R)-DOI, TCB-2, LSD and LA-SS-Az.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-E. Immune age is modulated by anti-TNF treatment in Crohn's disease patients, highly associated with disease specific molecular immune metric and significantly reflects patients' response potential. (A) External public based ‘response axis’ which defines a transition from active to in-active disease states in IBD patients defined by PCA based on cell centered differential expressed genes between active (red) and inactive (cyan) UC (circle) and CD (triangles) patient groups. (B) Projection of in-house responding patients on the ‘response axis’. (C) Scatterplot presenting high correlation between the IBD molecular response axis and the immune age score. (D) comparison of immune age score in responding patients pre-treatment (V1) and 2 w (V2) and 14 week (V3) post first treatment. Significance was determined by Wilcoxon paired test. (E) Boxplot comparing the change in Immune age at 14 week post treatment relative to baseline, between responding (cyan) and non-responding (red) patients. Statistical significance was calculated by Wilcoxon test.

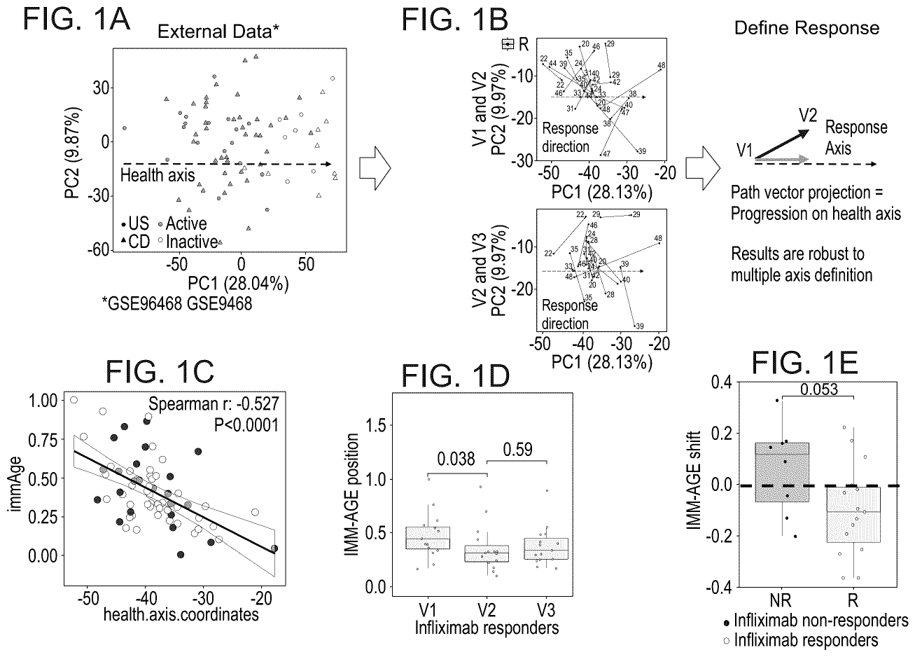

### DESCRIPTION OF SPECIFIC EMBODIMENTS OF THE INVENTION

The present invention, in some embodiments thereof, relates to methods of assessing the therapeutic efficacy of therapeutic agents for treating immune-related disorders.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

The human immune system changes with age, ultimately leading to a clinically evident, profound deterioration resulting in high morbidity and mortality rates attributed to infectious and chronic diseases. At the cellular level, population-based cross-sectional studies have shown that many immune components change with age, spanning both the innate and adaptive arms of the immune system, and involving changes in cellular frequencies and altered functional capacity. Concomitant with the overall down-regulation of immune responsiveness with aging, a moderate rise in circulating inflammatory mediators, commonly termed ‘inflammaging’, is often observed.

The present inventors previously showed that high-individual variability exists in the rates of changes of immune cellular frequencies, that was dictated by their baseline values. This novel metric was shown to be associated with cardiovascular malfunctioning and predicted mortality risk beyond well established risk factors such as age, gender, cardiovascular disease, etc (Alpert et al., Nat Med, 2019 March; 25(3):487-495).

The present inventors now propose to identify ways to modulate individuals' immune age by shifting it back to a younger phenotype in order to enhance an individual health state and extend life span. By using the immune age measurement as a multi-dimensional high resolution tool for prognostic monitoring that enables positioning individuals at specific locations along the immune age trajectory at baseline, and to follow their dynamics as a result of different interventions or therapies, the present inventors show that it is possibly to accurately assess treatment efficacy and therefore enhance treatment adaptation, at an early stage. Owing to its predictive nature, the manipulation of the immune age can be applied to prevent age-related disease development by early identification of patients at risk and using specific interventions that shifts back their immune age.

Thus, according to a first aspect of the present invention, there is provided a method of analyzing the therapeutic efficacy of a therapeutic agent for treating an immune-related disorder of a subject:
(a) contacting peripheral blood cells of the subject with the agent;
(b) measuring the frequency of the cell types CD161⁺NK cells, CD57⁺CD8⁺T cells, CD57⁺ NK cells and T cells in the peripheral blood cells of the subject;
(c) obtaining an immune age value based on the frequency of the cell types in the peripheral blood cells; and
(d) comparing the immune age with an immune age of the subject calculated on the basis of the frequency of the cell types of the subject, in the absence of the agent, wherein a decrease in the immune age of the subject is indicative that the agent has therapeutic efficacy for treating the immune-related disorder of the subject.

According to another aspect of the present invention, there is provided a method of analyzing the therapeutic efficacy of a therapeutic agent for treating an immune-related disorder of a subject:
(a) contacting blood cells of the subject with the agent;
(b) measuring the frequency of at least 10 cell types selected from the group consisting of B cells, CD161⁻CD45RA⁺Tregs, CD161⁺ NK cells, CD28⁻CD8⁺T cells, CD57⁺CD8⁺T cells, CD57⁺ NK cells, effector CD8⁺T cells, effector memory CD4⁺T cells, effector memory CD8⁺T cells, HLADR⁻CD38⁺CD4⁺T cells, naïve CD4⁺T cells, naïve CD8⁺T cells, PD1⁺CD8⁺T cells, T cells, CXCR5⁺CD4⁺T cells, CXCR5⁺CD8⁺T cells, Th17 CXCR5⁻ cells, Tregs in the blood cells of the subject;
(c) obtaining an immune age value based on the frequency of the at least 10 cell types in the blood cells; and
(d) comparing the immune age with an immune age of the subject calculated on the basis of the frequency of the at least 10 cell types of the subject, in the absence of the agent, wherein a decrease in the immune age of the subject is indicative that the agent has therapeutic efficacy for treating the immune-related disorder.

The method is typically an ex vivo or in vivo method for selecting an agent for treating an immune related disorder, for a particular subject (i.e. personalized medicine).

The subject who is suffering from the immune-related disorder is a mammal. In some embodiments, the subject is a human. In some embodiments, the subject is a veterinary animal. In some embodiments, the subject is elderly. In some embodiments, elderly is at least 30, 35, 40, 45, 55, 60, 65, 70, 76, 80, 85 or 90 years old. In some embodiments, the subject s at least 40 years old. In some embodiments, the subject is at least 60 years old.

The blood cells comprise peripheral blood cells. In a particular embodiment, a peripheral blood sample is taken from the subject. In another embodiment, a bone marrow sample is retrieved and immune cells are isolated.

Contacting of the blood cells with the candidate agent may be carried out in vivo or ex vivo. Typically, the measurement of the cell frequencies is carried out at least 1 day, 2 days, 3, days, 1 week, 2 weeks, 4 weeks, 7 weeks, 10 weeks, 12 weeks, 14 weeks following the contacting.

When the contacting is carried out in vivo, the candidate agent is provided to the subject and following the allotted length of time, blood cells are retrieved from the subject.

When the contacting is carried out ex vivo, blood cells are retrieved from the subject, and the candidate agent is contacted with the blood cells.

The frequency of particular cell types (following contact with the candidate agent) is then measured and compared with the frequency of those cell types in the absence of the candidate therapeutic agent (e.g. prior to contacting with the candidate agent).

The candidate therapeutic agents are selected according to the particular immune-related disorder from which the subject is suffering.

Exemplary candidate agents that may be tested include those that reduce the amount or activity of tumor necrosis factor alpha (TNF-α).

In one embodiment, the candidate agents are inhibitory antibodies that specifically bind to TNF-α.

Examples of such antibodies include but are not limited to infliximab, adalimumab, certolizumab pegol and golimumab.

Additional examples of agents that reduce TNF-α include, but are not limited to etanercept, thalidomide, lenalidomide, pomalidomide, pentoxifulline, bupriopion, R)-DOI, TCB-2, LSD and LA-SS-Az.

The cell types which are measured according to this aspect of the present invention are typically immune cells.

As used herein, “immune cell” refers to any cell of the immune system. In some embodiments, the immune cell is a hematopoietic cell. In some embodiments, the immune cell population is selected from the group consisting of: naive CD8+ T cells, effector CD8+ T cells, CD28− CD8+ T cells, B cells, CXCR5+ CD4+ T cells, CD161− CD45RA+ T regulator cells, naive CD4+ T cells, CXCR5+ CD8+ T cells, HLADR− CD38+ CD4+ T cells, Th17 CXCR5− CD4+ T cells, T cells, CD85j+ CD8+ T cells, CD57+ CD8+ T cells, Th2 non-TFH CD4+ T cells, PD1+ CD8+ T cells, effector memory CD4+ T cells, CD27+ CD8+ T cells, lymphocytes, central memory CD4+ T cells, natural killer (NK) cells, monocytes, Th1 TFH CD4+ T cells, CD8+ T cells, CXCR3-CCR6− CXCR5+×CD8+ T cells, Th2 TFH, CD4+ T cells, plasmablasts, and CD94+ NK cells. In some embodiments, the immune cell population is a population with an asymptotic and/or linear trajectory. In some embodiments, the immune cell population is selected from the group consisting of: naive CD8+ T cells, effector CD8+ T cells, CD28− CD8+ T cells, B cells, CXCR5+ CD4+ T cells, CD161−CD45RA+ T regulator cells, naive CD4⁺T cells, CXCR5+ CD8+ cells, HLADR-CD38+ CD4+ T cells, Th17 CXCR5− CD4+ T cells, T cells, CD85j+ CD8+ T cells, CD57+ CD8+ T cells, Th2 non-TFH CD4+ T cells, PD1+CD8+ T cells, and effector memory CD4⁺ T cells. In some embodiments, the immune cell population is a population with an asymptotic trajectory. In some embodiments, the population is selected from the group consisting of: naive CD8+ T cells, effector CD8+ T cells, CD28− CD8+ T cells, B cells, CXCR5+ CD4+ cells, CD161− CD45RA+ T regulator cells, naive CD4+ T cells, CXCR5+ CD8+ T cells, HLADR− CD38+ CD4+ T cells, Th17 CXCR5− CD4+ T cells, and T cells. In some embodiments, the population with asymptotic trajectory is selected from the group consisting of: naive CD8+ T cells, effector CD8+ T cells, CD28− CD8+ cells, B cells, CXCR5+×CD4⁺T cells, CD161− CD45RA+ T regulator cells, naive CD4+ T cells, CXCR5+ CD8+ T cells, HLADR− CD38+ CD4+ T cells, Th17 CXCR5− CD4+ T cells, and T cells. In some embodiments, the population with linear trajectory is selected from the group consisting of: CD85j+ CD8+ T cells, CD57+ CD8+ T cells, Th2 non-TFH CD4+ T cells, PD1+ CD8+ T cells, and effector memory CD4+ T cells. In some embodiments, the population with fluctuating trajectory is selected from the group consisting of: CD27+ CD8+ T cells, lymphocytes, central memory CD4+ T cells, natural killer (NK) cells, monocytes, Th1 TFH CD4+ T cells, CD8+ T cells, CXCR3− CCR6− CXCR5+ CD8+ T cells, Th2 TETI CD4+ T cells, plasmablasts, and CD94+ NK cells.

In some embodiments, the frequency of a particular cell type is measured by expression of at least one epitope that identifies the population. In some embodiments, the expression is surface expression, in some embodiments, the expression is intracellular expression. In some embodiments, the epitope is an immune cell marker. Immune cell markers are well known in the art and any marker or markers than can uniquely and/or unambiguously identify the population may be used. In some embodiments, measuring a population's abundance comprises measuring abundance of at least one epitope indicative of the immune cell population. In some embodiments, the measuring is on a single cell level. In some embodiments, the measuring comprises extracting cells from the blood sample. In some embodiments, the measuring comprises contacting the cells blood sample with an agent that binds to at least one epitope that is indicative of and/or identifies the population. In some embodiments, the epitope is an extracellular epitope. In some embodiments, the epitope is an intracellular epitope. In some embodiments, the epitope is a protein. In some embodiments, the protein is a surface protein. In some embodiments, the agent is an antibody to the epitope. In some embodiments, the anent is conjugated to a detectable moiety and the measuring comprises measuring the moiety. In some embodiments, the measuring comprises immunodetection. In some embodiments, the immunodetection is flow cytometry. In some embodiments, the flow cytometry is fluorescence activated cell sorting (FACS). In some embodiments, the immunodetection is single-cell mass cytometry analysis (CyTOF). In some embodiments, the measuring comprises CyTOF. In some embodiments, a population is gated based on expression of at least one indicative epitope. In some embodiments, more than one population are gated in the same measuring and relative abundance is measured. In some embodiments, the immunodetection is immunostaining. In some embodiments, the detectable moiety is a fluorescent moiety. In some embodiments, the measuring comprises cell counting. Any methods of population detection, such as but not limited as are described herein, may be employed for the methods of the invention. Examples of antibodies that can be used for measuring can be found in Alpert et al., 2019, Nature Medicine, 25: 387-495, herein incorporated by reference in its entirety.

Particular antibodies and particular detectable moieties that can be used in order to pleasure the number of T cells—for example by flow cytometry—are listed herein below in Table 1.

TABLE 1

| epitope | conjugate | Manufacturer | Catalog # | Clone ID |
|---|---|---|---|---|
| CD85j | FITC | BD Biosciences | 623775 | GHI/75 |
| CD28 | PE | BD Biosciences | 623775 | L293 |
| CD4 | PERCP-Cy5.5 | BD Biosciences | 623775 | SK3 |
| CD45RA | PE-Cy7 | BD Biosciences | 623775 | HI100 |
| CD27 | APC | BD Biosciences | 623775 | L128 |
| CD8 | APC-H7 | BD Biosciences | 623775 | SK1 |
| CD3 | V450 | BD Biosciences | 623775 | UCHT1 |

Antibodies and particular detectable moieties that can be used in order to measure the number of NK/NKT cells—for example by flow cytometry, are listed herein below in Table 2.

TABLE 2

| epitope | conjugate | Manufacturer | Catalog # | Clone ID |
|---|---|---|---|---|
| CD94 | FITC | BD Biosciences | 623775 | HP-3D9 |
| CD314 | PE | BD Biosciences | 623775 | 1D11 |
| HLADR | PERCP-Cy5.5 | BD Biosciences | 623775 | L243 |
| CD16 | PE-Cy7 | BD Biosciences | 623775 | 3G8 |
| CD56 | APC | BD Biosciences | 623775 | NCAM16.2 |
| CD8 | APC-H7 | BD Biosciences | 623775 | SK1 |
| CD3 | V450 | BD Biosciences | 623775 | UCHT1 |

Antibodies and particular detectable moieties that can be used in order to measure the number of B cells—for example by flow cytometry, are listed herein below in Table 3.

TABLE 3

| epitope | conjugate | Manufacturer | Catalog # | Clone ID |
|---|---|---|---|---|
| IgD | FITC | BD Biosciences | 623775 | IA6-2 |
| CD24 | PE | BD Biosciences | 623775 | ML5 |
| CD19 | PERCP-Cy5.5 | BD Biosciences | 623775 | SJ25C1 |
| CD38 | PE-Cy7 | BD Biosciences | 623775 | HB-7 |
| CD27 | APC | BD Biosciences | 623775 | L128 |
| CD20 | APC-H7 | BD Biosciences | 623775 | 2H7 |
| CD3 | V450 | BD Biosciences | 623775 | UCHT1 |

Antibodies and particular detectable moieties that can be used in order to measure the number of T reg cells—for example by flow cytometry, are listed herein below in Table 4.

TABLE 4

| epitope | conjugate | Manufacturer | Catalog # | Clone ID |
|---|---|---|---|---|
| CD161 | FITC | BD Biosciences | 623775 | DX12 |
| CD25 | PE | BD Biosciences | 623775 | 2A3 |
| CD4 | PERCP-Cy5.5 | BD Biosciences | 623775 | SK3 |
| CD45RA | PE-Cy7 | BD Biosciences | 623775 | HI100 |
| CD127 | APC | BD Biosciences | 623775 | HIL-7R-M21 |
| CD8 | APC-H7 | BD Biosciences | 623775 | SK1 |
| CD3 | V450 | BD Biosciences | 623775 | UCHT1 |

Antibodies and particular detectable moieties that can be used in order to measure the number of FMO CXCR3 cells—for example by flow cytometry, are listed herein below in Table 5.

TABLE 5

| epitope | conjugate | Manufacturer | Catalog # | Clone ID |
|---|---|---|---|---|
| CD56 | FITC | BD Biosciences | 623775 | NCAM16.2 |
| CD16 | FITC | BD Biosciences | 623775 | 3G8 |
| CD4 | PERCP-Cy5.5 | BD Biosciences | 623775 | SK3 |
| CD33 | PE-Cy7 | BD Biosciences | 623775 | P67.6 |
| CD19 | APC | BD Biosciences | 623775 | SJ25C1 |
| CD8 | APC-H7 | BD Biosciences | 623775 | SK1 |
| CD3 | V450 | BD Biosciences | 623775 | UCHT1 |

Antibodies and particular detectable moieties that can be used in order to measure the number of CXCR3⁺ cells—for example by flow cytometry, are listed herein below in Table 6.

TABLE 6

| epitope | conjugate | Manufacturer | Catalog # | Clone ID |
|---|---|---|---|---|
| CD56 | FITC | BD Biosciences | 623775 | NCAM16.2 |
| CD16 | FITC | BD Biosciences | 623775 | 3G8 |
| CXCR3 | PE | BD Biosciences | 623775 | 1C6/CXCR3 |
| CD4 | PERCP-Cy5.5 | BD Biosciences | 623775 | SK3 |
| CD33 | PE-Cy7 | BD Biosciences | 623775 | P67.6 |
| CD19 | APC | BD Biosciences | 623775 | SJ25C1 |
| CD9 | APC-H7 | BD Biosciences | 623775 | SK1 |
| CD3 | V450 | BD Biosciences | 623775 | UCHT1 |

Antibodies and particular detectable moieties that can be used in order to measure the number of activated T cells—for example by flow cytometry—are listed herein below in Table 7.

TABLE 7

| epitope | conjugate | Manufacturer | Catalog # | Clone ID |
|---|---|---|---|---|
| TCRgd | FITC | BD Biosciences | 623775 | B1 |
| PD1 | PE | BD Biosciences | 623775 | EH12.1 |
| CD4 | PERCP-Cy5.5 | BD Biosciences | 623775 | SK3 |
| CD38 | PE-Cy7 | BD Biosciences | 623775 | HB-7 |
| HLADR | APC | BD Biosciences | 623775 | L243 |
| CD8 | APC-H7 | BD Biosciences | 623775 | SK1 |
| CD3 | V450 | BD Biosciences | 623775 | UCHT1 |

Additional antibodies that may be used to determine cell type are summarized in Table 8, herein below:

TABLE 8

| Metal | | | | |
|---|---|---|---|---|
| label | Specificity | Source | Catalog Number | Clone |
| 113In | CD57 | Biolegend | 322325 | HCD57 |
| 115In | live/dead | Macrocyclics | B-272 | Maleimide- |
| | | | | DOTA |
| 142Nd | CD19 | Southern Biotech | 363002 | SJ25C1 |
| 143Nd | CD4 | Biolegend | 344602 | SK3 |
| 144Nd | CD8 | Biolegend | 344702 | SK1 |
| 146Nd | IgD | Biolegend | 348202 | IA6-2 |
| 147Sm | CD85j | R&D Systems | MAB20172 | 292319 |
| 148Nd | CD11c | Biolegend | 337202 | Bu15 |
| 149Sm | CD16 | Biolegend | 302002 | 3G8 |
| 150Nd | CD3 | Biolegend | 300402 | UCHT1 |
| 151Eu | CD38 | Biolegend | 356602 | HB-7 |
| 152Sm | CD27 | BD | custom order | L128 |
| | | | (carrier-free) | |
| 153Eu | CD11b | Biolegend | 301302 | ICRF44 |
| 154Sm | CD14 | Biolegend | 301802 | M5E2 |
| 155Gd | CCR6 | BD | 559560 | 11A9 |
| 156Gd | CD94 | BD | 555887 | HP-3D9 |
| 157Gd | CD86 | Biolegend | 305402 | IT2.2 |
| 158Gd | CXCR5 | BD | 552032 | RF8B2 |
| 159Tb | CXCR3 | Biolegend | 353702 | G025H7 |
| 160Gd | CCR7 | R&D Systems | MAB197 | 150503 |
| 162Dy | CD45RA | Biolegend | 304102 | HI100 |
| 164Dy | CD20 | Biolegend | 302302 | 2H7 |
| 165Ho | CD127 | Biolegend | 351302 | A019D5 |
| 166Er | CD33 | Biolegend | 366602 | P67.6 |
| 167Er | CD28 | BD | 340975 | L293 |
| 168Er | CD24 | Biolegend | 311102 | ML5 |
| 169Tm | ICOS | BD | 557801 | DX29 |
| 170Er | CD161 | BD | 556079 | DX12 |
| 171Yb | TCRgd | Biolegend | 331202 | B1 |
| 172Yb | PD-1 | BD | 562138 | EH12.1 |
| 173Yb | CD123 | BD | 555642 | 9F5 |
| 174Yb | CD56 | BD | 559043 | NCAM16.2 |
| 175Lu | HLA-DR | BD | 556642 | G46-6 |
| 176Yb | CD25 | Biolegend | 356102 | M-A251 |

According to a particular embodiment, at least 4 cell types are identified (and used to calculate immune age).

According to a particular embodiment, at least 5 cell types are identified (and used to calculate immune age).

According to a particular embodiment, at least 6 cell types are identified (and used to calculate e age).

According to a particular embodiment, at least 7 cell types are identified (and used to calculate immune age).

According to a particular embodiment, at least 8 cell types are identified (and used to calculate immune age).

According to a particular embodiment, at least 9 cell types are identified (and used to calculate immune age).

According to a particular embodiment, at least 10 cell types are identified (and used to calculate immune age).

According to a particular embodiment, at least 20 cell s are identified (and used calculate immune age).

According to a particular embodiment, at least 30 cell types are identified (and used to calculate immune age).

According to a particular embodiment, at least 40 cell types are identified (and used to calculate age).

According to a particular embodiment, at least 50 cell types are identified (and used to calculate immune age).

In still another embodiment, no more than 5 different immune cell types are measured, no more than 10 different immune cell types are measured, no more than 15 different immune cell types are measured, no more than 20 different immune cell types are measured, no more than 25 different immune cell types are measured, no more than 30 different immune cell types are measured, no more than 35 different immune cell types are measured, no more than 40 different immune cell types are measured, no more than 45 different immune cell types are measured, no more than 50 different immune cell types are measured.

According to a particular embodiment, each of the following 4 cell types are measured: CD161⁺NK cells, CD57⁺CD8⁺T cells, CD57⁺ NK cells and T cells.

According to another embodiment, each of the following cell types are measured: Effector CD8⁺T cells, Effector memory CD4⁺T cells, Effector memory CD8⁺T cells, Naive CD4⁺T cells, Naive CD8₊T cells and T cells.

According to another embodiment, each of the following cell types are measured: CD28⁻CD8⁺T cells, CD57⁺CD8⁺T cells and T cells.

According to another embodiment, each of the following cell types are measured: CD28⁻CD8⁺T cells, Effector CD8⁺T cells, Effector memory CD4⁺T cells, Effector memory CD8⁺T cells, Naive CD4⁺T cells, naive CD8⁺T cells and T cells.

According to another embodiment, each of the following cell types are measured: CD28⁻CD8⁺T cells, CD57⁺CD8⁺T cells, T cells and regulatory T cells.

According to another embodiment, each of the following cell types are measured: CD57⁺CD8⁺T cells, CD57⁺NK cells, Effector CD8⁺T cells, Effector Memory CD4⁺T cells, Effector memory CD8⁺T cells, Naive CD4⁺T cells, Naive CD8⁺T cells and T cells.

According to another embodiment, each of the following cell types are measured: CD28⁻CD8⁺T cells, Effector CD8⁺T cells, Effector memory CD4⁺T cells, Effector memory CD8⁺T cells, naive CD4⁺T cells, naive CD8⁺T cells, T cells and regulatory T cells.

According to still another embodiment, each of the following cell types are measured: B cells, CD161⁺NK cells, CD57⁺CD8⁺T cells, CD57⁺NK cells, T cells.

The term “immune age” refers to the approximate age of a subject's immune system. This may be an absolute measurement or a relative measurement. The immune age is an artificial score based on the frequency of the particular cell types (or expression level of particular genes, as described herein below). In one embodiment the immune age increases with the frequency of the particular cell types. Thus, in this embodiment the higher the immune age of the subject, the more deleterious. In another embodiment, the immune age decreases with the frequency of the particular cell types. Thus, in this embodiment, the lower the immune age of the subject, the more deleterious.

Further details of establishing immune age are provided in WO2019215740, the contents of which are incorporated herein by reference.

In some embodiments, all the cell types described herein are measured. In some embodiments, each cell population (i.e. cell type) is measured simultaneously (i.e. in the same sample, or the same sample aliquot). In some embodiments, the cell populations are measured separately (e.g. from different blood samples, or from the same blood sample, but a different aliquot thereof). In some embodiments, total cell numbers are measured. In some embodiments, relative abundance is measured. In some embodiments, relative abundance is frequency, in some embodiments, frequency is relative frequency. In some embodiments, the percentage of each population in the blood sample is calculated. In some embodiments, measurements are at a single time point. In some embodiments, measurements are at more than one time point. In some embodiments, measurements are at least 1, 2, 3, 4, 5, 6, or 7 time points. Each possibility represents a separate embodiment of the invention. In some embodiments, the time points are separated by at least 1, 2, 3, 4, 5, 6, 8, 9, 10, 11 or 12 months. Each possibility represents a separate embodiment of the invention.

In some embodiments, the measuring is in blood of the subject. In some embodiments, the blood is a blood sample. In some embodiments, the blood is peripheral blood. In some embodiments, the relative abundance in peripheral blood is measured. In some embodiments, the measuring is performed ex vivo. In some embodiments, the measuring is performed in vitro. In some embodiments, the sample is a routine blood sample. In some embodiments, cells are isolated from the blood sample. In some embodiments, the relative abundance is measured in the blood. In some embodiments, nom-immune cells are removed before the measuring. In some embodiments, the non-immune cells are blood cells. In some embodiments, the blood cells are selected from red blood cells and platelets. In some embodiments, non-immune cells are left in the sample, but not included in the measuring. In some embodiments, the non-immune cells are gated out of the measuring.

Once a score is obtained based on the frequency of the particular cell types (i.e. the immune age) in the presence of the candidate agent, it is compared to the score obtained based on the frequency of those cell types in the absence of the candidate agent. If the candidate agent is contacted ex vivo with the blood cells of the subject, the score in the absence of the candidate agent may be ascertained at the same time as the score is determined in the presence of the candidate agent (i.e. using a different sample aliquot). Alternatively, if the candidate agent is contacted in vivo with the blood cells of the subject, the score in the absence of the candidate agent is ascertained at a previous time point (preferably no more than 1 week, 1 month or at most 6 months from the time the candidate agent is administered). In one embodiment, a panel of optional candidate agents are analysed and compared. The candidate agent that brings the immune age most close to the immune age of a healthy subject, (or most close to the immune age of the test subject at a less progressive stage of the disease) is typically selected as the most beneficial for treating the subject.

A database may be used which includes datasets of cell frequencies (or gene expression data, as further described below) from healthy subjects which can be used in to determine the immune age of the subject. Alternatively (and/or additionally), a database may be venerated which includes datasets of cell frequencies (or gene expression data, as further described below) from the subject suffering from the disease, at different time points along the course of the disease (e.g. every 6 months or every year). In one embodiment, the database includes datasets of cell frequencies (or gene expression data, as further described below) from the subject suffering from an active form of the disease and at a time when the subject is in remission.

It will be appreciated that instead of analysing cell frequencies (or as well as present inventors also contemplate analysing expression data of particular genes to obtain an immune age score. Thus, the method is carried out as described herein above, but instead of measuring cell frequencies, gene expression levels are measured.

Thus, according to another aspect of the present invention there is provided a method of analyzing the therapeutic efficacy of a therapeutic agent for treating an immune-related disorder of a subject:
(a) contacting blood cells of the subject with the agent;
(b) measuring the expression of at least 20 genes selected from the group consisting of ABLIM1, AFF3, BACH2, BCL11A, BIRC3, BLNK, BTLA, C11orf31, C6orf48, CCR6, CCR7, CD200, CD22, CD27, CD28, CDCA7L, CHMP7, CR2, DPP4, E2F5, EPHX2, FAIM3, FAM102A, FAM134B, FCRL1, FCRL2, HOOK1, HVCN1, IL6ST, IMPDH2, KIAA0748, LEF1, LRRN3, MYC, NELL2, NT5E, P2RX5, PAQR8, PLAG1, PTPRK, RCAN3, SCML1, SLC7A6, SNX9, STRBP, SUSD3, TCF4, TCF7, TCL1A, TCTN1, UXT, VPREB3 and ZNF101 in the blood cells of the subject;
(c) obtaining an immune age value based on the expression of the at least 20 genes; and
(d) comparing the immune age value with an immune age value of the subject calculated on the basis of expression of the at least 20 genes in blood cells of the subject, in the absence of the agent, wherein a decrease in the immune age of the subject is indicative that the agent has therapeutic efficacy for treating the immune-related disorder of the subject.

Table 9 herein below provides the direction the gene is regulated as immune age increases. Plus signifies that the expression increases as immune age increases (i.e. more detrimental to the subject), whereas minus signifies that the expression decreases as immune age increases (Thus, for example the expression of AGPAT4 increases as immune age increases and the expression of AKAP2 decreases as immune age increases).

TABLE 9

| | direction along |
|---|---|
| gene symbol | IMM-AGE axis |
| AGPAT4 | +1 |
| AKAP2 | −1 |
| APBB1 | −1 |
| ASCL2 | +1 |
| C1orf21 | +1 |
| C20orf3 | +1 |
| CHST12 | +1 |
| CST7 | +1 |
| CTSW | +1 |
| EEF1B2 | −1 |
| ELL3 | −1 |
| FAM113B | −1 |
| FAM129C | −1 |
| FCER2 | −1 |
| FCGBP | −1 |
| FCRL6 | +1 |
| FGFBP2 | +1 |
| FLT3LG | −1 |
| GAL3ST4 | −1 |
| GPR56 | +1 |
| GPR68 | +1 |
| GZMH | +1 |
| HOXC4 | +1 |
| ID3 | −1 |
| LLGL2 | +1 |
| LTB | −1 |
| MMP23A | +1 |
| MOBKL2B | −1 |
| MXRA7 | +1 |
| MYO6 | +1 |
| NKG7 | +1 |
| NOG | −1 |
| NOSIP | −1 |
| PCYOX1L | −1 |
| PLEKHF1 | +1 |
| PMEPA1 | −1 |
| RNF157 | −1 |
| RPL12 | −1 |
| RPL24 | −1 |
| RPS10 | −1 |
| RPS13 | −1 |
| RPS5 | −1 |
| SAP30 | +1 |
| SESN2 | +1 |
| SYTL3 | +1 |
| TBX21 | +1 |
| TGFBR3 | +1 |
| TNFRSF25 | −1 |
| TSPAN13 | −1 |
| TTC28 | −1 |
| YPEL1 | +1 |
| ZNF154 | −1 |
| ZNF563 | −1 |
| ZNF772 | −1 |
| ZSCAN18 | −1 |
| C2orf89 | −1 |
| PATL2 | +1 |
| TTC38 | +1 |
| PRR5L | +1 |
| SGK223 | −1 |
| NCRNA00287 | −1 |
| IGHM | −1 |
| HLA-DOA | −1 |
| IGHV5-78 | −1 |
| ABLIM1 | −1 |
| AFF3 | −1 |
| BACH2 | −1 |
| BCL11A | −1 |
| BIRC3 | −1 |
| BLNK | −1 |
| BTLA | −1 |
| C11orf31 | −1 |
| C6orf48 | −1 |
| CCR6 | −1 |
| CCR7 | −1 |
| CD200 | −1 |
| CD22 | −1 |
| CD27 | −1 |
| CD28 | −1 |
| CDCA7L | −1 |
| CHMP7 | −1 |
| CR2 | −1 |
| DPP4 | −1 |
| E2F5 | −1 |
| EPHX2 | −1 |
| FAIM3 | −1 |
| FAM102A | −1 |
| FAM134B | −1 |
| FCRL1 | −1 |
| FCRL2 | −1 |
| HOOK1 | −1 |
| HVCN1 | −1 |
| IL6ST | −1 |
| IMPDH2 | −1 |
| KIAA0748 | −1 |
| LEF1 | −1 |
| LRRN3 | −1 |
| MYC | −1 |
| NELL2 | −1 |
| NT5E | −1 |
| P2RX5 | −1 |
| PAQR8 | −1 |
| PLAG1 | −1 |
| PTPRK | −1 |
| RCAN3 | −1 |
| SCML1 | −1 |
| SLC7A6 | −1 |
| SNX9 | −1 |
| STRBP | −1 |
| SUSD3 | −1 |
| TCF4 | −1 |
| TCF7 | −1 |
| TCL1A | −1 |
| TCTN1 | −1 |
| UXT | −1 |
| VPREB3 | −1 |
| ZNF101 | −1 |
| ZNF671 | −1 |
| CRTC3 | −1 |
| STAP1 | −1 |
| HLA-DOB | −1 |

According to a particular embodiment, at least 20 genes are measured (and used to calculate immune age).

According to a particular embodiment at least 30 genes are measured used to calculate immune age).

According to a particular embodiment, at least 40 genes are measured (and used to calculate immune age).

According to a particular embodiment, at least 50 genes are measured (and used to calculate immune age).

According to a particular embodiment, at least 60 genes are measured (and used to calculate immune age).

According to a particular embodiment, no more than 60, 70, 80, 90, 100, 110 or 120 genes are, measured (and used to calculate immune age).

In one embodiment, at least the 5 following genes are measured: BACH2, BCL11A, CHMP7, DPP4 and LRRN3.

According to still another aspect of the present invention there is provided a method of analyzing the therapeutic efficacy of a therapeutic agent for treating an immune-related disorder of a subject:
(a) contacting blood cells of the subject with the agent;
(b) measuring the expression of each of the genes BACH2, BCL11A, CHMP7, DPP4 and LRRN3 in the blood cells of the subject;
(c) obtaining an immune age value based on the expression of the genes; and
(d) comparing the immune age value with an immune age value of the subject calculated on the basis of expression of the at genes in blood cells of the subject, in the absence of the agent, wherein a decrease in the immune age of the subject is indicative that the agent has therapeutic efficacy for treating the immune-related disorder of the subject.

Table 10 herein below summarizes details for each of the genes:

TABLE 10

| | | | | Length | | Length | |
|---|---|---|---|---|---|---|---|
| Gene ID | Symbol | UniProt ID | Transcript | (nt) | Protein | (aa) | Protein name |
| 11123 | RCAN3 | Q9UKA8 | NM_001251979.2 | 6668 | NP_001238908.1 | 241 | calcipressin-3 |
| 11123 | RCAN3 | Q9UKA8 | NM_001251984.2 | 6414 | NP_001238913.1 | 116 | calcipressin-3 |
| 11123 | RCAN3 | Q9UKA8 | NM_013441.4 | 6863 | NP_038469.1 | 241 | calcipressin-3 |
| 11123 | RCAN3 | Q9UKA8 | NM_001251983.2 | 6609 | NP_001238912.1 | 116 | calcipressin-3 |
| 11123 | RCAN3 | Q9UKA8 | NM_001251977.2 | 6830 | NP_001238906.1 | 241 | calcipressin-3 |
| 11123 | RCAN3 | Q9UKA8 | NM_001251981.2 | 6656 | NP_001238910.1 | 183 | calcipressin-3 |
| 11123 | RCAN3 | Q9UKA8 | NM_001251978.1 | 2668 | NP_001238907.1 | 241 | calcipressin-3 |
| 11123 | RCAN3 | Q9UKA8 | NM_001251980.1 | 2503 | NP_001238909.1 | 231 | calcipressin-3 |
| 11123 | RCAN3 | Q9UKA8 | NM_001251982.1 | 2361 | NP_001238911.1 | 173 | calcipressin-3 |
| 11123 | RCAN3 | Q9UKA8 | NM_001251985.1 | 2187 | NP_001238914.1 | 115 | calcipressin-3 |
| 114804 | RNF157 | Q96PX1 | XR_001752422.1 | 2288 | | | |
| 114804 | RNF157 | Q96PX1 | XM_005257007.4 | 4988 | XP_005257064.1 | 653 | E3 ubiquitin |
| | | | | | | | ligase RNF157 |
| 114804 | RNF157 | Q96PX1 | NM_001330501.2 | 4988 | NP_001317430.1 | 657 | E3 ubiquitin |
| | | | | | | | ligase RNF157 |
| 114804 | RNF157 | Q96PX1 | NM_052916.3 | 5054 | NP_443148.1 | 679 | E3 ubiquitin |
| | | | | | | | ligase RNF157 |
| 114804 | RNF157 | Q96PX1 | XM_017024117.2 | 5797 | XP_016879606.1 | 688 | E3 ubiquitin |
| | | | | | | | ligase RNF157 |
| 114804 | RNF157 | Q96PX1 | XM_017024120.2 | 5594 | XP_016879609.1 | 621 | E3 ubiquitin |
| | | | | | | | ligase RNF157 |
| 114804 | RNF157 | Q96PX1 | XM_011524273.3 | 1566 | XP_011522575.1 | 473 | E3 ubiquitin |
| | | | | | | | ligase RNF157 |
| 115350 | FCRL1 | Q96LA6 | XR_921738.1 | 5292 | | | |
| 115350 | FCRL1 | Q96LA6 | XR_921740.1 | 5300 | | | |
| 115350 | FCRL1 | Q96LA6 | XR_921739.1 | 1431 | | | |
| 115350 | FCRL1 | Q96LA6 | XM_005244869.3 | 4949 | XP_005244926.1 | 333 | Fc receptor- |
| | | | | | | | like protein 1 |
| 115350 | FCRL1 | Q96LA6 | XM_011509127.2 | 5216 | XP_011507429.1 | 422 | Fc receptor- |
| | | | | | | | like protein 1 |
| 115350 | FCRL1 | Q96LA6 | XM_011509135.2 | 4970 | XP_01150737.14 | 340 | Fc receptor- |
| | | | | | | | like protein 1 |
| 115350 | FCRL1 | Q96LA6 | XM_005244867.2 | 5107 | XP_005244924.1 | 390 | Fc receptor- |
| | | | | | | | like protein 1 |
| 115350 | FCRL1 | Q96LA6 | XM_01150130.19 | 5211 | XP_01150732.14 | 404 | Fc receptor- |
| | | | | | | | like protein 1 |
| 115350 | FCRL1 | Q96LA6 | XM_005244866.2 | 5214 | XP_005244923.1 | 405 | Fc receptor- |
| | | | | | | | like protein 1 |
| 115350 | FCRL1 | Q96LA6 | XM_011509129.1 | 5223 | XP_011507431.1 | 408 | Fc receptor- |
| | | | | | | | like protein 1 |
| 115350 | FCRL1 | Q96LA6 | XM_011509128.1 | 5226 | XP_011507430.1 | 409 | Fc receptor- |
| | | | | | | | like protein 1 |
| 115350 | FCRL1 | Q96LA6 | XM_011509126.1 | 5219 | XP_011507428.1 | 423 | Fc receptor- |
| | | | | | | | like protein 1 |
| 115350 | FCRL1 | Q96LA6 | XM_011509125.1 | 5222 | XP_011507427.1 | 424 | Fc receptor- |
| | | | | | | | like protein 1 |
| 115350 | FCRL1 | Q96LA6 | XM_011509133.2 | 5078 | XP_011507435.1 | 354 | Fc receptor- |
| | | | | | | | like protein 1 |
| 115350 | FCRL1 | Q96LA6 | XM_011509134.2 | 5053 | XP_011507436.1 | 354 | Fc receptor- |
| | | | | | | | like protein 1 |
| 115350 | FCRL1 | Q96LA6 | XM_017000227.1 | 7220 | XP_016855716.1 | 354 | Fc receptor- |
| | | | | | | | like protein 1 |
| 115350 | FCRL1 | Q96LA6 | XM_011509137.2 | 4598 | XP_011507439.1 | 219 | Fc receptor- |
| | | | | | | | like protein 1 |
| 115350 | FCRL1 | Q96LA6 | NM_001159397.2 | 2942 | NP_001152869.1 | 366 | Fc receptor- |
| | | | | | | | like protein 1 |
| 115350 | FCRL1 | Q96LA6 | NM_001159398.2 | 3066 | NP_001152870.1 | 428 | Fc receptor- |
| | | | | | | | like protein 1 |
| 115350 | FCRL1 | Q96LA6 | NM_052938.5 | 3069 | NP_443170.1 | 429 | Fc receptor- |
| | | | | | | | like protein 1 |
| 115350 | FCRL1 | Q96LA6 | XM_011509131.1 | 1373 | XP_011507433.1 | 395 | Fc receptor- |
| | | | | | | | like protein 1 |
| 115350 | FCRL1 | Q96LA6 | XM_011509132.2 | 1348 | XP_011507434.1 | 395 | Fc receptor- |
| | | | | | | | like protein 1 |
| 115350 | FCRL1 | Q96LA6 | XM_011509136.1 | 1078 | XP_011507438.1 | 299 | Fc receptor- |
| | | | | | | | like protein 1 |
| 1235 | CCR6 | P51684 | NM_004367.6 | 3228 | NP_004358.2 | 374 | |
| 1235 | CCR6 | P51684 | NM_001394582.1 | 3193 | NP_001381511.1 | 374 | |
| 1235 | CCR6 | P51684 | NM_031409.4 | 3136 | NP_113597.2 | 374 | |
| 1236 | CCR7 | P32248 | NM_001301714.2 | 2123 | NP_001288643.1 | 315 | C-C |
| | | | | | | | chemokine |
| | | | | | | | receptor type 7 |
| 1236 | CCR7 | P32248 | NM_001838.4 | 2173 | NP_001829.1 | 378 | C-C |
| | | | | | | | chemokine |
| | | | | | | | receptor type 7 |
| 1236 | CCR7 | P32248 | NM_001301717.2 | 2191 | NP_001288646.1 | 372 | C-C |
| | | | | | | | chemokine |
| | | | | | | | receptor type 7 |
| 1236 | CCR7 | P32248 | NM_001301716.2 | 2435 | NP_001288645.1 | 372 | C-C |
| | | | | | | | chemokine |
| | | | | | | | receptor type 7 |
| 1236 | CCR7 | P32248 | NM_001301718.2 | 2281 | NP_001288647.1 | 372 | C-C |
| | | | | | | | chemokine |
| | | | | | | | receptor type 7 |
| 129293 | TRABD2A | Q86V40 | XM_011532504.1 | 1791 | XP_011530806.1 | 402 | metalloprotease |
| | | | | | | | TIKI1 |
| 129293 | TRABD2A | Q86V40 | NM_001080824.3 | 1667 | NP_001074293.1 | 456 | metalloprotease |
| | | | | | | | TIKI1 |
| 129293 | TRABD2A | Q86V40 | NM_001277053.2 | 1814 | NP_001263982.1 | 505 | metalloprotease |
| | | | | | | | TIKI1 |
| 129293 | TRABD2A | Q86V40 | NM_001307978.2 | 3359 | NP_001294907.1 | 333 | metalloprotease |
| | | | | | | | TIKI1 |
| 1380 | CR2 | P20023 | NM_001006658.3 | 4139 | NP_001006659.1 | 1092 | complement |
| | | | | | | | receptor type 2 |
| 1380 | CR2 | P20023 | NM_001877.5 | 3962 | NP_001868.2 | 1033 | complement |
| | | | | | | | receptor type 2 |
| 1380 | CR2 | P20023 | XM_011509206.3 | 4056 | XP_011507508.1 | 969 | complement |
| | | | | | | | receptor type 2 |
| 147837 | ZNF563 | Q8TA94 | NM_001363608.1 | 2581 | NP_001350537.1 | 229 | zinc finger |
| | | | | | | | protein 563 |
| 147837 | ZNF563 | Q8TA94 | NM_145276.3 | 2699 | NP_660319.1 | 476 | zinc finger |
| | | | | | | | protein 563 |
| 147837 | ZNF563 | Q8TA94 | XM_011527700.2 | 1537 | XP_011526002.1 | 439 | zinc finger |
| | | | | | | | protein 563 |
| 147837 | ZNF563 | Q8TA94 | XM_005259750.4 | 1239 | XP_005259807.1 | 302 | zinc finger |
| | | | | | | | protein 563 |
| 147837 | ZNF563 | Q8TA94 | XM_006722650.3 | 1699 | XP_006722713.1 | 439 | zinc finger |
| | | | | | | | protein 563 |
| 147837 | ZNF563 | Q8TA94 | XM_006722651.3 | 1592 | XP_006722714.1 | 420 | zinc finger |
| | | | | | | | protein 563 |
| 147837 | ZNF563 | Q8TA94 | XM_01152699.27 | 1766 | XP_011526001.1 | 439 | zinc finger |
| | | | | | | | protein 563 |
| 147837 | ZNF563 | Q8TA94 | XM_011527698.2 | 1822 | XP_011526000.1 | 444 | zinc finger |
| | | | | | | | protein 563 |
| 151888 | BTLA | Q7Z6A9 | XM_011512447.3 | 4264 | XP_011510749.1 | 295 | B- and T- |
| | | | | | | | lymphocyte |
| | | | | | | | attenuator |
| 151888 | BTLA | Q7Z6A9 | XM_017005748.2 | 3294 | XP_016861237.1 | 289 | B- and T- |
| | | | | | | | lymphocyte |
| | | | | | | | attenuator |
| 151888 | BTLA | Q7Z6A9 | NM_001085357.2 | 2982 | NP_001078826.1 | 241 | B- and T- |
| | | | | | | | lymphocyte |
| | | | | | | | attenuator |
| 151888 | BTLA | Q7Z6A9 | NM_181780.4 | 3126 | NP_861445.4 | 289 | B- and T- |
| | | | | | | | lymphocyte |
| | | | | | | | attenuator |
| 1521 | CTSW | P56202 | NM_001335.4 | 1272 | NP_001326.3 | 376 | |
| 157285 | PRAG1 | Q86YV5 | NR_163138.1 | 4936 | | | |
| 157285 | PRAG1 | Q86YV5 | NM_001080826.3 | 4845 | NP_001074295.2 | 1406 | |
| 157285 | PRAG1 | Q86YV5 | NM_001369759.1 | 5316 | NP_001356688.1 | 1406 | |
| 1803 | DPP4 | P27487 | NR_166822.1 | 3805 | | | |
| 1803 | DPP4 | P27487 | NR_166825.1 | 3520 | | | |
| 1803 | DPP4 | P27487 | NR_166824.1 | 3571 | | | |
| 1803 | DPP4 | P27487 | NR_166823.1 | 3813 | | | |
| 1803 | DPP4 | P27487 | NM_001379605.1 | 3567 | NP_001366534.1 | 764 | dipeptidyl |
| | | | | | | | peptidase 4 |
| 1803 | DPP4 | P27487 | NM_001379604.1 | 3570 | NP_001366533.1 | 765 | dipeptidyl |
| | | | | | | | peptidase 4 |
| 1803 | DPP4 | P27487 | NM_001379606.1 | 3519 | NP_001366535.1 | 748 | dipeptidyl |
| | | | | | | | peptidase 4 |
| 1803 | DPP4 | P27487 | NM_001935.4 | 3573 | NP_001926.2 | 766 | dipeptidyl |
| | | | | | | | peptidase 4 |
| 1875 | E2F5 | Q15329 | NM_001951.4 | 1964 | NP_001942.2 | 346 | transcription |
| | | | | | | | factor E2F5 |
| 1875 | E2F5 | Q15329 | NM_001083588.2 | 1961 | NP_001077057.1 | 345 | transcription |
| | | | | | | | factor E2F5 |
| 1875 | E2F5 | Q15329 | NM_001083589.2 | 1595 | NP_001077058.1 | 185 | transcription |
| | | | | | | | factor E2F5 |
| 1933 | EEF1B2 | P24534 | NM_001037663.2 | 866 | NP_001032752.1 | 225 | |
| 1933 | EEF1B2 | P24534 | NM_021121.4 | 820 | NP_066944.1 | 225 | |
| 1933 | EEF1B2 | P24534 | NM_001959.4 | 808 | NP_001950.1 | 225 | |
| 197135 | PATL2 | C9JE40 | XM_011521340.3 | 2217 | XP_011519642.1 | 565 | protein PAT1 |
| | | | | | | | homolog 2 |
| 197135 | PATL2 | C9JE40 | XM_011521339.3 | 2276 | XP_011519641.1 | 565 | protein PAT1 |
| | | | | | | | homolog 2 |
| 197135 | PATL2 | C9JE40 | XM_011521345.3 | 2189 | XP_011519647.1 | 478 | protein PAT1 |
| | | | | | | | homolog 2 |
| 197135 | PATL2 | C9JE40 | XM_011521343.3 | 2227 | XP_011519645.1 | 481 | protein PAT1 |
| | | | | | | | homolog 2 |
| 197135 | PATL2 | C9JE40 | XM_011521338.3 | 2395 | XP_011519640.1 | 565 | protein PAT1 |
| | | | | | | | homolog 2 |
| 197135 | PATL2 | C9JE40 | NM_001387260.1 | 1894 | NP_001374189.1 | 512 | protein PAT1 |
| | | | | | | | homolog 2 |
| 197135 | PATL2 | C9JE40 | NM_001387261.1 | 2232 | NP_001374190.1 | 543 | protein PAT1 |
| | | | | | | | homolog 2 |
| 197135 | PATL2 | C9JE40 | NM_001387263.1 | 2410 | NP_001374192.1 | 543 | protein PAT1 |
| | | | | | | | homolog 2 |
| 197135 | PATL2 | C9JE40 | NM_001387262.1 | 2500 | NP_001374191.1 | 543 | protein PAT1 |
| | | | | | | | homolog 2 |
| 197135 | PATL2 | C9JE40 | XM_011521337.2 | 2814 | XP_011519639.2 | 600 | protein PAT1 |
| | | | | | | | homolog 2 |
| 197135 | PATL2 | C9JE40 | XM_011521346.2 | 2386 | XP_011519648.2 | 458 | protein PAT1 |
| | | | | | | | homolog 2 |
| 197135 | PATL2 | C9JE40 | XM_011521336.2 | 2679 | XP_011519638.2 | 603 | protein PAT1 |
| | | | | | | | homolog 2 |
| 197135 | PATL2 | C9JE40 | XM_011521347.1 | 1768 | XP_011519649.1 | 376 | protein PAT1 |
| | | | | | | | homolog 2 |
| 197135 | PATL2 | C9JE40 | NM_001330283.2 | 1852 | NP_001317212.1 | 354 | protein PAT1 |
| | | | | | | | homolog 2 |
| 197135 | PATL2 | C9JE40 | NM_001145112.2 | 1937 | NP_001138584.1 | 543 | protein PAT1 |
| | | | | | | | homolog 2 |
| 197135 | PATL2 | C9JE40 | NM_001387264.1 | 1983 | NP_001374193.1 | 512 | protein PAT1 |
| | | | | | | | homolog 2 |
| 197135 | PATL2 | C9JE40 | XM_011521341.1 | 1963 | XP_011519643.1 | 565 | protein PAT1 |
| | | | | | | | homolog 2 |
| 197135 | PATL2 | C9JE40 | XM_011521342.2 | 2860 | XP_011519644.1 | 491 | protein PAT1 |
| | | | | | | | homolog 2 |
| 197135 | PATL2 | C9JE40 | XM_011521344.3 | 1650 | XP_011519646.1 | 481 | protein PAT1 |
| | | | | | | | homolog 2 |
| 197135 | PATL2 | C9JE40 | XM_017022001.2 | 1624 | XP_016877490.1 | 478 | protein PAT1 |
| | | | | | | | homolog 2 |
| 199786 | NIBAN3 | Q86XR2 | NM_173544.5 | 2512 | NP_775815.3 | 697 | protein Niban |
| | | | | | | | 3 |
| 199786 | NIBAN3 | Q86XR2 | NM_001098524.2 | 3836 | NP_001091994.2 | 651 | protein Niban |
| | | | | | | | 3 |
| 199786 | NIBAN3 | Q86XR2 | XM_017026455.1 | 2322 | XP_016881944.1 | 495 | protein Niban |
| | | | | | | | 3 |
| 199786 | NIBAN3 | Q86XR2 | XM_011527786.2 | 3450 | XP_011526088.1 | 591 | protein Niban |
| | | | | | | | 3 |
| 199786 | NIBAN3 | Q86XR2 | XM_017026453.1 | 2450 | XP_016881942.1 | 738 | protein Niban |
| | | | | | | | 3 |
| 199786 | NIBAN3 | Q86XR2 | XM_017026454.1 | 2342 | XP_016881943.1 | 702 | protein Niban |
| | | | | | | | 3 |
| 199786 | NIBAN3 | Q86XR2 | XM_011527787.3 | 3492 | XP_011526089.1 | 586 | protein Niban |
| | | | | | | | 3 |
| 199786 | NIBAN3 | Q86XR2 | XM_011527781.3 | 2170 | XP_011526083.1 | 683 | protein Niban |
| | | | | | | | 3 |
| 199786 | NIBAN3 | Q86XR2 | XM_005259813.4 | 1948 | XP_005259870.1 | 592 | protein Niban |
| | | | | | | | 3 |
| 199786 | NIBAN3 | Q86XR2 | XM_011527789.1 | 1719 | XP_011526091.1 | 519 | protein Niban |
| | | | | | | | 3 |
| 199786 | NIBAN3 | Q86XR2 | NM_001321826.2 | 2355 | NP_001308755.2 | 666 | protein Niban |
| | | | | | | | 3 |
| 199786 | NIBAN3 | Q86XR2 | XM_017026456.1 | 2377 | XP_016881945.1 | 495 | protein Niban |
| | | | | | | | 3 |
| 199786 | NIBAN3 | Q86XR2 | NM_001321827.2 | 3679 | NP_001308756.2 | 620 | protein Niban |
| | | | | | | | 3 |
| 199786 | NIBAN3 | Q86XR2 | NM_001363609.1 | 1957 | NP_001350538.1 | 584 | protein Niban |
| | | | | | | | 3 |
| 199786 | NIBAN3 | Q86XR2 | NM_001321828.1 | 2233 | NP_001308757.1 | 423 | protein Niban |
| | | | | | | | 3 |
| 199786 | NIBAN3 | Q86XR2 | XM_017026457.1 | 2027 | XP_016881946.1 | 495 | protein Niban |
| | | | | | | | 3 |
| 203328 | SUSD3 | Q96L08 | NM_001287005.2 | 1310 | NP_001273934.1 | 242 | sushi domain- |
| | | | | | | | containing |
| | | | | | | | protein 3 |
| 203328 | SUSD3 | Q96L08 | NM_145006.4 | 1196 | NP_659443.1 | 255 | sushi domain- |
| | | | | | | | containing |
| | | | | | | | protein 3 |
| 203328 | SUSD3 | Q96L08 | NM_001287006.2 | 1064 | NP_001273935.1 | 211 | sushi domain- |
| | | | | | | | containing |
| | | | | | | | protein 3 |
| 203328 | SUSD3 | Q96L08 | NM_001287008.2 | 1007 | NP_001273937.1 | 192 | sushi domain- |
| | | | | | | | containing |
| | | | | | | | protein 3 |
| 203328 | SUSD3 | Q96L08 | NM_001287007.2 | 875 | NP_001273936.1 | 148 | sushi domain- |
| | | | | | | | containing |
| | | | | | | | protein 3 |
| 203328 | SUSD3 | Q96L08 | XM_017014450.1 | 1355 | XP_016869939.1 | 242 | sushi domain- |
| | | | | | | | containing |
| | | | | | | | protein 3 |
| 203328 | SUSD3 | Q96L08 | XM_017014448.1 | 1515 | XP_016869937.1 | 271 | sushi domain- |
| | | | | | | | containing |
| | | | | | | | protein 3 |
| 203328 | SUSD3 | Q96L08 | XM_017014449.1 | 1396 | XP_016869938.1 | 271 | sushi domain- |
| | | | | | | | containing |
| | | | | | | | protein 3 |
| 203328 | SUSD3 | Q96L08 | XM_011518361.2 | 1470 | XP_011516663.1 | 242 | sushi domain- |
| | | | | | | | containing |
| | | | | | | | protein 3 |
| 203328 | SUSD3 | Q96L08 | XM_011518358.1 | 2408 | XP_011516660.1 | 242 | sushi domain- |
| | | | | | | | containing |
| | | | | | | | protein 3 |
| 203328 | SUSD3 | Q96L08 | XM_011518359.1 | 2358 | XP_011516661.1 | 242 | sushi domain- |
| | | | | | | | containing |
| | | | | | | | protein 3 |
| 203328 | SUSD3 | Q96L08 | XM_011518362.1 | 2244 | XP_011516664.1 | 198 | sushi domain- |
| | | | | | | | containing |
| | | | | | | | protein 3 |
| 203328 | SUSD3 | Q96L08 | XM_017014451.2 | 1723 | XP_016869940.1 | 233 | sushi domain- |
| | | | | | | | containing |
| | | | | | | | protein 3 |
| 2053 | EPHX2 | P34913 | XR_001745491.1 | 2420 | | | |
| 2053 | EPHX2 | P34913 | NM_001979.6 | 2776 | NP_001970.2 | 555 | bifunctional |
| | | | | | | | epoxide |
| | | | | | | | hydrolase 2 |
| 2053 | EPHX2 | P34913 | NM_001256484.2 | 2774 | NP_001243413.1 | 502 | bifunctional |
| | | | | | | | epoxide |
| | | | | | | | hydrolase 2 |
| 2053 | EPHX2 | P34913 | NM_001256482.2 | 2738 | NP_001243411.1 | 502 | bifunctional |
| | | | | | | | epoxide |
| | | | | | | | hydrolase 2 |
| 2053 | EPHX2 | P34913 | NM_001256483.2 | 2691 | NP_001243412.1 | 489 | bifunctional |
| | | | | | | | epoxide |
| | | | | | | | hydrolase 2 |
| 2053 | EPHX2 | P34913 | XM_017013199.1 | 2001 | XP_016868688.1 | 514 | bifunctional |
| | | | | | | | epoxide |
| | | | | | | | hydrolase 2 |
| 2053 | EPHX2 | P34913 | XM_017013200.1 | 2192 | XP_016868689.1 | 300 | bifunctional |
| | | | | | | | epoxide |
| | | | | | | | hydrolase 2 |
| 2208 | FCER2 | P06734 | XM_005272462.4 | 2218 | XP_005272519.1 | 321 | low affinity |
| | | | | | | | immunoglobulin |
| | | | | | | | epsilon Fc |
| | | | | | | | receptor |
| 2208 | FCER2 | P06734 | NM_002002.5 | 1581 | NP_001993.2 | 321 | low affinity |
| | | | | | | | immunoglobulin |
| | | | | | | | epsilon Fc |
| | | | | | | | receptor |
| 2208 | FCER2 | P06734 | NM_001220500.2 | 1585 | NP_001207429.1 | 321 | low affinity |
| | | | | | | | immunoglobulin |
| | | | | | | | epsilon Fc |
| | | | | | | | receptor |
| 2208 | FCER2 | P06734 | NM_001207019.3 | 1426 | NP_001193948.2 | 320 | low affinity |
| | | | | | | | immunoglobulin |
| | | | | | | | epsilon Fc |
| | | | | | | | receptor |
| 2323 | FLT3LG | P49771 | XR_935782.2 | 1098 | | | |
| 2323 | FLT3LG | P49771 | XR_935781.2 | 1067 | | | |
| 2323 | FLT3LG | P49771 | XM_017026533.1 | 803 | XP_016882022.1 | 208 | fms-related |
| | | | | | | | tyrosine kinase |
| | | | | | | | 3 ligand |
| 2323 | FLT3LG | P49771 | XM_011526675.2 | 913 | XP_011524977.1 | 245 | fms-related |
| | | | | | | | tyrosine kinase |
| | | | | | | | 3 ligand |
| 2323 | FLT3LG | P49771 | XM_011526678.2 | 859 | XP_011524980.1 | 227 | fms-related |
| | | | | | | | tyrosine kinase |
| | | | | | | | 3 ligand |
| 2323 | FLT3LG | P49771 | XM_017026535.1 | 1025 | XP_016882024.1 | 135 | fms-related |
| | | | | | | | tyrosine kinase |
| | | | | | | | 3 ligand |
| 2323 | FLT3LG | P49771 | NM_001278638.2 | 1067 | NP_001265567.1 | 153 | fms-related |
| | | | | | | | tyrosine kinase |
| | | | | | | | 3 ligand |
| 2323 | FLT3LG | P49771 | NM_001278637.2 | 1078 | NP_001265566.1 | 153 | fms-related |
| | | | | | | | tyrosine kinase |
| | | | | | | | 3 ligand |
| 2323 | FLT3LG | P49771 | NM_001204502.2 | 1101 | NP_001191431.1 | 235 | fms-related |
| | | | | | | | tyrosine kinase |
| | | | | | | | 3 ligand |
| 2323 | FLT3LG | P49771 | NM_001459.4 | 1050 | NP_001450.2 | 235 | fms-related |
| | | | | | | | tyrosine kinase |
| | | | | | | | 3 ligand |
| 2323 | FLT3LG | P49771 | XM_017026534.2 | 777 | XP_016882023.1 | 208 | fms-related |
| | | | | | | | tyrosine kinase |
| | | | | | | | 3 ligand |
| 2323 | FLT3LG | P49771 | XM_011526676.3 | 879 | XP_011524978.1 | 245 | fms-related |
| | | | | | | | tyrosine kinase |
| | | | | | | | 3 ligand |
| 2323 | FLT3LG | P49771 | XM_005258682.5 | 1765 | XP_005258739.3 | 217 | fms-related |
| | | | | | | | tyrosine kinase |
| | | | | | | | 3 ligand |
| 2323 | FLT3LG | P49771 | XM_017026532.1 | 734 | XP_016882021.1 | 196 | fms-related |
| | | | | | | | tyrosine kinase |
| | | | | | | | 3 ligand |
| 2323 | FLT3LG | P49771 | XM_006723116.3 | 730 | XP_006723179.2 | 196 | fms-related |
| | | | | | | | tyrosine kinase |
| | | | | | | | 3 ligand |
| 2323 | FLT3LG | P49771 | XM_011526682.2 | 1063 | XP_011524984.1 | 153 | fms-related |
| | | | | | | | tyrosine kinase |
| | | | | | | | 3 ligand |
| 2323 | FLT3LG | P49771 | XM_005258681.4 | 1043 | XP_005258738.3 | 217 | fms-related |
| | | | | | | | tyrosine kinase |
| | | | | | | | 3 ligand |
| 2323 | FLT3LG | P49771 | XM_005258680.4 | 992 | XP_005258737.3 | 217 | fms-related |
| | | | | | | | tyrosine kinase |
| | | | | | | | 3 ligand |
| 2323 | FLT3LG | P49771 | XM_005258683.4 | 907 | XP_005258740.3 | 200 | fms-related |
| | | | | | | | tyrosine kinase |
| | | | | | | | 3 ligand |
| 2323 | FLT3LG | P49771 | XM_011526680.2 | 525 | XP_011524982.1 | 130 | fms-related |
| | | | | | | | tyrosine kinase |
| | | | | | | | 3 ligand |
| 2323 | FLT3LG | P49771 | XM_011526677.2 | 744 | XP_011524979.1 | 186 | fms-related |
| | | | | | | | tyrosine kinase |
| | | | | | | | 3 ligand |
| 2323 | FLT3LG | P49771 | NM_001204503.2 | 1051 | NP_001191432.1 | 235 | fms-related |
| | | | | | | | tyrosine kinase |
| | | | | | | | 3 ligand |
| 23331 | TTC28 | Q96AY4 | NM_001393403.1 | 11746 | NP_001380332.1 | 2473 | tetratricopeptide |
| | | | | | | | repeat protein |
| | | | | | | | 28 |
| 23331 | TTC28 | Q96AY4 | NM_001145418.2 | 11770 | NP_001138890.1 | 2481 | tetratricopeptide |
| | | | | | | | repeat protein |
| | | | | | | | 28 |
| 23331 | TTC28 | Q96AY4 | XM_011530018.3 | 11975 | XP_011528320.1 | 2455 | tetratricopeptide |
| | | | | | | | repeat protein |
| | | | | | | | 28 |
| 23331 | TTC28 | Q96AY4 | XM_017028673.2 | 11769 | XP_016884162.1 | 2451 | tetratricopeptide |
| | | | | | | | repeat protein |
| | | | | | | | 28 |
| 23331 | TTC28 | Q96AY4 | NM_001393405.1 | 12022 | NP_001380334.1 | 2355 | tetratricopeptide |
| | | | | | | | repeat protein |
| | | | | | | | 28 |
| 23331 | TTC28 | Q96AY4 | NM_001393404.1 | 12046 | NP_001380333.1 | 2363 | tetratricopeptide |
| | | | | | | | repeat protein |
| | | | | | | | 28 |
| 23331 | TTC28 | Q96AY4 | XM_011530019.2 | 6558 | XP_011528321.1 | 1412 | tetratricopeptide |
| | | | | | | | repeat protein |
| | | | | | | | 28 |
| 23331 | TTC28 | Q96AY4 | XM_011530021.3 | 6615 | XP_011528323.1 | 1390 | tetratricopeptide |
| | | | | | | | repeat protein |
| | | | | | | | 28 |
| 23331 | TTC28 | Q96AY4 | XM_011530022.1 | 4984 | XP_011528324.1 | 1390 | tetratricopeptide |
| | | | | | | | repeat protein |
| | | | | | | | 28 |
| 23331 | TTC28 | Q96AY4 | XM_011530020.1 | 4508 | XP_011528322.1 | 1400 | tetratricopeptide |
| | | | | | | | repeat protein |
| | | | | | | | 28 |
| 26228 | STAP1 | Q9ULZ2 | NM_012108.4 | 1951 | NP_036240.1 | 295 | |
| 26228 | STAP1 | Q9ULZ2 | NM_001317769.2 | 1147 | NP_001304698.1 | 295 | |
| 26228 | STAP1 | Q9ULZ2 | XM_017008018.2 | 1220 | XP_016863507.1 | 314 | signal- |
| | | | | | | | transducing |
| | | | | | | | adaptor |
| | | | | | | | protein 1 |
| 27075 | TSPAN13 | O95857 | NM_014399.4 | 1873 | NP_055214.1 | 204 | |
| 280636 | SELENOH | Q8IZQ | NM_170746.4 | 1192 | NP_734467.1 | 122 | |
| 280636 | SELENOH | Q8IZQ | NM_001321335.2 | 1172 | NP_001308264.1 | 122 | |
| 28387 | IGHV5-78 | | | | | | |
| 29760 | BLNK | Q8WV28 | NR_047680.1 | 4168 | | | |
| 29760 | BLNK | Q8WV28 | NR_047682.2 | 4111 | | | |
| 29760 | BLNK | Q8WV28 | NR_047683.2 | 4049 | | | |
| 29760 | BLNK | Q8WV28 | NR_047681.2 | 4116 | | | |
| 29760 | BLNK | Q8WV28 | NM_001258442.2 | 3873 | NP_001245371.1 | 299 | B-cell linker |
| | | | | | | | protein |
| 29760 | BLNK | Q8WV28 | NM_001258441.2 | 4119 | NP_001245370.1 | 381 | B-cell linker |
| | | | | | | | protein |
| 29760 | BLNK | Q8WV28 | NM_001114094.2 | 4275 | NP_001107566.1 | 433 | B-cell linker |
| | | | | | | | protein |
| 29760 | BLNK | Q8WV28 | NM_001258440.2 | 4188 | NP_001245369.1 | 404 | B-cell linker |
| | | | | | | | protein |
| 29760 | BLNK | Q8WV28 | NM_013314.4 | 4344 | NP_037446.1 | 456 | B-cell linker |
| | | | | | | | protein |
| 29799 | YPEL1 | O60688 | NR_130910.2 | 4681 | | | |
| 29799 | YPEL1 | O60688 | NM_013313.5 | 4297 | NP_037445.1 | 119 | |
| 29802 | VPREB3 | Q9UKI3 | NM_013378.3 | 567 | NP_037510.1 | 123 | |
| 2999 | GZMH | P20718 | XM_011536683.2 | 1027 | XP_011534985.1 | 212 | granzyme H |
| 2999 | GZMH | P20718 | NM_001270781.2 | 678 | NP_001257710.1 | 160 | granzyme H |
| 2999 | GZMH | P20718 | NM_001270780.2 | 744 | NP_001257709.1 | 182 | granzyme H |
| 2999 | GZMH | P20718 | NM_033423.5 | 936 | NP_219491.1 | 246 | granzyme H |
| 30009 | TBX21 | Q9UL17 | NM_013351.2 | 2583 | NP_037483.1 | 535 | |
| 3111 | HLA-DOA | P06340 | NM_002119.4 | 3464 | NP_002110.1 | 250 | |
| 3112 | HLA-DOB | P13765 | NM_002120.4 | 1326 | NP_002111.1 | 273 | |
| 322 | APBB1 | O00213 | NR_047512.2 | 2277 | | | |
| 322 | APBB1 | O00213 | NM_001257320.2 | 2114 | NP_001244249.1 | 45 | amyloid-beta |
| | | | | | | | A4 precursor |
| | | | | | | | protein- |
| | | | | | | | binding family |
| | | | | | | | B member 1 |
| 322 | APBB1 | O00213 | NM_001257326.2 | 2122 | NP_001244255.1 | 451 | amyloid-beta |
| | | | | | | | A4 precursor |
| | | | | | | | protein- |
| | | | | | | | binding family |
| | | | | | | | B member 1 |
| 322 | APBB1 | O00213 | NM_001257321.2 | 2162 | NP_001244250.1 | 45 | amyloid-beta |
| | | | | | | | A4 precursor |
| | | | | | | | protein- |
| | | | | | | | binding family |
| | | | | | | | B member 1 |
| 322 | APBB1 | O00213 | NM_145689.3 | 2658 | NP_663722.1 | 708 | amyloid-beta |
| | | | | | | | A4 precursor |
| | | | | | | | protein- |
| | | | | | | | binding family |
| | | | | | | | B member 1 |
| 322 | APBB1 | O00213 | NM_001164.5 | 2666 | NP_001155.1 | 710 | amyloid-beta |
| | | | | | | | A4 precursor |
| | | | | | | | protein- |
| | | | | | | | binding family |
| | | | | | | | B member 1 |
| 322 | APBB1 | O00213 | NM_001257323.3 | 2004 | NP_001244252.1 | 488 | amyloid-beta |
| | | | | | | | A4 precursor |
| | | | | | | | protein- |
| | | | | | | | binding family |
| | | | | | | | B member 1 |
| 322 | APBB1 | O00213 | NM_001257319.3 | 2010 | NP_001244248.1 | 490 | amyloid-beta |
| | | | | | | | A4 precursor |
| | | | | | | | protein- |
| | | | | | | | binding family |
| | | | | | | | B member 1 |
| 322 | APBB1 | O00213 | NM_001257325.3 | 1906 | NP_001244254.1 | 475 | amyloid-beta |
| | | | | | | | A4 precursor |
| | | | | | | | protein- |
| | | | | | | | binding family |
| | | | | | | | B member 1 |
| 3221 | HOXC4 | P09017 | NM_014620.6 | 2270 | NP_055435.2 | 264 | |
| 3221 | HOXC4 | P09017 | NM_153633.3 | 1708 | NP_705897.1 | 264 | |
| 330 | BIRC3 | Q13489 | XM_024448467.1 | 3381 | XP_024304235.1 | 381 | baculoviral |
| | | | | | | | IAP repeat- |
| | | | | | | | containing |
| | | | | | | | protein 3 |
| 330 | BIRC3 | Q13489 | NM_001165.5 | 6877 | NP_001156.1 | 604 | |
| 330 | BIRC3 | Q13489 | NM_182962.3 | 4317 | NP_892007.1 | 604 | |
| 3399 | ID3 | Q02535 | NM_002167.5 | 950 | NP_002158.3 | 119 | |
| 343413 | FCRL6 | Q6DN72 | XM_006711292.3 | 2014 | XP_006711355.1 | 416 | Fc receptor- |
| | | | | | | | like protein 6 |
| 343413 | FCRL6 | Q6DN72 | XM_005245131.3 | 1987 | XP_005245188.1 | 407 | Fc receptor- |
| | | | | | | | like protein 6 |
| 343413 | FCRL6 | Q6DN72 | XM_011509481.2 | 1282 | XP_011507783.1 | 371 | Fc receptor- |
| | | | | | | | like protein 6 |
| 343413 | FCRL6 | Q6DN72 | NM_001284217.2 | 2003 | NP_001271146.1 | 413 | Fc receptor- |
| | | | | | | | like protein 6 |
| 343413 | FCRL6 | Q6DN72 | XM_011509480.2 | 2002 | XP_011507782.1 | 441 | Fc receptor- |
| | | | | | | | like protein 6 |
| 343413 | FCRL6 | Q6DN72 | XM_017001176.1 | 1872 | XP_016856665.1 | 397 | Fc receptor- |
| | | | | | | | like protein 6 |
| 343413 | FCRL6 | Q6DN72 | XM_005245129.4 | 1962 | XP_005245186.1 | 429 | Fc receptor- |
| | | | | | | | like protein 6 |
| 343413 | FCRL6 | Q6DN72 | XM_005245128.4 | 1995 | XP_005245185.1 | 444 | Fc receptor- |
| | | | | | | | like protein 6 |
| 343413 | FCRL6 | Q6DN72 | XM_017001177.1 | 1805 | XP_016856666.1 | 365 | Fc receptor- |
| | | | | | | | like protein 6 |
| 343413 | FCRL6 | Q6DN72 | NM_001004310.3 | 1966 | NP_001004310.2 | 434 | Fc receptor- |
| | | | | | | | like protein 6 |
| 3507 | IGHM | P01871 | | | | | |
| 3572 | IL6ST | P40189 | NR_157112.2 | 9908 | | | |
| 3572 | IL6ST | P40189 | NR_120480.2 | 9073 | | | |
| 3572 | IL6ST | P40189 | NM_001364276.2 | 8817 | NP_001351205.1 | 848 | interleukin-6 |
| | | | | | | | receptor |
| | | | | | | | subunit beta |
| 3572 | IL6ST | P40189 | NM_001364279.2 | 8823 | NP_001351208.1 | 586 | interleukin-6 |
| | | | | | | | receptor |
| | | | | | | | subunit beta |
| 3572 | IL6ST | P40189 | NM_001364278.2 | 8906 | NP_001351207.1 | 617 | interleukin-6 |
| | | | | | | | receptor |
| | | | | | | | subunit beta |
| 3572 | IL6ST | P40189 | NM_001364277.2 | 8952 | NP_001351206.1 | 629 | interleukin-6 |
| | | | | | | | receptor |
| | | | | | | | subunit beta |
| 3572 | IL6ST | P40189 | NM_175767.3 | 8944 | NP_786943.1 | 329 | interleukin-6 |
| | | | | | | | receptor |
| | | | | | | | subunit beta |
| 3572 | IL6ST | P40189 | NM_001190981.2 | 8844 | NP_001177910.1 | 857 | interleukin-6 |
| | | | | | | | receptor |
| | | | | | | | subunit beta |
| 3572 | IL6ST | P40189 | NM_001364275.2 | 8925 | NP_001351204.1 | 884 | interleukin-6 |
| | | | | | | | receptor |
| | | | | | | | subunit beta |
| 3572 | IL6ST | P40189 | NM_002184.4 | 9027 | NP_002175.2 | 918 | interleukin-6 |
| | | | | | | | receptor |
| | | | | | | | subunit beta |
| 3615 | IMPDH2 | P12268 | XM_006713128.3 | 2075 | XP_006713191.1 | 608 | inosine-5′- |
| | | | | | | | monophosphate |
| | | | | | | | dehydrogenase |
| | | | | | | | 2 |
| 3615 | IMPDH2 | P12268 | XM_017006350.1 | 1880 | XP_016861839.1 | 559 | inosine-5′- |
| | | | | | | | monophosphate |
| | | | | | | | dehydrogenase |
| | | | | | | | 2 |
| 3615 | IMPDH2 | P12268 | XM_017006349.1 | 1952 | XP_016861838.1 | 583 | inosine-5′- |
| | | | | | | | monophosphate |
| | | | | | | | dehydrogenase |
| | | | | | | | 2 |
| 3615 | IMPDH2 | P12268 | NM_000884.3 | 1651 | NP_000875.2 | 514 | |
| 3899 | AFF3 | P51826 | NM_002285.3 | 9987 | NP_002276.2 | 1226 | AF4/FMR2 |
| | | | | | | | family member |
| | | | | | | | 3 |
| 3899 | AFF3 | P51826 | NM_001386135.1 | 10070 | NP_001373064.1 | 1226 | AF4/FMR2 |
| | | | | | | | family member |
| | | | | | | | 3 |
| 3899 | AFF3 | P51826 | NM_001025108.2 | 9955 | NP_001020279.1 | 1251 | AF4/FMR2 |
| | | | | | | | family member |
| | | | | | | | 3 |
| 3899 | AFF3 | P51826 | XM_011511171.3 | 11208 | XP_011509473.2 | 1364 | AF4/FMR2 |
| | | | | | | | family member |
| | | | | | | | 3 |
| 3899 | AFF3 | P51826 | XM_024452883.1 | 11256 | XP_024308651.1 | 1380 | AF4/FMR2 |
| | | | | | | | family member |
| | | | | | | | 3 |
| 3899 | AFF3 | P51826 | XM_011511170.2 | 8257 | XP_011509472.1 | 1303 | AF4/FMR2 |
| | | | | | | | family member |
| | | | | | | | 3 |
| 3899 | AFF3 | P51826 | XM_011511177.3 | 7688 | XP_011509479.1 | 1226 | AF4/FMR2 |
| | | | | | | | family member |
| | | | | | | | 3 |
| 3899 | AFF3 | P51826 | XM_011511175.3 | 7907 | XP_011509477.1 | 1226 | AF4/FMR2 |
| | | | | | | | family member |
| | | | | | | | 3 |
| 3899 | AFF3 | P51826 | XM_011511169.3 | 9400 | XP_011509471.2 | 1379 | AF4/FMR2 |
| | | | | | | | family member |
| | | | | | | | 3 |
| 3899 | AFF3 | P51826 | XM_011511176.3 | 7598 | XP_011509478.1 | 1226 | AF4/FMR2 |
| | | | | | | | family member |
| | | | | | | | 3 |
| 3899 | AFF3 | P51826 | XM_011511173.3 | 7673 | XP_011509475.1 | 1251 | AF4/FMR2 |
| | | | | | | | family member |
| | | | | | | | 3 |
| 3899 | AFF3 | P51826 | XM_005263943.4 | 7554 | XP_005264000.2 | 1226 | AF4/FMR2 |
| | | | | | | | family member |
| | | | | | | | 3 |
| 3899 | AFF3 | P51826 | XM_011511174.3 | 7617 | XP_011509476.1 | 1251 | AF4/FMR2 |
| | | | | | | | family member |
| | | | | | | | 3 |
| 3899 | AFF3 | P51826 | XM_017004085.2 | 12103 | XP_016859574.1 | 894 | AF4/FMR2 |
| | | | | | | | family member |
| | | | | | | | 3 |
| 3899 | AFF3 | P51826 | XM_005263945.4 | 12106 | XP_005264002.1 | 895 | AF4/FMR2 |
| | | | | | | | family member |
| | | | | | | | 3 |
| 3899 | AFF3 | P51826 | XM_017004087.2 | 8564 | XP_016859576.1 | 861 | AF4/FMR2 |
| | | | | | | | family member |
| | | | | | | | 3 |
| 3899 | AFF3 | P51826 | XM_017004086.2 | 8567 | XP_016859575.1 | 862 | AF4/FMR2 |
| | | | | | | | family member |
| | | | | | | | 3 |
| 3899 | AFF3 | P51826 | XM_011511179.3 | 6416 | XP_011509481.1 | 864 | AF4/FMR2 |
| | | | | | | | family member |
| | | | | | | | 3 |
| 3899 | AFF3 | P51826 | XM_011511178.3 | 6419 | XP_011509480.1 | 865 | AF4/FMR2 |
| | | | | | | | family member |
| | | | | | | | 3 |
| 3983 | ABLIM1 | O14639 | NM_001322882.2 | 8047 | NP_001309811.1 | 748 | actin-binding |
| | | | | | | | LIM protein 1 |
| 3983 | ABLIM1 | O14639 | NM_001352441.1 | 7493 | NP_001339370.1 | 620 | actin-binding |
| | | | | | | | LIM protein 1 |
| 3983 | ABLIM1 | O14639 | NM_001352440.1 | 7598 | NP_001339369.1 | 655 | actin-binding |
| | | | | | | | LIM protein 1 |
| 3983 | ABLIM1 | O14639 | XM_011539805.1 | 7822 | XP_011538107.1 | 696 | actin-binding |
| | | | | | | | LIM protein 1 |
| 3983 | ABLIM1 | O14639 | NM_001322885.3 | 7380 | NP_001309814.1 | 666 | actin-binding |
| | | | | | | | LIM protein 1 |
| 3983 | ABLIM1 | O14639 | NM_001322884.3 | 7386 | NP_001309813.1 | 668 | actin-binding |
| | | | | | | | LIM protein 1 |
| 3983 | ABLIM1 | O14639 | NM_001322886.3 | 7344 | NP_001309815.1 | 654 | actin-binding |
| | | | | | | | LIM protein 1 |
| 3983 | ABLIM1 | O14639 | XM_017016255.1 | 7451 | XP_016871744.1 | 703 | actin-binding |
| | | | | | | | LIM protein 1 |
| 3983 | ABLIM1 | O14639 | XM_017016250.1 | 7571 | XP_016871739.1 | 743 | actin-binding |
| | | | | | | | LIM protein 1 |
| 3983 | ABLIM1 | O14639 | XM_017016245.1 | 7655 | XP_016871734.1 | 771 | actin-binding |
| | | | | | | | LIM protein 1 |
| 3983 | ABLIM1 | O14639 | XM_006717837.2 | 7803 | XP_006717900.1 | 788 | actin-binding |
| | | | | | | | LIM protein 1 |
| 3983 | ABLIM1 | O14639 | NM_001322887.2 | 7149 | NP_001309816.1 | 636 | actin-binding |
| | | | | | | | LIM protein 1 |
| 3983 | ABLIM1 | O14639 | NM_001322883.2 | 7290 | NP_001309812.1 | 683 | actin-binding |
| | | | | | | | LIM protein 1 |
| 3983 | ABLIM1 | O14639 | NM_001003407.2 | 7395 | NP_001003407.1 | 718 | actin-binding |
| | | | | | | | LIM protein 1 |
| 3983 | ABLIM1 | O14639 | NM_002313.7 | 7560 | NP_002304.3 | 778 | actin-binding |
| | | | | | | | LIM protein 1 |
| 3983 | ABLIM1 | O14639 | XM_011539802.2 | 7586 | XP_011538104.2 | 778 | actin-binding |
| | | | | | | | LIM protein 1 |
| 3983 | ABLIM1 | O14639 | XM_024448020.1 | 7251 | XP_024303788.1 | 579 | actin-binding |
| | | | | | | | LIM protein 1 |
| 3983 | ABLIM1 | O14639 | XM_024448021.1 | 7072 | XP_024303789.1 | | actin-binding |
| | | | | | | 544 | LIM protein 1 |
| 3983 | ABLIM1 | O14639 | XM_017016256.1 | 8244 | XP_016871745.1 | | actin-binding |
| | | | | | | 669 | LIM protein 1 |
| 3983 | ABLIM1 | O14639 | XM_024448016.1 | 8469 | XP_024303784.1 | 744 | actin-binding |
| | | | | | | | LIM protein 1 |
| 3983 | ABLIM1 | O14639 | XM_024448018.1 | 8328 | XP_024303786.1 | 697 | actin-binding |
| | | | | | | | LIM protein 1 |
| 3983 | ABLIM1 | O14639 | XM_024448014.1 | 8553 | XP_024303782.1 | 772 | actin-binding |
| | | | | | | | LIM protein 1 |
| 3983 | ABLIM1 | O14639 | XM_024448013.1 | 7464 | XP_024303781.1 | 668 | actin-binding |
| | | | | | | | LIM protein 1 |
| 3983 | ABLIM1 | O14639 | XM_011539801.2 | 7548 | XP_011538103.2 | 696 | actin-binding |
| | | | | | | | LIM protein 1 |
| 3983 | ABLIM1 | O14639 | NM_001352442.2 | 7216 | NP_001339371.1 | | actin-binding |
| | | | | | | 591 | LIM protein 1 |
| 3983 | ABLIM1 | O14639 | XM_017016248.1 | 7220 | XP_016871737.1 | | actin-binding |
| | | | | | | 619 | LIM protein 1 |
| 3983 | ABLIM1 | O14639 | XM_024448017.1 | 7079 | XP_024303785.1 | 572 | actin-binding |
| | | | | | | | LIM protein 1 |
| 3983 | ABLIM1 | O14639 | XM_017016247.1 | 7220 | XP_016871736.1 | 619 | actin-binding |
| | | | | | | | LIM protein 1 |
| 3983 | ABLIM1 | O14639 | XM_024448015.1 | 7184 | XP_024303783.1 | 607 | actin-binding |
| | | | | | | | LIM protein 1 |
| 3983 | ABLIM1 | O14639 | XM_024448011.1 | 7325 | XP_024303779.1 | 654 | actin-binding |
| | | | | | | | LIM protein 1 |
| 3983 | ABLIM1 | O14639 | XM_024448012.1 | 7304 | XP_024303780.1 | 647 | actin-binding |
| | | | | | | | LIM protein 1 |
| 3983 | ABLIM1 | O14639 | XM_024448010.1 | 7445 | XP_024303778.1 | 694 | actin-binding |
| | | | | | | | LIM protein 1 |
| 3983 | ABLIM1 | O14639 | XM_024448022.1 | 7577 | XP_024303790.1 | 462 | actin-binding |
| | | | | | | | LIM protein 1 |
| 3983 | ABLIM1 | O14639 | XM_005269827.4 | 7703 | XP_005269884.1 | 504 | actin-binding |
| | | | | | | | LIM protein 1 |
| 3983 | ABLIM1 | O14639 | XM_006717846.3 | 7808 | XP_006717909.1 | 532 | actin-binding |
| | | | | | | | LIM protein 1 |
| 3983 | ABLIM1 | O14639 | XM_005269826.1 | 7073 | XP_005269883.1 | 532 | actin-binding |
| | | | | | | | LIM protein 1 |
| 3983 | ABLIM1 | O14639 | NM_001322900.2 | 6521 | NP_001309829.1 | 354 | actin-binding |
| | | | | | | | LIM protein 1 |
| 3983 | ABLIM1 | O14639 | NM_001322896.2 | 6662 | NP_001309825.1 | 401 | actin-binding |
| | | | | | | | LIM protein 1 |
| 3983 | ABLIM1 | O14639 | NM_001322897.2 | 6626 | NP_001309826.1 | 389 | actin-binding |
| | | | | | | | LIM protein 1 |
| 3983 | ABLIM1 | O14639 | NM_001322898.2 | 6599 | NP_001309827.1 | 380 | actin-binding |
| | | | | | | | LIM protein 1 |
| 3983 | ABLIM1 | O14639 | NM_001322892.2 | 6740 | NP_001309821.1 | 427 | actin-binding |
| | | | | | | | LIM protein 1 |
| 3983 | ABLIM1 | O14639 | NM_001322890.2 | 6746 | NP_001309819.1 | 429 | actin-binding |
| | | | | | | | LIM protein 1 |
| 3983 | ABLIM1 | O14639 | NM_001322895.2 | 6704 | NP_001309824.1 | 415 | actin-binding |
| | | | | | | | LIM protein 1 |
| 3983 | ABLIM1 | O14639 | NM_001352443.2 | 6851 | NP_001339372.1 | 464 | actin-binding |
| | | | | | | | LIM protein 1 |
| 3983 | ABLIM1 | O14639 | NM_001322888.2 | 6824 | NP_001309817.1 | 455 | actin-binding |
| | | | | | | | LIM protein 1 |
| 3983 | ABLIM1 | O14639 | NM_006720.4 | 6824 | NP_006711.3 | 455 | actin-binding |
| | | | | | | | LIM protein 1 |
| 3983 | ABLIM1 | O14639 | NM_001322899.2 | 6620 | NP_001309828.1 | 380 | actin-binding |
| | | | | | | | LIM protein 1 |
| 3983 | ABLIM1 | O14639 | NM_001322893.2 | 6761 | NP_001309822.1 | 427 | actin-binding |
| | | | | | | | LIM protein 1 |
| 3983 | ABLIM1 | O14639 | NM_001322891.2 | 6767 | NP_001309820.1 | 429 | actin-binding |
| | | | | | | | LIM protein 1 |
| 3983 | ABLIM1 | O14639 | NM_001322894.2 | 6725 | NP_001309823.1 | 415 | actin-binding |
| | | | | | | | LIM protein 1 |
| 3983 | ABLIM1 | O14639 | NM_001322889.2 | 6809 | NP_001309818.1 | 443 | actin-binding |
| | | | | | | | LIM protein 1 |
| 3983 | ABLIM1 | O14639 | XM_017016259.1 | 6782 | XP_016871748.1 | 464 | actin-binding |
| | | | | | | | LIM protein 1 |
| 3983 | ABLIM1 | O14639 | XM_006717847.1 | 6902 | XP_006717910.1 | 504 | actin-binding |
| | | | | | | | LIM protein 1 |
| 3983 | ABLIM1 | O14639 | XM_024448019.1 | 3079 | XP_024303787.1 | 458 | actin-binding |
| | | | | | | | LIM protein 1 |
| 3993 | LLGL2 | Q6P1M3 | XR_002957999.1 | 3837 | | | |
| 3993 | LLGL2 | Q6P1M3 | XR_001752508.1 | 3578 | | | |
| 3993 | LLGL2 | Q6P1M3 | XR_002958003.1 | 3825 | | | |
| 3993 | LLGL2 | Q6P1M3 | XR_002958000.1 | 3904 | | | |
| 3993 | LLGL2 | Q6P1M3 | XR_002958001.1 | 3947 | | | |
| 3993 | LLGL2 | Q6P1M3 | XR_002958005.1 | 3961 | | | |
| 3993 | LLGL2 | Q6P1M3 | XR_002958002.1 | 3876 | | | |
| 3993 | LLGL2 | Q6P1M3 | XR_002958004.1 | 3955 | | | |
| 3993 | LLGL2 | Q6P1M3 | XM_011524802.1 | 3531 | XP_011523104.1 | 1024 | LLGL scribble |
| | | | | | | | cell polarity |
| | | | | | | | complex |
| | | | | | | | component 2 |
| 3993 | LLGL2 | Q6P1M3 | XM_017024625.1 | 3528 | XP_016880114.1 | 1023 | LLGL scribble |
| | | | | | | | cell polarity |
| | | | | | | | complex |
| | | | | | | | component 2 |
| 3993 | LLGL2 | Q6P1M3 | XM_017024628.1 | 3488 | XP_016880117.1 | 1019 | LLGL scribble |
| | | | | | | | cell polarity |
| | | | | | | | complex |
| | | | | | | | component 2 |
| 3993 | LLGL2 | Q6P1M3 | XM_017024629.1 | 3485 | XP_016880118.1 | 1018 | LLGL scribble |
| | | | | | | | cell polarity |
| | | | | | | | complex |
| | | | | | | | component 2 |
| 3993 | LLGL2 | Q6P1M3 | XM_017024626.1 | 3519 | XP_016880115.1 | 1020 | LLGL scribble |
| | | | | | | | cell polarity |
| | | | | | | | complex |
| | | | | | | | component 2 |
| 3993 | LLGL2 | Q6P1M3 | XM_017024627.1 | 3516 | XP_016880116.1 | 1019 | LLGL scribble |
| | | | | | | | cell polarity |
| | | | | | | | complex |
| | | | | | | | component 2 |
| 3993 | LLGL2 | Q6P1M3 | XM_017024630.1 | 3476 | XP_016880119.1 | 1015 | LLGL scribble |
| | | | | | | | cell polarity |
| | | | | | | | complex |
| | | | | | | | component 2 |
| 3993 | LLGL2 | Q6P1M3 | XM_017024631.1 | 3138 | XP_016880120.1 | 999 | LLGL scribble |
| | | | | | | | cell polarity |
| | | | | | | | complex |
| | | | | | | | component 2 |
| 3993 | LLGL2 | Q6P1M3 | NM_001031803.2 | 3553 | NP_001026973.1 | 1020 | LLGL scribble |
| | | | | | | | cell polarity |
| | | | | | | | complex |
| | | | | | | | component 2 |
| 3993 | LLGL2 | Q6P1M3 | NM_004524.3 | 3510 | NP_004515.2 | 1015 | LLGL scribble |
| | | | | | | | cell polarity |
| | | | | | | | complex |
| | | | | | | | component 2 |
| 3993 | LLGL2 | Q6P1M3 | NM_001015002.2 | 1411 | NP_001015002.1 | 356 | LLGL scribble |
| | | | | | | | cell polarity |
| | | | | | | | complex |
| | | | | | | | component 2 |
| 3993 | LLGL2 | Q6P1M3 | XM_024450747.1 | 3589 | XP_024306515.1 | 356 | LLGL scribble |
| | | | | | | | cell polarity |
| | | | | | | | complex |
| | | | | | | | component 2 |
| 399665 | FAM102A | Q5T9C2 | NM_001035254.3 | 4617 | NP_001030331.1 | 384 | protein |
| | | | | | | | FAM102A |
| 399665 | FAM102A | Q5T9C2 | NM_203305.3 | 3618 | NP_976050.1 | 242 | protein |
| | | | | | | | FAM102A |
| 400720 | ZNF772 | Q68DY9 | XM_005258944.4 | 6574 | XP_005259001.1 | 377 | zinc finger |
| | | | | | | | protein 772 |
| 400720 | ZNF772 | Q68DY9 | XM_017026813.2 | 6994 | XP_016882302.1 | 393 | zinc finger |
| | | | | | | | protein 772 |
| 400720 | ZNF772 | Q68DY9 | XM_011526980.3 | 7117 | XP_011525282.1 | 434 | zinc finger |
| | | | | | | | protein 772 |
| 400720 | ZNF772 | Q68DY9 | NM_001330613.2 | 5165 | NP_001317542.1 | 377 | zinc finger |
| | | | | | | | protein 772 |
| 400720 | ZNF772 | Q68DY9 | NM_001144068.2 | 5292 | NP_001137540.1 | 448 | zinc finger |
| | | | | | | | protein 772 |
| 400720 | ZNF772 | Q68DY9 | NM_001024596.3 | 5415 | NP_001019767.1 | 489 | zinc finger |
| | | | | | | | protein 772 |
| 4050 | LTB | Q06643 | NM_009588.1 | 848 | NP_033666.1 | 77 | lymphotoxin- |
| | | | | | | | beta |
| 4050 | LTB | Q06643 | NM_002341.2 | 893 | NP_002332.1 | 244 | lymphotoxin- |
| | | | | | | | beta |
| 430 | ASCL2 | Q99929 | NM_005170.3 | 1485 | NP_005161.1 | 193 | |
| 4345 | CD200 | P41217 | NR_158642.1 | 2199 | | | |
| 4345 | CD200 | P41217 | NM_001365854.1 | 2264 | NP_001352783.1 | 262 | OX-2 |
| | | | | | | | membrane |
| | | | | | | | glycoprotein |
| 4345 | CD200 | P41217 | NM_001365853.1 | 2189 | NP_001352782.1 | 262 | OX-2 |
| | | | | | | | membrane |
| | | | | | | | glycoprotein |
| 4345 | CD200 | P41217 | NM_001365855.1 | 2184 | NP_001352784.1 | 153 | OX-2 |
| | | | | | | | membrane |
| | | | | | | | glycoprotein |
| 4345 | CD200 | P41217 | NM_001365852.1 | 2542 | NP_001352781.1 | 262 | OX-2 |
| | | | | | | | membrane |
| | | | | | | | glycoprotein |
| 4345 | CD200 | P41217 | NM_001004196.4 | 2204 | NP_001004196.2 | 294 | OX-2 |
| | | | | | | | membrane |
| | | | | | | | glycoprotein |
| 4345 | CD200 | P41217 | NM_005944.7 | 2129 | NP_005935.4 | 269 | OX-2 |
| | | | | | | | membrane |
| | | | | | | | glycoprotein |
| 4345 | CD200 | P41217 | NM_001318826.2 | 2124 | NP_001305755.1 | 153 | OX-2 |
| | | | | | | | membrane |
| | | | | | | | glycoprotein |
| 4345 | CD200 | P41217 | NM_001318828.2 | 2047 | NP_001305757.1 | 195 | OX-2 |
| | | | | | | | membrane |
| | | | | | | | glycoprotein |
| 4345 | CD200 | P41217 | NM_001318830.2 | 2042 | NP_001305759.1 | 153 | OX-2 |
| | | | | | | | membrane |
| | | | | | | | glycoprotein |
| 4345 | CD200 | P41217 | NM_001365851.2 | 1075 | NP_001352780.1 | 278 | OX-2 |
| | | | | | | | membrane |
| | | | | | | | glycoprotein |
| 439921 | MXRA7 | P84157 | NR_130926.2 | 1533 | | | |
| 439921 | MXRA7 | P84157 | NR_130928.2 | 1516 | | | |
| 439921 | MXRA7 | P84157 | NR_130927.2 | 1597 | | | |
| 439921 | MXRA7 | P84157 | NM_001008528.3 | 5671 | NP_001008528.1 | 204 | matrix- |
| | | | | | | | remodeling- |
| | | | | | | | associated |
| | | | | | | | protein 7 |
| 439921 | MXRA7 | P84157 | NM_001387278.1 | 5647 | NP_001374207.1 | 173 | matrix- |
| | | | | | | | remodeling- |
| | | | | | | | associated |
| | | | | | | | protein 7 |
| 439921 | MXRA7 | P84157 | NM_001387276.1 | 5847 | NP_001374205.1 | 247 | matrix- |
| | | | | | | | remodeling- |
| | | | | | | | associated |
| | | | | | | | protein 7 |
| 439921 | MXRA7 | P84157 | NM_198530.4 | 1833 | NP_940932.2 | 170 | matrix- |
| | | | | | | | remodeling- |
| | | | | | | | associated |
| | | | | | | | protein 7 |
| 439921 | MXRA7 | P84157 | NM_001008529.3 | 1914 | NP_001008529.1 | 177 | matrix- |
| | | | | | | | remodeling- |
| | | | | | | | associated |
| | | | | | | | protein 7 |
| 439921 | MXRA7 | P84157 | NM_001363769.2 | 2009 | NP_001350698.1 | 213 | matrix- |
| | | | | | | | remodeling- |
| | | | | | | | associated |
| | | | | | | | protein 7 |
| 439921 | MXRA7 | P84157 | NM_001387277.1 | 2090 | NP_001374206.1 | 220 | matrix- |
| | | | | | | | remodeling- |
| | | | | | | | associated |
| | | | | | | | protein 7 |
| 445815 | PALM2AKAP2 | Q8IXS6, | NM_001037293.3 | 9266 | NP_001032370.1 | 379 | paralemmin-2 |
| | | Q9Y2D5 | | | | | |
| 445815 | PALM2AKAP2 | Q8IXS6, | NM_007203.5 | 7618 | NP_009134.1 | 1103 | PALM2- |
| | | Q9Y2D5 | | | | | AKAP2 |
| | | | | | | | protein |
| 445815 | PALM2AKAP2 | Q8IXS6, | NM_147150.3 | 7579 | NP_671492.1 | 1090 | PALM2- |
| | | Q9Y2D5 | | | | | AKAP2 |
| | | | | | | | protein |
| 445815 | PALM2AKAP2 | Q8IXS6, | NM_053016.6 | 9578 | NP_443749.5 | 411 | paralemmin-2 |
| | | Q9Y2D5 | | | | | |
| 445815 | PALM2AKAP2 | Q8IXS6, | NM_001198656.1 | 7016 | NP_001185585.1 | 961 | A-kinase |
| | | Q9Y2D5 | | | | | anchor protein |
| | | | | | | | 2 |
| 445815 | PALM2AKAP2 | Q8IXS6, | NM_001004065.4 | 6977 | NP_001004065.2 | 948 | A-kinase |
| | | Q9Y2D5 | | | | | anchor protein |
| | | | | | | | 2 |
| 445815 | PALM2AKAP2 | Q8IXS6, | NM_001136562.3 | 6802 | NP_001130034.1 | 859 | A-kinase |
| | | Q9Y2D5 | | | | | anchor protein |
| | | | | | | | 2 |
| 4609 | MYC | P01106 | NM_001354870.1 | 4515 | NP_001341799.1 | 453 | myc proto- |
| | | | | | | | oncogene |
| | | | | | | | protein |
| 4609 | MYC | P01106 | NM_002467.6 | 3721 | NP_002458.2 | 454 | myc proto- |
| | | | | | | | oncogene |
| | | | | | | | protein |
| 4646 | MYO6 | Q9UM54 | NR_160538.1 | 8612 | | | |
| 4646 | MYO6 | Q9UM54 | NR_160539.1 | 2574 | | | |
| 4646 | MYO6 | Q9UM54 | XM_005248721.4 | 8637 | XP_005248778.1 | 1281 | unconventional |
| | | | | | | | myosin-VI |
| 4646 | MYO6 | Q9UM54 | XM_005248722.4 | 8622 | XP_005248779.1 | 1276 | unconventional |
| | | | | | | | myosin-VI |
| 4646 | MYO6 | Q9UM54 | XM_005248724.4 | 8610 | XP_005248781.1 | 1272 | unconventional |
| | | | | | | | myosin-VI |
| 4646 | MYO6 | Q9UM54 | XM_017010899.2 | 8583 | XP_016866388.1 | 1263 | unconventional |
| | | | | | | | myosin-VI |
| 4646 | MYO6 | Q9UM54 | NM_001368865.1 | 8642 | NP_001355794.1 | 1294 | unconventional |
| | | | | | | | myosin-VI |
| 4646 | MYO6 | Q9UM54 | NM_001368866.1 | 8615 | NP_001355795.1 | 1285 | unconventional |
| | | | | | | | myosin-VI |
| 4646 | MYO6 | Q9UM54 | NM_001368137.1 | 8576 | NP_001355066.1 | 1272 | unconventional |
| | | | | | | | myosin-VI |
| 4646 | MYO6 | Q9UM54 | NM_004999.4 | 8615 | NP_004990.3 | 1285 | unconventional |
| | | | | | | | myosin-VI |
| 4646 | MYO6 | Q9UM54 | NM_001300899.2 | 8546 | NP_001287828.1 | 1262 | unconventional |
| | | | | | | | myosin-VI |
| 4646 | MYO6 | Q9UM54 | NM_001368136.1 | 8519 | NP_001355065.1 | 1253 | unconventional |
| | | | | | | | myosin-VI |
| 4646 | MYO6 | Q9UM54 | NM_001368138.1 | 8531 | NP_001355067.1 | 1257 | unconventional |
| | | | | | | | myosin-VI |
| 4646 | MYO6 | Q9UM54 | NM_001368139.1 | 2592 | NP_001355068.1 | 281 | unconventional |
| | | | | | | | myosin-VI |
| 4646 | MYO6 | Q9UM54 | NM_001368140.1 | 2474 | NP_001355069.1 | 281 | unconventional |
| | | | | | | | myosin-VI |
| 4646 | MYO6 | Q9UM54 | XM_024446447.1 | 8540 | XP_024302215.1 | 1294 | unconventional |
| | | | | | | | myosin-VI |
| 4753 | NELL2 | Q99435 | NM_006159.2 | 3312 | NP_006150.1 | 816 | protein kinase |
| | | | | | | | C-binding |
| | | | | | | | protein NELL2 |
| 4753 | NELL2 | Q99435 | XM_011538396.1 | 3260 | XP_011536698.1 | 816 | protein kinase |
| | | | | | | | C-binding |
| | | | | | | | protein NEI.I.2 |
| 4753 | NELL2 | Q99435 | XM_017019344.1 | 3275 | XP_016874833.1 | 821 | protein kinase |
| | | | | | | | C-binding |
| | | | | | | | protein NELL2 |
| 4753 | NELL2 | Q99435 | XM_005268905.3 | 3464 | XP_005268962.1 | 816 | protein kinase |
| | | | | | | | C-binding |
| | | | | | | | protein NELL2 |
| 4753 | NELL2 | Q99435 | XM_017019343.1 | 3479 | XP_016874832.1 | 821 | protein kinase |
| | | | | | | | C-binding |
| | | | | | | | protein NELL2 |
| 4753 | NELL2 | Q99435 | XM_017019341.2 | 3397 | XP_016874830.1 | 871 | protein kinase |
| | | | | | | | C-binding |
| | | | | | | | protein NELL2 |
| 4753 | NELL2 | Q99435 | XM_017019342.2 | 3237 | XP_016874831.1 | 821 | protein kinase |
| | | | | | | | C-binding |
| | | | | | | | protein NELL2 |
| 4753 | NELL2 | Q99435 | NM_001145110.2 | 3329 | NP_001138582.1 | 839 | protein kinase |
| | | | | | | | C-binding |
| | | | | | | | protein NELL2 |
| 4753 | NELL2 | Q99435 | NM_001145107.2 | 3407 | NP_001138579.1 | 866 | protein kinase |
| | | | | | | | C-binding |
| | | | | | | | protein NELL2 |
| 4753 | NELL2 | Q99435 | NM_001145108.2 | 3553 | NP_001138580.1 | 816 | protein kinase |
| | | | | | | | C-binding |
| | | | | | | | protein NELL2 |
| 4753 | NELL2 | Q99435 | NM_001145109.2 | 3162 | NP_001138581.1 | 815 | protein kinase |
| | | | | | | | C-binding |
| | | | | | | | protein NELL2 |
| 4818 | NKG7 | Q16617 | XM_006723228.3 | 665 | XP_006723291.1 | 116 | protein NKG7 |
| 4818 | NKG7 | Q16617 | XM_005258955.3 | 707 | XP_005259012.1 | 130 | protein NKG7 |
| 4818 | NKG7 | Q16617 | NM_001363693.2 | 755 | NP_001350622.1 | 142 | protein NKG7 |
| 4818 | NKG7 | Q16617 | NM_005601.4 | 774 | NP_005592.1 | 165 | protein NKG7 |
| 4907 | NT5E | P21589 | NM_002526.4 | 3562 | NP_002517.1 | 574 | 5′-nucleotidase |
| 4907 | NT5E | P21589 | NM_001204813.2 | 3412 | NP_001191742.1 | 524 | 5′-nucleotidase |
| 5026 | P2RX5 | Q93086 | NM_175080.3 | 1985 | NP_778255.1 | 397 | P2X |
| | | | | | | | purinoceptor 5 |
| 5026 | P2RX5 | Q93086 | NM_001204520.2 | 1988 | NP_001191449.1 | 398 | P2X |
| | | | | | | | purinoceptor 5 |
| 5026 | P2RX5 | Q93086 | NM_001204519.2 | 2057 | NP_001191448.1 | 421 | P2X |
| | | | | | | | purinoceptor 5 |
| 5026 | P2RX5 | Q93086 | NM_002561.4 | 2060 | NP_002552.2 | 422 | P2X |
| | | | | | | | purinoceptor 5 |
| 50854 | SNHG32 | | NR_160948.1 | 749 | | | |
| 50854 | SNHG32 | | NR_160946.1 | 850 | | | |
| 50854 | SNHG32 | | NR_160949.1 | 839 | | | |
| 50854 | SNHG32 | | NR_160953.1 | 745 | | | |
| 50854 | SNHG32 | | NR_160945.1 | 704 | | | |
| 50854 | SNHG32 | | NR_160951.1 | 696 | | | |
| 50854 | SNHG32 | | NR_160947.1 | 693 | | | |
| 50854 | SNHG32 | | NR_160952.1 | 602 | | | |
| 50854 | SNHG32 | | NR_160950.1 | 490 | | | |
| 51070 | NOSIP | Q9Y314 | NM_001270960.2 | 1232 | NP_001257889.1 | 301 | nitric oxide |
| | | | | | | | synthase- |
| | | | | | | | interacting |
| | | | | | | | protein |
| 51070 | NOSIP | Q9Y314 | NM_015953.5 | 1351 | NP_057037.1 | 301 | nitric oxide |
| | | | | | | | synthase- |
| | | | | | | | interacting |
| | | | | | | | protein |
| 51070 | NOSIP | Q9Y314 | NM_001363649.2 | 1360 | NP_001350578.1 | 304 | nitric oxide |
| | | | | | | | synthase- |
| | | | | | | | interacting |
| | | | | | | | protein |
| 51070 | NOSIP | Q9Y314 | XM_024451529.1 | 1127 | XP_024307297.1 | 347 | nitric oxide |
| | | | | | | | synthase- |
| | | | | | | | interacting |
| | | | | | | | protein |
| 51070 | NOSIP | Q9Y314 | XM_005258964.5 | 997 | XP_005259021.1 | 304 | nitric oxide |
| | | | | | | | synthase- |
| | | | | | | | interacting |
| | | | | | | | protein |
| 51070 | NOSIP | Q9Y314 | XM_017026851.1 | 1099 | XP_016882340.1 | 301 | nitric oxide |
| | | | | | | | synthase- |
| | | | | | | | interacting |
| | | | | | | | protein |
| 51070 | NOSIP | Q9Y314 | XM_024451530.1 | 1204 | XP_024307298.1 | 347 | nitric oxide |
| | | | | | | | synthase- |
| | | | | | | | interacting |
| | | | | | | | protein |
| 51070 | NOSIP | Q9Y314 | XM_017026852.1 | 1080 | XP_016882341.1 | 301 | nitric oxide |
| | | | | | | | synthase- |
| | | | | | | | interacting |
| | | | | | | | protein |
| 51070 | NOSIP | Q9Y314 | XM_011527017.2 | 1081 | XP_011525319.1 | 302 | nitric oxide |
| | | | | | | | synthase- |
| | | | | | | | interacting |
| | | | | | | | protein |
| 51070 | NOSIP | Q9Y314 | XM_024451528.1 | 1214 | XP_024307296.1 | 348 | nitric oxide |
| | | | | | | | synthase- |
| | | | | | | | interacting |
| | | | | | | | protein |
| 51070 | NOSIP | Q9Y314 | XM_011527015.2 | 1049 | XP_011525317.1 | 305 | nitric oxide |
| | | | | | | | synthase- |
| | | | | | | | interacting |
| | | | | | | | protein |
| 51176 | LEF1 | Q9UJU2 | NM_001130714.3 | 3450 | NP_001124186.1 | 386 | lymphoid |
| | | | | | | | enhancer- |
| | | | | | | | binding factor |
| | | | | | | | 1 |
| 51176 | LEF1 | Q9UJU2 | NM_001130713.3 | 3491 | NP_001124185.1 | 371 | lymphoid |
| | | | | | | | enhancer- |
| | | | | | | | binding factor |
| | | | | | | | 1 |
| 51176 | LEF1 | Q9UJU2 | NM_016269.5 | 3575 | NP_057353.1 | 399 | lymphoid |
| | | | | | | | enhancer- |
| | | | | | | | binding factor |
| | | | | | | | 1 |
| 51176 | LEF1 | Q9UJU2 | XM_005263046.3 | 2562 | XP_005263103.1 | 414 | lymphoid |
| | | | | | | | enhancer- |
| | | | | | | | binding factor |
| | | | | | | | 1 |
| 51176 | LEF1 | Q9UJU2 | XM_006714233.1 | 2263 | XP_006714296.1 | 299 | lymphoid |
| | | | | | | | enhancer- |
| | | | | | | | binding factor |
| | | | | | | | 1 |
| 51176 | LEF1 | Q9UJU2 | XM_005263048.1 | 2102 | XP_005263105.1 | 318 | lymphoid |
| | | | | | | | enhancer- |
| | | | | | | | binding factor |
| | | | | | | | 1 |
| 51176 | LEF1 | Q9UJU2 | NM_001166119.2 | 2143 | NP_001159591.1 | 303 | lymphoid |
| | | | | | | | enhancer- |
| | | | | | | | binding factor |
| | | | | | | | 1 |
| 51176 | LEF1 | Q9UJU2 | XM_005263047.1 | 2186 | XP_005263104.1 | 346 | lymphoid |
| | | | | | | | enhancer- |
| | | | | | | | binding factor |
| | | | | | | | 1 |
| 51361 | HOOK1 | Q9UJC3 | XR_946665.1 | 5670 | | | |
| 51361 | HOOK1 | Q9UJC3 | XR_246271.1 | 2325 | | | |
| 51361 | HOOK1 | Q9UJC3 | XM_011541563.1 | 1925 | XP_011539865.1 | 559 | protein Hook |
| | | | | | | | homolog 1 |
| 51361 | HOOK1 | Q9UJC3 | XM_006710676.1 | 1902 | XP_006710739.1 | 560 | protein Hook |
| | | | | | | | homolog 1 |
| 51361 | HOOK1 | Q9UJC3 | XM_017001424.1 | 2067 | XP_016856913.1 | 451 | protein Hook |
| | | | | | | | homolog 1 |
| 51361 | HOOK1 | Q9UJC3 | NM_015888.6 | 5713 | NP_056972.1 | 728 | |
| 51361 | HOOK1 | Q9UJC3 | XM_011541562.2 | 3775 | XP_011539864.1 | 686 | protein Hook |
| | | | | | | | homolog 1 |
| 51361 | HOOK1 | Q9UJC3 | XM_024447520.1 | 4374 | XP_024303288.1 | 686 | protein Hook |
| | | | | | | | homolog 1 |
| 51429 | SNX9 | Q9Y5X1 | XM_005267015.2 | 3793 | XP_005267072.1 | 588 | sorting nexin-9 |
| 51429 | SNX9 | Q9Y5X1 | NM_016224.5 | 4216 | NP_057308.1 | 595 | |
| 51429 | SNX9 | Q9Y5X1 | XM_011535886.3 | 2046 | XP_011534188.1 | 501 | sorting nexin-9 |
| 5324 | PLAG1 | Q6DJT9 | NM_001114635.2 | 6847 | NP_001108107.1 | 418 | zinc finger |
| | | | | | | | protein PLAG1 |
| 5324 | PLAG1 | Q6DJT9 | NM_001114634.2 | 7206 | NP_001108106.1 | 500 | zinc finger |
| | | | | | | | protein PLAG1 |
| 5324 | PLAG1 | Q6DJT9 | NM_002655.3 | 7311 | NP_002646.2 | 500 | zinc finger |
| | | | | | | | protein PLAG1 |
| 5324 | PLAG1 | Q6DJT9 | XM_017013577.1 | 6758 | XP_016869066.1 | 418 | zinc finger |
| | | | | | | | protein PLAG1 |
| 5324 | PLAG1 | Q6DJT9 | XM_011517544.2 | 6799 | XP_011515846.1 | 418 | zinc finger |
| | | | | | | | protein PLAG1 |
| 5324 | PLAG1 | Q6DJT9 | XM_017013576.1 | 7241 | XP_016869065.1 | 500 | zinc finger |
| | | | | | | | protein PLAG1 |
| 53335 | BCL11A | Q9H165 | NM_1385592. | 3161 | NP_612569.1 | 243 | B-cell |
| | | | | | | | lymphoma/ |
| | | | | | | | leukemia 11A |
| 53335 | BCL11A | Q9H165 | NM_018014.4 | 4761 | NP_060484.2 | 773 | B-cell |
| | | | | | | | lymphoma/ |
| | | | | | | | leukemia 11A |
| 53335 | BCL11A | Q9H165 | NM_001363864.1 | 4008 | NP_001350793.1 | 793 | B-cell |
| | | | | | | | lymphoma/ |
| | | | | | | | leukemia 11A |
| 53335 | BCL11A | Q9H165 | XM_011532910.1 | 3119 | XP_011531212.1 | 827 | B-cell |
| | | | | | | | lymphoma/ |
| | | | | | | | leukemia 11A |
| 53335 | BCL11A | Q9H165 | XM_011532909.1 | 3247 | XP_011531211.1 | 835 | B-cell |
| | | | | | | | lymphoma/ |
| | | | | | | | leukemia 11A |
| 53335 | BCL11A | Q9H165 | NM_022893.4 | 6102 | NP_075044.2 | 835 | B-cell |
| | | | | | | | lymphoma/ |
| | | | | | | | leukemia 11A |
| 53335 | BCL11A | Q9H165 | NM_001365609.1 | 5844 | NP_001352538.1 | 801 | B-cell |
| | | | | | | | lymphoma/ |
| | | | | | | | leukemia 11A |
| 53335 | BCL11A | Q9H165 | XM_024452962.1 | 5925 | XP_024308730.1 | 783 | B-cell |
| | | | | | | | lymphoma/ |
| | | | | | | | leukemia 11A |
| 53335 | BCL11A | Q9H165 | XM_017004335.1 | 5654 | XP_016859824.1 | 799 | B-cell |
| | | | | | | | lymphoma/ |
| | | | | | | | leukemia 11A |
| 53335 | BCL11A | Q9H165 | XM_017004333.1 | 5714 | XP_016859822.1 | 833 | B-cell |
| | | | | | | | lymphoma/ |
| | | | | | | | leukemia 11A |
| 53335 | BCL11A | Q9H165 | XM_024452963.1 | 6100 | XP_024308731.1 | 783 | B-cell |
| | | | | | | | lymphoma/ |
| | | | | | | | leukemia 11A |
| 53335 | BCL11A | Q9H165 | XM_017004336.1 | 5509 | XP_016859825.1 | 724 | B-cell |
| | | | | | | | lymphoma/ |
| | | | | | | | leukemia 11A |
| 54463 | RETREG1 | Q9H6L5 | XM_011514053.3 | 3442 | XP_011512355.1 | 537 | reticulophagy |
| | | | | | | | regulator 1 |
| 54463 | RETREG1 | Q9H6L5 | XM_011514055.3 | 3086 | XP_011512357.1 | 364 | reticulophagy |
| | | | | | | | regulator 1 |
| 54463 | RETREG1 | Q9H6L5 | XM_024446118.1 | 2858 | XP_024301886.1 | 322 | reticulophagy |
| | | | | | | | regulator 1 |
| 54463 | RETREG1 | Q9H6L5 | XM_011514054.2 | 3294 | XP_011512356.1 | 396 | reticulophagy |
| | | | | | | | regulator 1 |
| 54463 | RETREG1 | Q9H6L5 | XM_024446117.1 | 2866 | XP_024301885.1 | 322 | reticulophagy |
| | | | | | | | regulator 1 |
| 54463 | RETREG1 | Q9H6L5 | NM_001034850.3 | 3208 | NP_001030022.1 | 497 | reticulophagy |
| | | | | | | | regulator 1 |
| 54463 | RETREG1 | Q9H6L5 | NM_019000.5 | 3145 | NP_061873.2 | 356 | reticulophagy |
| | | | | | | | regulator 1 |
| 54674 | LRRN3 | Q9H3W5 | NM_001099660.2 | 3607 | NP_001093130.1 | 708 | |
| 54674 | LRRN3 | Q9H3W5 | NM_001099658.2 | 3500 | NP_001093128.1 | 708 | |
| 54674 | LRRN3 | Q9H3W5 | NM_018334.5 | 3418 | NP_060804.3 | 708 | |
| 55020 | TTC38 | Q5R3I4 | XR_002958710.1 | 4363 | | | |
| 55020 | TTC38 | Q5R3I4 | XR_001755259.2 | 2771 | | | |
| 55020 | TTC38 | Q5R3I4 | XR_001755258.2 | 2865 | | | |
| 55020 | TTC38 | Q5R3I4 | XR_244380.4 | 2716 | | | |
| 55020 | TTC38 | Q5R3I4 | XR_001755260.2 | 1667 | | | |
| 55020 | TTC38 | Q5R3I4 | XR_001755257.1 | 2843 | | | |
| 55020 | TTC38 | Q5R3I4 | XM_011530259.1 | 2738 | XP_011528561.1 | 404 | tetratricopeptide |
| | | | | | | | repeat protein |
| | | | | | | | 38 |
| 55020 | TTC38 | Q5R3I4 | XM_011530260.3 | 2195 | XP_011528562.1 | 307 | tetratricopeptide |
| | | | | | | | repeat protein |
| | | | | | | | 38 |
| 55020 | TTC38 | Q5R3I4 | NM_017931.4 | 2566 | NP_060401.3 | 469 | |
| 55342 | STRBP | Q96SI9 | XR_001746347.1 | 8827 | | | |
| 55342 | STRBP | Q96SI9 | XR_001746346.1 | 8938 | | | |
| 55342 | STRBP | Q96SI9 | XR_001746349.1 | 2582 | | | |
| 55342 | STRBP | Q96SI9 | XR_001746348.1 | 2613 | | | |
| 55342 | STRBP | Q96SI9 | NR_033234.2 | 6376 | | | |
| 55342 | STRBP | Q96SI9 | NM_001376109.1 | 3315 | NP_001363038.1 | 648 | spermatid |
| | | | | | | | perinuclear |
| | | | | | | | RNA-binding |
| | | | | | | | protein |
| 55342 | STRBP | Q96SI9 | XM_017014898.1 | 3257 | XP_016870387.1 | 648 | spermatid |
| | | | | | | | perinuclear |
| | | | | | | | RNA-binding |
| | | | | | | | protein |
| 55342 | STRBP | Q96SI9 | NM_001171137.2 | 6422 | NP_001164608.1 | 658 | spermatid |
| | | | | | | | perinuclear |
| | | | | | | | RNA-binding |
| | | | | | | | protein |
| 55342 | STRBP | Q96SI9 | NM_018387.5 | 6451 | NP_060857.2 | 672 | spermatid |
| | | | | | | | perinuclear |
| | | | | | | | RNA-binding |
| | | | | | | | protein |
| 55342 | STRBP | Q96SI9 | NM_001376106.1 | 3223 | NP_001363035.1 | 672 | spermatid |
| | | | | | | | perinuclear |
| | | | | | | | RNA-binding |
| | | | | | | | protein |
| 55342 | STRBP | Q96SI9 | NM_001376107.1 | 3677 | NP_001363036.1 | 672 | spermatid |
| | | | | | | | perinuclear |
| | | | | | | | RNA-binding |
| | | | | | | | protein |
| 55342 | STRBP | Q96SI9 | XM_017014891.2 | 5752 | XP_016870380.1 | 674 | spermatid |
| | | | | | | | perinuclear |
| | | | | | | | RNA-binding |
| | | | | | | | protein |
| 55342 | STRBP | Q96SI9 | XM_024447605.1 | 3250 | XP_024303373.1 | 490 | spermatid |
| | | | | | | | perinuclear |
| | | | | | | | RNA-binding |
| | | | | | | | protein |
| 55342 | STRBP | Q96SI9 | XM_017014897.1 | 3120 | XP_016870386.1 | 657 | spermatid |
| | | | | | | | perinuclear |
| | | | | | | | RNA-binding |
| | | | | | | | protein |
| 55342 | STRBP | Q96SI9 | XM_017014890.1 | 5705 | XP_016870379.1 | 674 | spermatid |
| | | | | | | | perinuclear |
| | | | | | | | RNA-binding |
| | | | | | | | protein |
| 55342 | STRBP | Q96SI9 | XM_017014892.1 | 5937 | XP_016870381.1 | 674 | spermatid |
| | | | | | | | perinuclear |
| | | | | | | | RNA-binding |
| | | | | | | | protein |
| 55342 | STRBP | Q96SI9 | XM_024447604.1 | 5001 | XP_024303372.1 | 492 | spermatid |
| | | | | | | | perinuclear |
| | | | | | | | RNA-binding |
| | | | | | | | protein |
| 55501 | CHST12 | Q9NRB3 | XR_002956463.1 | 3859 | | | |
| 55501 | CHST12 | Q9NRB3 | XM_011515444.2 | 1536 | XP_011513746.1 | 414 | carbohydrate |
| | | | | | | | sulfotransferase |
| | | | | | | | 12 |
| 55501 | CHST12 | Q9NRB3 | NM_018641.5 | 15985 | NP_061111.1 | | |
| | | | | | | 414 | |
| 55501 | CHST12 | Q9NRB3 | NM_001243795.2 | 15971 | NP_001230724.1 | 414 | |
| 55501 | CHST12 | Q9NRB3 | NM_001243794.2 | 15957 | NP_001230723.1 | 414 | |
| 55501 | CHST12 | Q9NRB3 | XM_011515443.2 | 1713 | XP_011513745.1 | 414 | carbohydrate |
| | | | | | | | sulfotransferase |
| | | | | | | | 12 |
| 55536 | CDCA7L | Q96GN5 | NM_001127371.3 | 2745 | NP_001120843.1 | 408 | cell division |
| | | | | | | | cycle- |
| | | | | | | | associated 7- |
| | | | | | | | like protein |
| 55536 | CDCA7L | Q96GN5 | NM_018719.5 | 2883 | NP_061189.2 | 454 | cell division |
| | | | | | | | cycle- |
| | | | | | | | associated 7- |
| | | | | | | | like protein |
| 55536 | CDCA7L | Q96GN5 | NM_001127370.3 | 2937 | NP_001120842.1 | 420 | cell division |
| | | | | | | | cycle- |
| | | | | | | | associated 7- |
| | | | | | | | like protein |
| 56895 | AGPAT4 | Q9NRZ5 | XR_001743516.1 | 7876 | | | |
| 56895 | AGPAT4 | Q9NRZ5 | NM_020133.3 | 7923 | NP_064518.1 | 378 | |
| 56895 | AGPAT4 | Q9NRZ5 | XM_017011059.1 | 8094 | XP_016866548.1 | 244 | 1-acyl-sn- |
| | | | | | | | glycerol-3- |
| | | | | | | | phosphate |
| | | | | | | | acyltransferase |
| | | | | | | | delta |
| 56895 | AGPAT4 | Q9NRZ5 | XM_005267053.3 | 7740 | XP_005267110.1 | 244 | 1-acyl-sn- |
| | | | | | | | glycerol-3- |
| | | | | | | | phosphate |
| | | | | | | | acyltransferase |
| | | | | | | | delta |
| 56895 | AGPAT4 | Q9NRZ5 | XM_006715514.3 | 7748 | XP_006715577.1 | 324 | 1-acyl-sn- |
| | | | | | | | glycerol-3- |
| | | | | | | | phosphate |
| | | | | | | | acyltransferase |
| | | | | | | | delta |
| 56937 | PMEPA1 | Q969W9 | NM_199171.3 | 4561 | NP_954640.1 | 237 | protein |
| | | | | | | | TMEPAI |
| 56937 | PMEPA1 | Q969W9 | NM_199170.3 | 4523 | NP_954639.1 | 237 | protein |
| | | | | | | | TMEPAI |
| 56937 | PMEPA1 | Q969W9 | NM_199169.3 | 4530 | NP_954638.1 | 252 | protein |
| | | | | | | | TMEPAI |
| 56937 | PMEPA1 | Q969W9 | NM_020182.5 | 4954 | NP_064567.2 | 287 | protein |
| | | | | | | | TMEPAI |
| 56937 | PMEPA1 | Q969W9 | NM_001255976.2 | 4705 | NP_001242905.1 | 259 | protein |
| | | | | | | | TMEPAI |
| 57136 | APMAP | Q9HDC9 | XM_005260763.3 | 2188 | XP_005260820.1 | 289 | adipocyte |
| | | | | | | | plasma |
| | | | | | | | membrane- |
| | | | | | | | associated |
| | | | | | | | protein |
| 57136 | APMAP | Q9HDC9 | NM_020531.3 | 2202 | NP_065392.1 | 416 | |
| 5796 | PTPRK | Q15262 | NM_001291984.2 | 6003 | NP_001278913.1 | 1439 | receptor-type |
| | | | | | | | tyrosine- |
| | | | | | | | protein |
| | | | | | | | phosphatase |
| | | | | | | | kappa |
| 5796 | PTPRK | Q15262 | NM_002844.4 | 6006 | NP_002835.2 | 1440 | receptor-type |
| | | | | | | | tyrosine- |
| | | | | | | | protein |
| | | | | | | | phosphatase |
| | | | | | | | kappa |
| 5796 | PTPRK | Q15262 | NM_001135648.3 | 6024 | NP_001129120.1 | 1446 | receptor-type |
| | | | | | | | tyrosine- |
| | | | | | | | protein |
| | | | | | | | phosphatase |
| | | | | | | | kappa |
| 5796 | PTPRK | Q15262 | NM_001291981.2 | 6072 | NP_001278910.1 | 1462 | receptor-type |
| | | | | | | | tyrosine- |
| | | | | | | | protein |
| | | | | | | | phosphatase |
| | | | | | | | kappa |
| 5796 | PTPRK | Q15262 | XM_006715537.3 | 5993 | XP_006715600.1 | 1445 | receptor-type |
| | | | | | | | tyrosine- |
| | | | | | | | protein |
| | | | | | | | phosphatase |
| | | | | | | | kappa |
| 5796 | PTPRK | Q15262 | XM_011536014.3 | 6041 | XP_011534316.1 | 1461 | receptor-type |
| | | | | | | | tyrosine- |
| | | | | | | | protein |
| | | | | | | | phosphatase |
| | | | | | | | kappa |
| 5796 | PTPRK | Q15262 | XM_011536015.3 | 6032 | XP_011534317.1 | 1458 | receptor-type |
| | | | | | | | tyrosine- |
| | | | | | | | protein |
| | | | | | | | phosphatase |
| | | | | | | | kappa |
| 5796 | PTPRK | Q15262 | XM_011536016.3 | 6022 | XP_011534318.1 | 1456 | receptor-type |
| | | | | | | | tyrosine- |
| | | | | | | | protein |
| | | | | | | | phosphatase |
| | | | | | | | kappa |
| 5796 | PTPRK | Q15262 | XM_017011146.1 | 6031 | XP_016866635.1 | 1375 | receptor-type |
| | | | | | | | tyrosine- |
| | | | | | | | protein |
| | | | | | | | phosphatase |
| | | | | | | | kappa |
| 5796 | PTPRK | Q15262 | XM_011536018.2 | 6079 | XP_011534320.1 | 1391 | receptor-type |
| | | | | | | | tyrosine- |
| | | | | | | | protein |
| | | | | | | | phosphatase |
| | | | | | | | kappa |
| 5796 | PTPRK | Q15262 | XM_017011147.1 | 6009 | XP_016866636.1 | 1374 | receptor-type |
| | | | | | | | tyrosine- |
| | | | | | | | protein |
| | | | | | | | phosphatase |
| | | | | | | | kappa |
| 5796 | PTPRK | Q15262 | XM_017011148.1 | 5988 | XP_016866637.1 | 1368 | receptor-type |
| | | | | | | | tyrosine- |
| | | | | | | | protein |
| | | | | | | | phosphatase |
| | | | | | | | kappa |
| 5796 | PTPRK | Q15262 | XM_017011145.1 | 6082 | XP_016866634.1 | 1375 | receptor-type |
| | | | | | | | tyrosine- |
| | | | | | | | protein |
| | | | | | | | phosphatase |
| | | | | | | | kappa |
| 5796 | PTPRK | Q15262 | XM_011536017.3 | 5945 | XP_011534319.1 | 1391 | receptor-type |
| | | | | | | | tyrosine- |
| | | | | | | | protein |
| | | | | | | | phosphatase |
| | | | | | | | kappa |
| 5796 | PTPRK | Q15262 | XM_017011149.2 | 3666 | XP_016866638.1 | 727 | receptor-type |
| | | | | | | | tyrosine- |
| | | | | | | | protein |
| | | | | | | | phosphatase |
| | | | | | | | kappa |
| 5796 | PTPRK | Q15262 | XM_017011150.2 | 3653 | XP_016866639.1 | 723 | receptor-type |
| | | | | | | | tyrosine- |
| | | | | | | | protein |
| | | | | | | | phosphatase |
| | | | | | | | kappa |
| 5796 | PTPRK | Q15262 | NM_001291983.2 | 2932 | NP_001278912.1 | 761 | receptor-type |
| | | | | | | | tyrosine- |
| | | | | | | | protein |
| | | | | | | | phosphatase |
| | | | | | | | kappa |
| 5796 | PTPRK | Q15262 | NM_001291982.2 | 3162 | NP_001278911.1 | 876 | receptor-type |
| | | | | | | | tyrosine- |
| | | | | | | | protein |
| | | | | | | | phosphatase |
| | | | | | | | kappa |
| 5796 | PTPRK | Q15262 | XM_011536020.3 | 3970 | XP_011534322.1 | 747 | receptor-type |
| | | | | | | | tyrosine- |
| | | | | | | | protein |
| | | | | | | | phosphatase |
| | | | | | | | kappa |
| 5796 | PTPRK | Q15262 | XM_011536021.3 | 2519 | XP_011534323.1 | 736 | receptor-type |
| | | | | | | | tyrosine- |
| | | | | | | | protein |
| | | | | | | | phosphatase |
| | | | | | | | kappa |
| 60468 | BACH2 | Q9BYV9 | NM_001170794.2 | 9044 | NP_001164265.1 | 84 | |
| 60468 | BACH2 | Q9BYV9 | NM_021813.4 | 9215 | NP_068585.1 | 841 | |
| 60468 | BACH2 | Q9BYV9 | XM_017011166.2 | 9014 | XP_016866655.1 | 841 | transcription |
| | | | | | | | regulator |
| | | | | | | | protein |
| | | | | | | | BACH2 |
| 60468 | BACH2 | Q9BYV9 | XM_024446511.1 | 9837 | XP_024302279.1 | 927 | transcription |
| | | | | | | | regulator |
| | | | | | | | protein |
| | | | | | | | BACH2 |
| 60468 | BACH2 | Q9BYV9 | XM_024446510.1 | 8971 | XP_024302278.1 | 927 | transcription |
| | | | | | | | regulator |
| | | | | | | | protein |
| | | | | | | | BACH2 |
| 60468 | BACH2 | Q9BYV9 | XM_005248759.5 | 15454 | XP_005248816.1 | 841 | transcription |
| | | | | | | | regulator |
| | | | | | | | protein |
| | | | | | | | BACH2 |
| 60468 | BACH2 | Q9BYV9 | XM_005248758.5 | 15534 | XP_005248815.1 | 877 | transcription |
| | | | | | | | regulator |
| | | | | | | | protein |
| | | | | | | | BACH2 |
| 60468 | BACH2 | Q9BYV9 | XM_011536039.3 | 3742 | XP_011534341.1 | 841 | transcription |
| | | | | | | | regulator |
| | | | | | | | protein |
| | | | | | | | BACH2 |
| 60468 | BACH2 | Q9BYV9 | XM_024446513.1 | 3616 | XP_024302281.1 | 841 | transcription |
| | | | | | | | regulator |
| | | | | | | | protein |
| | | | | | | | BACH2 |
| 60468 | BACH2 | Q9BYV9 | XM_011536040.2 | 4043 | XP_011534342.1 | 841 | transcription |
| | | | | | | | regulator |
| | | | | | | | protein |
| | | | | | | | BACH2 |
| 60468 | BACH2 | Q9BYV9 | XM_017011167.2 | 3464 | XP_016866656.1 | 708 | transcription |
| | | | | | | | regulator |
| | | | | | | | protein |
| | | | | | | | BACH2 |
| 6136 | RPL12 | P30050 | NM_000976.4 | 634 | NP_000967.1 | 165 | |
| 6152 | RPL24 | P83731 | NM_000986.4 | 560 | NP_000977.1 | 157 | |
| 6193 | RPS5 | P46782 | NM_001009.4 | 741 | NP_001000.2 | 204 | |
| 6204 | RPS10 | P46783 | NM_001014.5 | 588 | NP_001005.1 | 165 | |
| 6204 | RPS10 | P46783 | NM_001204091.2 | 592 | NP_001191020.1 | 165 | |
| 6204 | RPS10 | P46783 | NM_001203245.3 | 824 | NP_001190174.1 | 165 | |
| 6207 | RPS13 | P62277 | NM_001017.3 | 527 | NP_001008.1 | 151 | |
| 6322 | SCML1 | Q9UN30 | XM_005274578.2 | 2926 | XP_005274635.1 | 330 | sex comb on |
| | | | | | | | midleg-like |
| | | | | | | | protein 1 |
| 6322 | SCML1 | Q9UN30 | XM_005274579.2 | 2839 | XP_005274636.1 | 301 | sex comb on |
| | | | | | | | midleg-like |
| | | | | | | | protein 1 |
| 6322 | SCML1 | Q9UN30 | NM_001037540.3 | 2887 | NP_001032629.1 | 329 | sex comb on |
| | | | | | | | midleg-like |
| | | | | | | | protein 1 |
| 6322 | SCML1 | Q9UN30 | NM_006746.6 | 2806 | NP_006737.2 | 302 | sex comb on |
| | | | | | | | midleg-like |
| | | | | | | | protein 1 |
| 6322 | SCML1 | Q9UN30 | NM_001037535.3 | 2657 | NP_001032624.1 | 208 | sex comb on |
| | | | | | | | midleg-like |
| | | | | | | | protein 1 |
| 6322 | SCML1 | Q9UN30 | NM_001037536.3 | 2654 | NP_001032625.1 | 208 | sex comb on |
| | | | | | | | midleg-like |
| | | | | | | | protein 1 |
| 6322 | SCML1 | Q9UN30 | XM_011545564.3 | 8925 | XP_011543866.1 | 330 | sex comb on |
| | | | | | | | midleg-like |
| | | | | | | | protein 1 |
| 6322 | SCML1 | Q9UN30 | XM_006724508.4 | 8922 | XP_006724571.1 | 329 | sex comb on |
| | | | | | | | midleg-like |
| | | | | | | | protein 1 |
| 6322 | SCML1 | Q9UN30 | XM_017029723.2 | 8841 | XP_016885212.1 | 302 | sex comb on |
| | | | | | | | midleg-like |
| | | | | | | | protein 1 |
| 6322 | SCML1 | Q9UN30 | XM_006724509.4 | 8838 | XP_006724572.1 | 301 | sex comb on |
| | | | | | | | midleg-like |
| | | | | | | | protein 1 |
| 64784 | CRTC3 | Q6UUV7 | XM_005254968.2 | 5219 | XP_005255025.1 | 618 | CREB- |
| | | | | | | | regulated |
| | | | | | | | transcription |
| | | | | | | | coactivator 3 |
| 64784 | CRTC3 | Q6UUV7 | XM_011521906.2 | 5123 | XP_011520208.1 | 586 | CREB- |
| | | | | | | | regulated |
| | | | | | | | transcription |
| | | | | | | | coactivator 3 |
| 64784 | CRTC3 | Q6UUV7 | NM_022769.5 | 5214 | NP_073606.3 | 619 | CREB- |
| | | | | | | | regulated |
| | | | | | | | transcription |
| | | | | | | | coactivator 3 |
| 64784 | CRTC3 | Q6UUV7 | NM_001042574.3 | 5211 | NP_001036039.1 | 618 | CREB- |
| | | | | | | | regulated |
| | | | | | | | transcription |
| | | | | | | | coactivator 3 |
| 64784 | CRTC3 | Q6UUV7 | XM_024450019.1 | 5175 | XP_024305787.1 | 428 | CREB- |
| | | | | | | | regulated |
| | | | | | | | transcription |
| | | | | | | | coactivator 3 |
| 64784 | CRTC3 | Q6UUV7 | XM_024450018.1 | 5976 | XP_024305786.1 | 428 | CREB- |
| | | | | | | | regulated |
| | | | | | | | transcription |
| | | | | | | | coactivator 3 |
| 65982 | ZSCAN18 | Q8TBC5 | NR_027135.2 | 1756 | | | |
| 65982 | ZSCAN18 | Q8TBC5 | XM_006723335.2 | 2634 | XP_006723398.1 | 510 | zinc finger and |
| | | | | | | | SCAN |
| | | | | | | | domain- |
| | | | | | | | containing |
| | | | | | | | protein 18 |
| 65982 | ZSCAN18 | Q8TBC5 | XM_011527239.3 | 2729 | XP_011525541.1 | 510 | zinc finger and |
| | | | | | | | SCAN |
| | | | | | | | domain- |
| | | | | | | | containing |
| | | | | | | | protein 18 |
| 65982 | ZSCAN18 | Q8TBC5 | XM_011527238.1 | 2788 | XP_011525540.1 | 510 | zinc finger and |
| | | | | | | | SCAN |
| | | | | | | | domain- |
| | | | | | | | containing |
| | | | | | | | protein 18 |
| 65982 | ZSCAN18 | Q8TBC5 | XM_005259174.5 | 2569 | XP_005259231.1 | 510 | zinc finger and |
| | | | | | | | SCAN |
| | | | | | | | domain- |
| | | | | | | | containing |
| | | | | | | | protein 18 |
| 65982 | ZSCAN18 | Q8TBC5 | NM_001145542.1 | 2605 | NP_001139014.1 | 566 | zinc finger and |
| | | | | | | | SCAN |
| | | | | | | | domain- |
| | | | | | | | containing |
| | | | | | | | protein 18 |
| 65982 | ZSCAN18 | Q8TBC5 | XM_017027170.2 | 2621 | XP_016882659.1 | 509 | zinc finger and |
| | | | | | | | SCAN |
| | | | | | | | domain- |
| | | | | | | | containing |
| | | | | | | | protein 18 |
| 65982 | ZSCAN18 | Q8TBC5 | XM_017027169.2 | 2547 | XP_016882658.1 | 509 | zinc finger and |
| | | | | | | | SCAN |
| | | | | | | | domain- |
| | | | | | | | containing |
| | | | | | | | protein 18 |
| 65982 | ZSCAN18 | Q8TBC5 | XM_017027171.2 | 2559 | XP_016882660.1 | 509 | zinc finger and |
| | | | | | | | SCAN |
| | | | | | | | domain- |
| | | | | | | | containing |
| | | | | | | | protein 18 |
| 65982 | ZSCAN18 | Q8TBC5 | XM_011527237.2 | 2564 | XP_011525539.1 | 565 | zinc finger and |
| | | | | | | | SCAN |
| | | | | | | | domain- |
| | | | | | | | containing |
| | | | | | | | protein 18 |
| 65982 | ZSCAN18 | Q8TBC5 | NM_001145543.2 | 2539 | NP_001139015.1 | 510 | zinc finger and |
| | | | | | | | SCAN |
| | | | | | | | domain- |
| | | | | | | | containing |
| | | | | | | | protein 18 |
| 65982 | ZSCAN18 | Q8TBC5 | NM_001145544.2 | 2073 | NP_001139016.1 | 374 | zinc finger and |
| | | | | | | | SCAN |
| | | | | | | | domain- |
| | | | | | | | containing |
| | | | | | | | protein 18 |
| 65982 | ZSCAN18 | Q8TBC5 | NM_023926.5 | 2783 | NP_076415.3 | 510 | zinc finger and |
| | | | | | | | SCAN |
| | | | | | | | domain- |
| | | | | | | | containing |
| | | | | | | | protein 18 |
| 6925 | TCF4 | P15884 | NM_001243226.3 | 8232 | NP_001230155.2 | 773 | transcription |
| | | | | | | | factor 4 |
| 6925 | TCF4 | P15884 | NM_001369584.1 | 8294 | NP_001356513.1 | 642 | transcription |
| | | | | | | | factor 4 |
| 6925 | TCF4 | P15884 | NM_001369576.1 | 8306 | NP_001356505.1 | 646 | transcription |
| | | | | | | | factor 4 |
| 6925 | TCF4 | P15884 | NM_001369577.1 | 8306 | NP_001356506.1 | 646 | transcription |
| | | | | | | | factor 4 |
| 6925 | TCF4 | P15884 | NM_001369582.1 | 8297 | NP_001356511.1 | 643 | transcription |
| | | | | | | | factor 4 |
| 6925 | TCF4 | P15884 | NM_001243227.2 | 8309 | NP_001230156.1 | 647 | transcription |
| | | | | | | | factor 4 |
| 6925 | TCF4 | P15884 | NM_001369586.1 | 8312 | NP_001356515.1 | 648 | transcription |
| | | | | | | | factor 4 |
| 6925 | TCF4 | P15884 | NM_001369571.1 | 8389 | NP_001356500.1 | 667 | transcription |
| | | | | | | | factor 4 |
| 6925 | TCF4 | P15884 | NM_001369567.1 | 8401 | NP_001356496.1 | 671 | transcription |
| | | | | | | | factor 4 |
| 6925 | TCF4 | P15884 | NM_001243228.2 | 8419 | NP_001230157.1 | 677 | transcription |
| | | | | | | | factor 4 |
| 6925 | TCF4 | P15884 | NM_001243230.2 | 7927 | NP_001230159.1 | 664 | transcription |
| | | | | | | | factor 4 |
| 6925 | TCF4 | P15884 | NM_001369569.1 | 8031 | NP_001356498.1 | 670 | transcription |
| | | | | | | | factor 4 |
| 6925 | TCF4 | P15884 | NM_001369572.1 | 8022 | NP_001356501.1 | 667 | transcription |
| | | | | | | | factor 4 |
| 6925 | TCF4 | P15884 | NM_001369568.1 | 8034 | NP_001356497.1 | 671 | transcription |
| | | | | | | | factor 4 |
| 6925 | TCF4 | P15884 | NM_001369585.1 | 7934 | NP_001356514.1 | 642 | transcription |
| | | | | | | | factor 4 |
| 6925 | TCF4 | P15884 | NM_001369578.1 | 7946 | NP_001356507.1 | 646 | transcription |
| | | | | | | | factor 4 |
| 6925 | TCF4 | P15884 | NM_001369579.1 | 7946 | NP_001356508.1 | 646 | transcription |
| | | | | | | | factor 4 |
| 6925 | TCF4 | P15884 | NM_001369583.1 | 7937 | NP_001356512.1 | 643 | transcription |
| | | | | | | | factor 4 |
| 6925 | TCF4 | P15884 | NM_001369575.1 | 7949 | NP_001356504.1 | 647 | transcription |
| | | | | | | | factor 4 |
| 6925 | TCF4 | P15884 | NM_001369573.1 | 8026 | NP_001356502.1 | 666 | transcription |
| | | | | | | | factor 4 |
| 6925 | TCF4 | P15884 | NM_001369570.1 | 8038 | NP_001356499.1 | 670 | transcription |
| | | | | | | | factor 4 |
| 6925 | TCF4 | P15884 | NM_001369574.1 | 8026 | NP_001356503.1 | 666 | transcription |
| | | | | | | | factor 4 |
| 6925 | TCF4 | P15884 | NM_001330604.3 | 8038 | NP_001317533.1 | 670 | transcription |
| | | | | | | | factor 4 |
| 6925 | TCF4 | P15884 | NM_003199.3 | 8029 | NP_003190.1 | 667 | transcription |
| | | | | | | | factor 4 |
| 6925 | TCF4 | P15884 | NM_001083962.2 | 8041 | NP_001077431.1 | 671 | transcription |
| | | | | | | | factor 4 |
| 6925 | TCF4 | P15884 | NM_001369581.1 | 7941 | NP_001356510.1 | 646 | transcription |
| | | | | | | | factor 4 |
| 6925 | TCF4 | P15884 | NM_001369580.1 | 7941 | NP_001356509.1 | 646 | transcription |
| | | | | | | | factor 4 |
| 6925 | TCF4 | P15884 | NM_001348219.2 | 7932 | NP_001335148.1 | 643 | transcription |
| | | | | | | | factor 4 |
| 6925 | TCF4 | P15884 | NM_001348218.2 | 7944 | NP_001335147.1 | 647 | transcription |
| | | | | | | | factor 4 |
| 6925 | TCF4 | P15884 | NM_001243231.2 | 7903 | NP_001230160.1 | 625 | transcription |
| | | | | | | | factor 4 |
| 6925 | TCF4 | P15884 | NM_001348211.2 | 7915 | NP_001335140.1 | 629 | transcription |
| | | | | | | | factor 4 |
| 6925 | TCF4 | P15884 | NM_001348212.2 | 7653 | NP_001335141.1 | 537 | transcription |
| | | | | | | | factor 4 |
| 6925 | TCF4 | P15884 | NM_001348213.2 | 7668 | NP_001335142.1 | 541 | transcription |
| | | | | | | | factor 4 |
| 6925 | TCF4 | P15884 | NM_001243233.2 | 7668 | NP_001230162.1 | 537 | transcription |
| | | | | | | | factor 4 |
| 6925 | TCF4 | P15884 | NM_001330605.3 | 7606 | NP_001317534.1 | 541 | transcription |
| | | | | | | | factor 4 |
| 6925 | TCF4 | P15884 | NM_001348214.2 | 7474 | NP_001335143.1 | 506 | transcription |
| | | | | | | | factor 4 |
| 6925 | TCF4 | P15884 | NM_001243235.2 | 7477 | NP_001230164.1 | 507 | transcription |
| | | | | | | | factor 4 |
| 6925 | TCF4 | P15884 | NM_001243234.2 | 7489 | NP_001230163.1 | 511 | transcription |
| | | | | | | | factor 4 |
| 6925 | TCF4 | P15884 | NM_001348215.2 | 7422 | NP_001335144.1 | 455 | transcription |
| | | | | | | | factor 4 |
| 6925 | TCF4 | P15884 | NM_001243236.2 | 7550 | NP_001230165.1 | 507 | transcription |
| | | | | | | | factor 4 |
| 6925 | TCF4 | P15884 | NM_001348216.2 | 7562 | NP_001335145.1 | 511 | transcription |
| | | | | | | | factor 4 |
| 6925 | TCF4 | P15884 | NM_001348220.1 | 7942 | NP_001335149.1 | 642 | transcription |
| | | | | | | | factor 4 |
| 6925 | TCF4 | P15884 | NM_001306207.1 | 7945 | NP_001293136.1 | 643 | transcription |
| | | | | | | | factor 4 |
| 6925 | TCF4 | P15884 | NM_001348217.1 | 7957 | NP_001335146.1 | 647 | transcription |
| | | | | | | | factor 4 |
| 6925 | TCF4 | P15884 | NM_001306208.1 | 7776 | NP_001293137.1 | 596 | transcription |
| | | | | | | | factor 4 |
| 6925 | TCF4 | P15884 | NM_001243232.1 | 7788 | NP_001230161.1 | 600 | transcription |
| | | | | | | | factor 4 |
| 6925 | TCF4 | P15884 | XM_017025950.2 | 7063 | XP_016881439.1 | 641 | transcription |
| | | | | | | | factor 4 |
| 6925 | TCF4 | P15884 | XM_006722538.3 | 6794 | XP_006722601.1 | 646 | transcription |
| | | | | | | | factor 4 |
| 6925 | TCF4 | P15884 | XM_017025951.2 | 6650 | XP_016881440.1 | 628 | transcription |
| | | | | | | | factor 4 |
| 6925 | TCF4 | P15884 | XM_024451241.1 | 6470 | XP_024307009.1 | 592 | transcription |
| | | | | | | | factor 4 |
| 6925 | TCF4 | P15884 | XM_017025954.2 | 7025 | XP_016881443.1 | 540 | transcription |
| | | | | | | | factor 4 |
| 6925 | TCF4 | P15884 | XM_005266752.5 | 7015 | XP_005266809.1 | 541 | transcription |
| | | | | | | | factor 4 |
| 6925 | TCF4 | P15884 | XM_005266755.5 | 6472 | XP_005266812.1 | 541 | transcription |
| | | | | | | | factor 4 |
| 6925 | TCF4 | P15884 | XM_017025952.2 | 6664 | XP_016881441.1 | 599 | transcription |
| | | | | | | | factor 4 |
| 6925 | TCF4 | P15884 | XM_017025953.2 | 6432 | XP_016881442.1 | 597 | transcription |
| | | | | | | | factor 4 |
| 6925 | TCF4 | P15884 | XM_005266749.4 | 6444 | XP_005266806.1 | 601 | transcription |
| | | | | | | | factor 4 |
| 6925 | TCF4 | P15884 | XM_017025956.2 | 6426 | XP_016881445.1 | 537 | transcription |
| | | | | | | | factor 4 |
| 6925 | TCF4 | P15884 | XM_005266761.4 | 6802 | XP_005266818.1 | 510 | transcription |
| | | | | | | | factor 4 |
| 6932 | TCF7 | P36402 | XR_948292.1 | 3338 | | | |
| 6932 | TCF7 | P36402 | XR_001742232.1 | 1358 | | | |
| 6932 | TCF7 | P36402 | XR_001742231.1 | 1265 | | | |
| 6932 | TCF7 | P36402 | XR_948294.2 | 1301 | | | |
| 6932 | TCF7 | P36402 | NR_033449.3 | 3095 | | | |
| 6932 | TCF7 | P36402 | NM_001346425.2 | 3381 | NP_001333354.1 | 415 | transcription |
| | | | | | | | factor 7 |
| 6932 | TCF7 | P36402 | NM_003202.5 | 3288 | NP_003193.2 | 384 | transcription |
| | | | | | | | factor 7 |
| 6932 | TCF7 | P36402 | XM_006714682.2 | 3401 | XP_006714745.1 | 396 | transcription |
| | | | | | | | factor 7 |
| 6932 | TCF7 | P36402 | XM_006714684.2 | 3308 | XP_006714747.1 | 365 | transcription |
| | | | | | | | factor 7 |
| 6932 | TCF7 | P36402 | XM_011543606.1 | 1865 | XP_011541908.1 | 402 | transcription |
| | | | | | | | factor 7 |
| 6932 | TCF7 | P36402 | XM_011543604.2 | 1500 | XP_011541906.1 | 416 | transcription |
| | | | | | | | factor 7 |
| 6932 | TCF7 | P36402 | XM_006714678.3 | 2288 | XP_006714741.1 | 460 | transcription |
| | | | | | | | factor 7 |
| 6932 | TCF7 | P36402 | XM_006714679.3 | 2195 | XP_006714742.1 | 429 | transcription |
| | | | | | | | factor 7 |
| 6932 | TCF7 | P36402 | XM_006714685.4 | 1836 | XP_006714748.1 | 345 | transcription |
| | | | | | | | factor 7 |
| 6932 | TCF7 | P36402 | XM_006714686.4 | 1743 | XP_006714749.1 | 314 | transcription |
| | | | | | | | factor 7 |
| 6932 | TCF7 | P36402 | NM_213648.5 | 3042 | NP_998813.1 | 269 | transcription |
| | | | | | | | factor 7 |
| 6932 | TCF7 | P36402 | NM_001346450.2 | 2924 | NP_001333379.1 | 281 | transcription |
| | | | | | | | factor 7 |
| 6932 | TCF7 | P36402 | NM_201634.5 | 2904 | NP_963965.1 | 268 | transcription |
| | | | | | | | factor 7 |
| 6932 | TCF7 | P36402 | NM_001134851.4 | 3291 | NP_001128323.2 | 269 | transcription |
| | | | | | | | factor 7 |
| 6932 | TCF7 | P36402 | NM_201632.5 | 2808 | NP_963963.1 | 269 | transcription |
| | | | | | | | factor 7 |
| 6932 | TCF7 | P36402 | XM_011543613.3 | 1762 | XP_011541915.1 | 227 | transcription |
| | | | | | | | factor 7 |
| 6932 | TCF7 | P36402 | XM_011543608.2 | 3821 | XP_011541910.1 | 320 | transcription |
| | | | | | | | factor 7 |
| 6932 | TCF7 | P36402 | NM_001366502.2 | 2760 | NP_001353431.1 | 244 | transcription |
| | | | | | | | factor 7 |
| 6932 | TCF7 | P36402 | XM_011543609.2 | 1689 | XP_011541911.1 | 289 | transcription |
| | | | | | | | factor 7 |
| 6932 | TCF7 | P36402 | XM_011543607.2 | 7776 | XP_011541909.1 | 341 | transcription |
| | | | | | | | factor 7 |
| 6932 | TCF7 | P36402 | XM_017009790.1 | 3803 | XP_016865279.1 | 213 | transcription |
| | | | | | | | factor 7 |
| 7049 | TGFBR3 | Q03167 | NR_036634.2 | 6436 | | | |
| 7049 | TGFBR3 | Q03167 | NM_001195684.1 | 6308 | NP_001182613.1 | 850 | transforming |
| | | | | | | | growth factor |
| | | | | | | | beta receptor |
| | | | | | | | type 3 |
| 7049 | TGFBR3 | Q03167 | NM_001195683.2 | 6336 | NP_001182612.1 | 850 | transforming |
| | | | | | | | growth factor |
| | | | | | | | beta receptor |
| | | | | | | | type 3 |
| 7049 | TGFBR3 | Q03167 | NM_003243.5 | 6339 | NP_003234.2 | 851 | transforming |
| | | | | | | | growth factor |
| | | | | | | | beta receptor |
| | | | | | | | type 3 |
| 7049 | TGFBR3 | Q03167 | XM_006710867.2 | 3813 | XP_006710930.1 | 851 | transforming |
| | | | | | | | growth factor |
| | | | | | | | beta receptor |
| | | | | | | | type 3 |
| 7710 | ZNF154 | Q13106 | NR_110975.2 | 3669 | | | |
| 7710 | ZNF154 | Q13106 | NR_110974.2 | 6815 | | | |
| 7710 | ZNF154 | Q13106 | XR_001753757.1 | 2736 | | | |
| 7710 | ZNF154 | Q13106 | NM_001085384.3 | 6907 | NP_001078853.1 | 437 | |
| 78991 | PCYOX1L | Q8NBM8 | NM_001301054.2 | 2523 | NP_001287983.1 | 477 | prenylcysteine |
| | | | | | | | oxidase-like |
| 78991 | PCYOX1L | Q8NBM8 | NM_001301057.2 | 2346 | NP_001287986.1 | 418 | prenylcysteine |
| | | | | | | | oxidase-like |
| 78991 | PCYOX1L | Q8NBM8 | NM_024028.4 | 2507 | NP_076933.3 | 494 | prenylcysteine |
| | | | | | | | oxidase-like |
| 79156 | PLEKHF1 | Q96S99 | XM_005259256.3 | 2028 | XP_005259313.1 | 364 | pleckstrin |
| | | | | | | | homology |
| | | | | | | | domain- |
| | | | | | | | containing |
| | | | | | | | family F |
| | | | | | | | member 1 |
| 79156 | PLEKHF1 | Q96S99 | XM_011527309.3 | 3384 | XP_011525611.1 | 279 | pleckstrin |
| | | | | | | | homology |
| | | | | | | | domain- |
| | | | | | | | containing |
| | | | | | | | family F |
| | | | | | | | member 1 |
| 79156 | PLEKHF1 | Q96S99 | NM_024310.5 | 1699 | NP_077286.3 | 279 | |
| 79368 | FCRL2 | Q96LA5 | NR_125358.2 | 1671 | | | |
| 79368 | FCRL2 | Q96LA5 | XR_001737404.1 | 1485 | | | |
| 79368 | FCRL2 | Q96LA5 | XR_001737403.1 | 1507 | | | |
| 79368 | FCRL2 | Q96LA5 | XM_017002318.1 | 2618 | XP_016857807.1 | 430 | Fc receptor- |
| | | | | | | | like protein 2 |
| 79368 | FCRL2 | Q96LA5 | XM_017002317.1 | 2698 | XP_016857806.1 | 457 | Fc receptor- |
| | | | | | | | like protein 2 |
| 79368 | FCRL2 | Q96LA5 | XM_011509976.2 | 2859 | XP_011508278.1 | 486 | Fc receptor- |
| | | | | | | | like protein 2 |
| 79368 | FCRL2 | Q96LA5 | XM_017002319.1 | 2610 | XP_016857808.1 | 428 | Fc receptor- |
| | | | | | | | like protein 2 |
| 79368 | FCRL2 | Q96LA5 | XM_017002316.1 | 2895 | XP_016857805.1 | 523 | Fc receptor- |
| | | | | | | | like protein 2 |
| 79368 | FCRL2 | Q96LA5 | NM_001159488.2 | 2408 | NP_001152960.1 | 444 | Fc receptor- |
| | | | | | | | like protein 2 |
| 79368 | FCRL2 | Q96LA5 | NM_030764.4 | 2589 | NP_110391.2 | 508 | Fc receptor- |
| | | | | | | | like protein 2 |
| 79368 | FCRL2 | Q96LA5 | XM_011509975.3 | 1966 | XP_011508277.1 | 491 | Fc receptor- |
| | | | | | | | like protein 2 |
| 79368 | FCRL2 | Q96LA5 | XM_011509974.3 | 2030 | XP_011508276.1 | 513 | Fc receptor- |
| | | | | | | | like protein 2 |
| 79368 | FCRL2 | Q96LA5 | XM_006711535.3 | 1371 | XP_006711598.1 | 313 | Fc receptor- |
| | | | | | | | like protein 2 |
| 79600 | TCTN1 | Q2MV58 | NR_135088.2 | 2336 | | | |
| 79600 | TCTN1 | Q2MV58 | XR_944717.3 | 1921 | | | |
| 79600 | TCTN1 | Q2MV58 | XR_429116.3 | 1695 | | | |
| 79600 | TCTN1 | Q2MV58 | XR_243021.4 | 1680 | | | |
| 79600 | TCTN1 | Q2MV58 | XR_243022.4 | 1554 | | | |
| 79600 | TCTN1 | Q2MV58 | XM_006719600.3 | 1832 | XP_006719663.1 | 414 | tectonic-1 |
| 79600 | TCTN1 | Q2MV58 | XM_006719596.3 | 2118 | XP_006719659.1 | 414 | tectonic-1 |
| 79600 | TCTN1 | Q2MV58 | XM_006719598.3 | 2088 | XP_006719661.1 | 414 | tectonic-1 |
| 79600 | TCTN1 | Q2MV58 | XM_011538734.3 | 1884 | XP_011537036.1 | 572 | tectonic-1 |
| 79600 | TCTN1 | Q2MV58 | XM_006719595.3 | 2095 | XP_006719658.1 | 414 | tectonic-1 |
| 79600 | TCTN1 | Q2MV58 | XM_006719597.4 | 1816 | XP_006719660.1 | 414 | tectonic-1 |
| 79600 | TCTN1 | Q2MV58 | XM_006719594.3 | 1989 | XP_006719657.1 | 536 | tectonic-1 |
| 79600 | TCTN1 | Q2MV58 | XM_017019964.1 | 1827 | XP_016875453.1 | 482 | tectonic-1 |
| 79600 | TCTN1 | Q2MV58 | XM_017019969.2 | 1851 | XP_016875458.1 | 400 | tectonic-1 |
| 79600 | TCTN1 | Q2MV58 | XM_006719599.3 | 1893 | XP_006719662.1 | 414 | tectonic-1 |
| 79600 | TCTN1 | Q2MV58 | NM_001173975.3 | 2268 | NP_001167446.1 | 531 | tectonic-1 |
| 79600 | TCTN1 | Q2MV58 | NM_001173976.2 | 2151 | NP_001167447.1 | 483 | tectonic-1 |
| 79600 | TCTN1 | Q2MV58 | NM_001319681.2 | 2380 | NP_001306610.1 | 409 | tectonic-1 |
| 79600 | TCTN1 | Q2MV58 | NM_024549.6 | 2165 | NP_078825.2 | 573 | tectonic-1 |
| 79600 | TCTN1 | Q2MV58 | NM_001082538.3 | 2222 | NP_001076007.1 | 592 | tectonic-1 |
| 79600 | TCTN1 | Q2MV58 | NM_001082537.3 | 2207 | NP_001076006.1 | 587 | tectonic-1 |
| 79600 | TCTN1 | Q2MV58 | NM_001319680.2 | 2060 | NP_001306609.1 | 538 | tectonic-1 |
| 79600 | TCTN1 | Q2MV58 | XM_011538733.3 | 1880 | XP_011537035.1 | 578 | tectonic-1 |
| 79600 | TCTN1 | Q2MV58 | XM_011538735.2 | 1775 | XP_011537037.1 | 543 | tectonic-1 |
| 79600 | TCTN1 | Q2MV58 | XM_011538737.3 | 1694 | XP_011537039.1 | 516 | tectonic-1 |
| 79600 | TCTN1 | Q2MV58 | XM_011538738.3 | 1568 | XP_011537040.1 | 474 | tectonic-1 |
| 79600 | TCTN1 | Q2MV58 | XM_005253934.4 | 1406 | XP_005253991.1 | 420 | tectonic-1 |
| 79600 | TCTN1 | Q2MV58 | XM_005253935.4 | 1391 | XP_005253992.1 | 415 | tectonic-1 |
| 79600 | TCTN1 | Q2MV58 | NM_001319682.3 | 2078 | NP_001306611.1 | 189 | tectonic-1 |
| 79600 | TCTN1 | Q2MV58 | XM_017019966.2 | 2254 | XP_016875455.1 | 414 | tectonic-1 |
| 79600 | TCTN1 | Q2MV58 | XM_017019968.2 | 1789 | XP_016875457.1 | 414 | tectonic-1 |
| 79690 | GAL3ST4 | Q96RP7 | NM_024637.5 | 2405 | NP_078913.3 | 486 | |
| 79817 | MOB3B | Q86TA1 | NM_024761.55 | 6487 | NP_079037.3 | 216 | |
| 79891 | ZNF671 | Q8TAW3 | XM_017027314.1 | 2931 | XP_016882803.1 | 457 | zinc finger |
| | | | | | | | protein 671 |
| 79891 | ZNF671 | Q8TAW3 | NM_001321375.2 | 2304 | NP_001308304.1 | 436 | zinc finger |
| | | | | | | | protein 671 |
| 79891 | ZNF671 | Q8TAW3 | NM_024833.3 | 2431 | NP_079109.2 | 534 | zinc finger |
| | | | | | | | protein 671 |
| 79891 | ZNF671 | Q8TAW3 | NM_001321376.2 | 2469 | NP_001308305.1 | 457 | zinc finger |
| | | | | | | | protein 671 |
| 79899 | PRR5L | Q6MZQ0 | NM_001160167.2 | 3851 | NP_001153639.1 | 368 | proline-rich |
| | | | | | | | protein 5-like |
| 79899 | PRR5L | Q6MZQ0 | NM_024841.5 | 3950 | NP_079117.3 | 368 | proline-rich |
| | | | | | | | protein 5-like |
| 79899 | PRR5L | Q6MZQ0 | NM_001160168.2 | 3363 | NP_001153640.1 | 240 | proline-rich |
| | | | | | | | protein 5-like |
| 79899 | PRR5L | Q6MZQ0 | NM_001160169.1 | 3588 | NP_001153641.1 | 205 | proline-rich |
| | | | | | | | protein 5-like |
| 802377 | ELL3 | Q9HB65 | NM_025165.3 | 1755 | NP_079441.1 | 397 | |
| 8111 | GPR68 | Q15743 | XM_005268112.3 | 2720 | XP_005268169.1 | 375 | ovarian cancer |
| | | | | | | | G-protein |
| | | | | | | | coupled |
| | | | | | | | receptor 1 |
| 8111 | GPR68 | Q15743 | XM_005268111.3 | 2780 | XP_005268168.1 | 375 | ovarian cancer |
| | | | | | | | G-protein |
| | | | | | | | coupled |
| | | | | | | | receptor 1 |
| 8111 | GPR68 | Q15743 | NM_003485.3 | 2834 | NP_003476.3 | 365 | |
| 8111 | GPR68 | Q15743 | NM_001348437.1 | 3021 | NP_001335366.1 | 365 | |
| 8111 | GPR68 | Q15743 | XM_005268110.4 | 4000 | XP_005268167.1 | 375 | ovarian cancer |
| | | | | | | | G-protein |
| | | | | | | | coupled |
| | | | | | | | receptor 1 |
| 8111 | GPR68 | Q15743 | NM_001177676.2 | 3056 | NP_001171147.1 | 365 | |
| 8111 | GPR68 | Q15743 | XM_011537196.2 | 2864 | XP_011535498.1 | 375 | ovarian cancer |
| | | | | | | | G-protein |
| | | | | | | | coupled |
| | | | | | | | receptor 1 |
| 8111 | GPR68 | Q15743 | XM_011537197.3 | 3143 | XP_011535499.1 | 375 | ovarian cancer |
| | | | | | | | G-protein |
| | | | | | | | coupled |
| | | | | | | | receptor 1 |
| 8111 | GPR68 | Q15743 | XM_01153198.27 | 3370 | XP_011535500.1 | 375 | ovarian cancer |
| | | | | | | | G-protein |
| | | | | | | | coupled |
| | | | | | | | receptor 1 |
| 8111 | GPR68 | Q15743 | XM_006720262.3 | 2746 | XP_006720325.1 | 375 | ovarian cancer |
| | | | | | | | G-protein |
| | | | | | | | coupled |
| | | | | | | | receptor 1 |
| 8111 | GPR68 | Q15743 | XM_011537199.2 | 2815 | XP_011535501.1 | 375 | ovarian cancer |
| | | | | | | | G-protein |
| | | | | | | | coupled |
| | | | | | | | receptor 1 |
| 8115 | TCL1A | P56279 | NR_049726.2 | 1569 | | | |
| 8115 | TCL1A | P56279 | XM_017021676.2 | 1001 | XP_016877165.1 | 114 | T-cell |
| | | | | | | | leukemia/ |
| | | | | | | | lymphoma |
| | | | | | | | protein 1A |
| 8115 | TCL1A | P56279 | XM_017021677.2 | 1006 | XP_016877166.1 | 114 | T-cell |
| | | | | | | | leukemia/ |
| | | | | | | | lymphoma |
| | | | | | | | protein 1A |
| 8115 | TCL1A | P56279 | NM_001098725.2 | 1340 | NP_001092195.1 | 114 | |
| 8115 | TCL1A | P56279 | NM_021966.3 | 1345 | NP_068801.1 | 114 | |
| 81563 | Clorf21 | Q9H246 | NM_030806.4 | 10293 | NP_110433.1 | 121 | |
| 81571 | MIR600HG | | NR_026677.1 | 6002 | | | |
| 83667 | SESN2 | P58004 | XR_946773.1 | 3377 | | | |
| 83667 | SESN2 | P58004 | NM_031459.5 | 3462 | NP_113647.1 | 480 | sestrin-2 |
| 83888 | FGFBP2 | Q9BYJ0 | NM_031950.4 | 1105 | NP_114156.1 | 223 | |
| 8409 | UXT | Q9UBK9 | NR_045560.2 | 660 | | | |
| 8409 | UXT | Q9UBK9 | NR_045559.2 | 705 | | | |
| 8409 | UXT | Q9UBK9 | NM_004182.4 | 595 | NP_004173.1 | 157 | protein UXT |
| 8409 | UXT | Q9UBK9 | NM_153477.3 | 773 | NP_705582.1 | 169 | protein UXT |
| 84329 | HVCN1 | Q96D96 | NM_001256413.2 | 1661 | NP_001243342.1 | 253 | voltage-gated |
| | | | | | | | hydrogen |
| | | | | | | | channel 1 |
| 84329 | HVCN1 | Q96D96 | NM_001040107.2 | 1701 | NP_001035196.1 | 273 | voltage-gated |
| | | | | | | | hydrogen |
| | | | | | | | channel 1 |
| 84329 | HVCN1 | Q96D96 | NM_032369.4 | 1685 | NP_115745.2 | 273 | voltage-gated |
| | | | | | | | hydrogen |
| | | | | | | | channel 1 |
| 84329 | HVCN1 | Q96D96 | XM_011538846.2 | 1752 | XP_011537148.1 | 273 | voltage-gated |
| | | | | | | | hydrogen |
| | | | | | | | channel 1 |
| 84329 | HVCN1 | Q96D96 | XM_017020026.2 | 1766 | XP_016875515.1 | 320 | voltage-gated |
| | | | | | | | hydrogen |
| | | | | | | | channel 1 |
| 84329 | HVCN1 | Q96D96 | XM_011538847.1 | 1653 | XP_011537149.1 | 273 | voltage-gated |
| | | | | | | | hydrogen |
| | | | | | | | channel 1 |
| 84329 | HVCN1 | Q96D96 | XM_024449225.1 | 1668 | XP_024304993.1 | 273 | voltage-gated |
| | | | | | | | hydrogen |
| | | | | | | | channel 1 |
| 84329 | HVCN1 | Q96D96 | XM_011538842.2 | 1783 | XP_011537144.1 | 320 | voltage-gated |
| | | | | | | | hydrogen |
| | | | | | | | channel 1 |
| 84329 | HVCN1 | Q96D96 | XM_011538839.2 | 1793 | XP_011537141.1 | 320 | voltage-gated |
| | | | | | | | hydrogen |
| | | | | | | | channel 1 |
| 84329 | HVCN1 | Q96D96 | XM_005253948.2 | 1721 | XP_005254005.1 | 273 | voltage-gated |
| | | | | | | | hydrogen |
| | | | | | | | channel 1 |
| 84329 | HVCN1 | Q96D96 | XM_011538840.3 | 1826 | XP_011537142.1 | 320 | voltage-gated |
| | | | | | | | hydrogen |
| | | | | | | | channel 1 |
| 84329 | HVCN1 | Q96D96 | XM_011538844.2 | 1798 | XP_011537146.1 | 320 | voltage-gated |
| | | | | | | | hydrogen |
| | | | | | | | channel 1 |
| 84329 | HVCN1 | Q96D96 | XM_011538841.2 | 1803 | XP_011537143.1 | 320 | voltage-gated |
| | | | | | | | hydrogen |
| | | | | | | | channel 1 |
| 84329 | HVCN1 | Q96D96 | XM_017020027.1 | 1652 | XP_016875516.1 | 273 | voltage-gated |
| | | | | | | | hydrogen |
| | | | | | | | channel 1 |
| 84329 | HVCN1 | Q96D96 | XM_011538845.2 | 1658 | XP_011537147.1 | 273 | voltage-gated |
| | | | | | | | hydrogen |
| | | | | | | | channel 1 |
| 84329 | HVCN1 | Q96D96 | XM_011538838.2 | 1770 | XP_011537140.1 | 320 | voltage-gated |
| | | | | | | | hydrogen |
| | | | | | | | channel 1 |
| 8511 | MMP23A | | NR_002946.1 | 906 | | | |
| 8530 | CST7 | 076096 | NM_003650.4 | 891 | NP_003641.3 | 145 | |
| 85315 | PAQR8 | Q8TEZ7 | NM_133367.5 | 4715 | NP_588608.1 | 354 | |
| 8718 | TNFRSF25 | Q93038 | NM_148970.2 | 1419 | NP_683871.1 | 234 | tumor necrosis |
| | | | | | | | factor receptor |
| | | | | | | | superfamily |
| | | | | | | | member 25 |
| 8718 | TNFRSF25 | Q93038 | NM_148967.2 | 1833 | NP_683868.1 | 372 | tumor necrosis |
| | | | | | | | factor receptor |
| | | | | | | | superfamily |
| | | | | | | | member 25 |
| 8718 | TNFRSF25 | Q93038 | NM_148966.2 | 1857 | NP_683867.1 | 380 | tumor necrosis |
| | | | | | | | factor receptor |
| | | | | | | | superfamily |
| | | | | | | | member 25 |
| 8718 | TNFRSF25 | Q93038 | NM_003790.3 | 1968 | NP_003781.1 | 417 | tumor necrosis |
| | | | | | | | factor receptor |
| | | | | | | | superfamily |
| | | | | | | | member 25 |
| 8718 | TNFRSF25 | Q93038 | NM_148965.2 | 1995 | NP_683866.1 | 426 | tumor necrosis |
| | | | | | | | factor receptor |
| | | | | | | | superfamily |
| | | | | | | | member 25 |
| 8718 | TNFRSF25 | Q93038 | NM_001039664.2 | 872 | NP_001034753.1 | 181 | tumor necrosis |
| | | | | | | | factor receptor |
| | | | | | | | superfamily |
| | | | | | | | member 25 |
| 8819 | SAP30 | O75446 | NM_003864.4 | 1097 | NP_003855.1 | 220 | |
| 8857 | FCGBP | Q9Y6R7 | NM_003890.2 | 16407 | NP_003881.2 | 5405 | |
| 9057 | SLC7A6 | Q92536 | XR_243433.3 | 5251 | | | |
| 9057 | SLC7A6 | Q92536 | XR_001752018.1 | 5304 | | | |
| 9057 | SLC7A6 | Q92536 | XR_002957851.1 | 5450 | | | |
| 9057 | SLC7A6 | Q92536 | XM_011523438.2 | 6142 | XP_011521740.1 | 515 | Y + L amino |
| | | | | | | | acid |
| | | | | | | | transporter 2 |
| 9057 | SLC7A6 | Q92536 | NM_001076785.3 | 6342 | NP_001070253.1 | 515 | |
| 9057 | SLC7A6 | Q92536 | NM_003983.6 | 6255 | NP_003974.3 | 515 | |
| 9057 | SLC7A6 | Q92536 | XM_024450486.1 | 6464 | XP_024306254.1 | 515 | Y + L amino |
| | | | | | | | acid |
| | | | | | | | transporter 2 |
| 9057 | SLC7A6 | Q92536 | XM_011523433.1 | 6367 | XP_011521735.1 | 515 | Y + L amino |
| | | | | | | | acid |
| | | | | | | | transporter 2 |
| 9057 | SLC7A6 | Q92536 | XM_011523434.1 | 6432 | XP_011521736.1 | 515 | Y + L amino |
| | | | | | | | acid |
| | | | | | | | transporter 2 |
| 9057 | SLC7A6 | Q92536 | XM_024450487.1 | 6314 | XP_024306255.1 | 515 | Y + L amino |
| | | | | | | | acid |
| | | | | | | | transporter 2 |
| 9057 | SLC7A6 | Q92536 | XM_024450488.1 | 6287 | XP_024306256.1 | 515 | Y + L amino |
| | | | | | | | acid |
| | | | | | | | transporter 2 |
| 91523 | PCED1B | Q96HM7 | NM_138371.3 | 2310 | NP_612380.1 | 432 | |
| 91523 | PCED1B | Q96HM7 | NM_001281429.2 | 1842 | NP_001268358.1 | 432 | |
| 91523 | PCED1B | Q96HM7 | XM_005269224.5 | 2030 | XP_005269281.1 | 432 | PC-esterase |
| | | | | | | | domain- |
| | | | | | | | containing |
| | | | | | | | protein 1B |
| 91523 | PCED1B | Q96HM7 | XM_017020208.1 | 2327 | XP_016875697.1 | 432 | PC-esterase |
| | | | | | | | domain- |
| | | | | | | | containing |
| | | | | | | | protein 1B |
| 91523 | PCED1B | Q96HM7 | XM_017020214.1 | 2511 | XP_016875703.1 | 432 | PC-esterase |
| | | | | | | | domain- |
| | | | | | | | containing |
| | | | | | | | protein 1B |
| 91523 | PCED1B | Q96HM7 | XM_017020213.1 | 2395 | XP_016875702.1 | 432 | PC-esterase |
| | | | | | | | domain- |
| | | | | | | | containing |
| | | | | | | | protein 1B |
| 91523 | PCED1B | Q96HM7 | XM_017020215.1 | 2366 | XP_016875704.1 | 432 | PC-esterase |
| | | | | | | | domain- |
| | | | | | | | containing |
| | | | | | | | protein 1B |
| 91523 | PCED1B | Q96HM7 | XM_017020207.1 | 2250 | XP_016875696.1 | 432 | PC-esterase |
| | | | | | | | domain- |
| | | | | | | | containing |
| | | | | | | | protein 1B |
| 91523 | PCED1B | Q96HM7 | XM_011538978.2 | 2193 | XP_011537280.1 | 432 | PC-esterase |
| | | | | | | | domain- |
| | | | | | | | containing |
| | | | | | | | protein 1B |
| 91523 | PCED1B | Q96HM7 | XM_017020209.1 | 2082 | XP_016875698.1 | 432 | PC-esterase |
| | | | | | | | domain- |
| | | | | | | | containing |
| | | | | | | | protein 1B |
| 91523 | PCED1B | Q96HM7 | XM_017020210.1 | 3999 | XP_016875699.1 | 432 | PC-esterase |
| | | | | | | | domain- |
| | | | | | | | containing |
| | | | | | | | protein 1B |
| 91523 | PCED1B | Q96HM7 | XM_017020216.1 | 3570 | XP_016875705.1 | 432 | PC-esterase |
| | | | | | | | domain- |
| | | | | | | | containing |
| | | | | | | | protein 1B |
| 91523 | PCED1B | Q96HM7 | XM_017020211.1 | 2279 | XP_016875700.1 | 432 | PC-esterase |
| | | | | | | | domain- |
| | | | | | | | containing |
| | | | | | | | protein 1B |
| 91523 | PCED1B | Q96HM7 | XM_017020212.1 | 2022 | XP_016875701.1 | 432 | PC-esterase |
| | | | | | | | domain- |
| | | | | | | | containing |
| | | | | | | | protein 1B |
| 91782 | CHMP7 | Q8WUX9 | NM_152272.5 | 3410 | NP_689485.1 | 453 | charged |
| | | | | | | | multivesicular |
| | | | | | | | body protein 7 |
| 91782 | CHMP7 | Q8WUX9 | XM_017013964.1 | 4560 | XP_016869453.1 | 343 | charged |
| | | | | | | | multivesicular |
| | | | | | | | body protein 7 |
| 91782 | CHMP7 | Q8WUX9 | XM_017013962.1 | 3762 | XP_016869451.1 | 385 | charged |
| | | | | | | | multivesicular |
| | | | | | | | body protein 7 |
| 91782 | CHMP7 | Q8WUX9 | XM_024447327.1 | 3381 | XP_024303095.1 | 385 | charged |
| | | | | | | | multivesicular |
| | | | | | | | body protein 7 |
| 91782 | CHMP7 | Q8WUX9 | XM_024447328.1 | 3167 | XP_024303096.1 | 343 | charged |
| | | | | | | | multivesicular |
| | | | | | | | body protein 7 |
| 91782 | CHMP7 | Q8WUX9 | XM_024447329.1 | 3106 | XP_024303097.1 | 319 | charged |
| | | | | | | | multivesicular |
| | | | | | | | body protein 7 |
| 91782 | CHMP7 | Q8WUX9 | XM_017013961.2 | 3062 | XP_016869450.1 | 495 | charged |
| | | | | | | | multivesicular |
| | | | | | | | body protein 7 |
| 91782 | CHMP7 | Q8WUX9 | NM_001363183.2 | 2756 | NP_001350112.1 | 429 | charged |
| | | | | | | | multivesicular |
| | | | | | | | body protein 7 |
| 91782 | CHMP7 | Q8WUX9 | NM_001317899.2 | 2647 | NP_001304828.1 | 343 | charged |
| | | | | | | | multivesicular |
| | | | | | | | body protein 7 |
| 9214 | FCMR | O60667 | XR_921999.3 | 1965 | | | |
| 9214 | FCMR | O60667 | XM_005273351.4 | 2998 | XP_005273408.1 | 330 | fas apoptotic |
| | | | | | | | inhibitory |
| | | | | | | | molecule 3 |
| 9214 | FCMR | O60667 | NM_001142473.2 | 2626 | NP_001135945.1 | 278 | fas apoptotic |
| | | | | | | | inhibitory |
| | | | | | | | molecule 3 |
| 9214 | FCMR | O60667 | NM_001193338.2 | 2831 | NP_001180267.1 | 306 | fas apoptotic |
| | | | | | | | inhibitory |
| | | | | | | | molecule 3 |
| 9214 | FCMR | O60667 | NM_005449.5 | 2962 | NP_005440.1 | 390 | fas apoptotic |
| | | | | | | | inhibitory |
| | | | | | | | molecule 3 |
| 9214 | FCMR | O60667 | XM_005273352.5 | 1878 | XP_005273409.1 | 299 | fas apoptotic |
| | | | | | | | inhibitory |
| | | | | | | | molecule 3 |
| 9241 | NOG | Q13253 | NM_005450.6 | 1913 | NP_005441.1 | 232 | |
| 9289 | ADGRG1 | Q9Y653 | XM_006721340.3 | 4047 | XP_006721403.1 | 698 | adhesion G- |
| | | | | | | | protein |
| | | | | | | | coupled |
| | | | | | | | receptor G1 |
| 9289 | ADGRG1 | Q9Y653 | XM_011523465.2 | 3873 | XP_011521767.1 | 693 | adhesion G- |
| | | | | | | | protein |
| | | | | | | | coupled |
| | | | | | | | receptor G1 |
| 9289 | ADGRG1 | Q9Y653 | XM_005256242.4 | 3743 | XP_005256299.1 | 698 | adhesion G- |
| | | | | | | | protein |
| | | | | | | | coupled |
| | | | | | | | receptor G1 |
| 9289 | ADGRG1 | Q9Y653 | NM_001370440.1 | 4350 | NP_001357369.1 | 687 | adhesion G- |
| | | | | | | | protein |
| | | | | | | | coupled |
| | | | | | | | receptor G1 |
| 9289 | ADGRG1 | Q9Y653 | NM_001370428.1 | 4364 | NP_001357357.1 | 693 | adhesion G- |
| | | | | | | | protein |
| | | | | | | | coupled |
| | | | | | | | receptor G1 |
| 9289 | ADGRG1 | Q9Y653 | NM_001370436.1 | 4346 | NP_001357365.1 | 687 | adhesion G- |
| | | | | | | | protein |
| | | | | | | | coupled |
| | | | | | | | receptor G1 |
| 9289 | ADGRG1 | Q9Y653 | NM_001290142.2 | 3854 | NP_001277071.1 | 523 | adhesion G- |
| | | | | | | | protein |
| | | | | | | | coupled |
| | | | | | | | receptor G1 |
| 9289 | ADGRG1 | Q9Y653 | NM_001370451.1 | 4303 | NP_001357380.1 | 512 | adhesion G- |
| | | | | | | | protein |
| | | | | | | | coupled |
| | | | | | | | receptor G1 |
| 9289 | ADGRG1 | Q9Y653 | NM_001370430.1 | 4246 | NP_001357359.1 | 693 | adhesion G- |
| | | | | | | | protein |
| | | | | | | | coupled |
| | | | | | | | receptor G1 |
| 9289 | ADGRG1 | Q9Y653 | NM_001370438.1 | 4228 | NP_001357367.1 | 687 | adhesion G- |
| | | | | | | | protein |
| | | | | | | | coupled |
| | | | | | | | receptor G1 |
| 9289 | ADGRG1 | Q9Y653 | NM_001290143.2 | 4203 | NP_001277072.1 | 518 | adhesion G- |
| | | | | | | | protein |
| | | | | | | | coupled |
| | | | | | | | receptor G1 |
| 9289 | ADGRG1 | Q9Y653 | NM_001370435.1 | 4208 | NP_001357364.1 | 687 | adhesion G- |
| | | | | | | | protein |
| | | | | | | | coupled |
| | | | | | | | receptor G1 |
| 9289 | ADGRG1 | Q9Y653 | XM_005256240.4 | 3682 | XP_005256297.1 | 698 | adhesion G- |
| | | | | | | | protein |
| | | | | | | | coupled |
| | | | | | | | receptor G1 |
| 9289 | ADGRG1 | Q9Y653 | NM_001370431.1 | 4315 | NP_001357360.1 | 693 | adhesion G- |
| | | | | | | | protein |
| | | | | | | | coupled |
| | | | | | | | receptor G1 |
| 9289 | ADGRG1 | Q9Y653 | NM_001370437.1 | 4297 | NP_001357366.1 | 687 | adhesion G- |
| | | | | | | | protein |
| | | | | | | | coupled |
| | | | | | | | receptor G1 |
| 9289 | ADGRG1 | Q9Y653 | NM_001370453.1 | 4334 | NP_001357382.1 | 512 | adhesion G- |
| | | | | | | | protein |
| | | | | | | | coupled |
| | | | | | | | receptor G1 |
| 9289 | ADGRG1 | Q9Y653 | NM_005682.7 | 4311 | NP_005673.3 | 693 | adhesion G- |
| | | | | | | | protein |
| | | | | | | | coupled |
| | | | | | | | receptor G1 |
| 9289 | ADGRG1 | Q9Y653 | NM_001145770.3 | 4293 | NP_001139242.1 | 687 | adhesion G- |
| | | | | | | | protein |
| | | | | | | | coupled |
| | | | | | | | receptor G1 |
| 9289 | ADGRG1 | Q9Y653 | NM_001370434.1 | 4308 | NP_001357363.1 | 692 | adhesion G- |
| | | | | | | | protein |
| | | | | | | | coupled |
| | | | | | | | receptor G1 |
| 9289 | ADGRG1 | Q9Y653 | NM_001370442.1 | 4137 | NP_001357371.1 | 635 | adhesion G- |
| | | | | | | | protein |
| | | | | | | | coupled |
| | | | | | | | receptor G1 |
| 9289 | ADGRG1 | Q9Y653 | NM_001370433.1 | 4190 | NP_001357362.1 | 692 | adhesion G- |
| | | | | | | | protein |
| | | | | | | | coupled |
| | | | | | | | receptor G1 |
| 9289 | ADGRG1 | Q9Y653 | NM_001370432.1 | 4193 | NP_001357361.1 | 693 | adhesion G- |
| | | | | | | | protein |
| | | | | | | | coupled |
| | | | | | | | receptor G1 |
| 9289 | ADGRG1 | Q9Y653 | NM_001370439.1 | 4175 | NP_001357368.1 | 687 | adhesion G- |
| | | | | | | | protein |
| | | | | | | | coupled |
| | | | | | | | receptor G1 |
| 9289 | ADGRG1 | Q9Y653 | NM_001370441.1 | 4172 | NP_001357370.1 | 686 | adhesion G- |
| | | | | | | | protein |
| | | | | | | | coupled |
| | | | | | | | receptor G1 |
| 9289 | ADGRG1 | Q9Y653 | XM_006721339.4 | 3790 | XP_006721402.1 | 698 | adhesion G- |
| | | | | | | | protein |
| | | | | | | | coupled |
| | | | | | | | receptor G1 |
| 9289 | ADGRG1 | Q9Y653 | XM_006721345.3 | 3758 | XP_006721408.1 | 693 | adhesion G- |
| | | | | | | | protein |
| | | | | | | | coupled |
| | | | | | | | receptor G1 |
| 9289 | ADGRG1 | Q9Y653 | XM_005256238.2 | 4199 | XP_005256295.1 | 698 | adhesion G- |
| | | | | | | | protein |
| | | | | | | | coupled |
| | | | | | | | receptor G1 |
| 9289 | ADGRG1 | Q9Y653 | XM_006721347.2 | 4181 | XP_006721410.1 | 692 | adhesion G- |
| | | | | | | | protein |
| | | | | | | | coupled |
| | | | | | | | receptor G1 |
| 9289 | ADGRG1 | Q9Y653 | XM_005256255.2 | 4166 | XP_005256312.1 | 687 | adhesion G- |
| | | | | | | | protein |
| | | | | | | | coupled |
| | | | | | | | receptor G1 |
| 9289 | ADGRG1 | Q9Y653 | XM_006721341.2 | 4004 | XP_006721404.1 | 698 | adhesion G- |
| | | | | | | | protein |
| | | | | | | | coupled |
| | | | | | | | receptor G1 |
| 9289 | ADGRG1 | Q9Y653 | XM_006721344.2 | 3989 | XP_006721407.1 | 693 | adhesion G- |
| | | | | | | | protein |
| | | | | | | | coupled |
| | | | | | | | receptor G1 |
| 9289 | ADGRG1 | Q9Y653 | XM_005256245.2 | 3886 | XP_005256302.1 | 698 | adhesion G- |
| | | | | | | | protein |
| | | | | | | | coupled |
| | | | | | | | receptor G1 |
| 9289 | ADGRG1 | Q9Y653 | XM_005256254.2 | 3871 | XP_005256311.1 | 693 | adhesion G- |
| | | | | | | | protein |
| | | | | | | | coupled |
| | | | | | | | receptor G1 |
| 9289 | ADGRG1 | Q9Y653 | XM_017023892.1 | 3853 | XP_016879381.1 | 687 | adhesion G- |
| | | | | | | | protein |
| | | | | | | | coupled |
| | | | | | | | receptor G1 |
| 9289 | ADGRG1 | Q9Y653 | XM_006721338.2 | 3982 | XP_006721401.1 | 698 | adhesion G- |
| | | | | | | | protein |
| | | | | | | | coupled |
| | | | | | | | receptor G1 |
| 9289 | ADGRG1 | Q9Y653 | XM_006721342.2 | 3967 | XP_006721405.1 | 693 | adhesion G- |
| | | | | | | | protein |
| | | | | | | | coupled |
| | | | | | | | receptor G1 |
| 9289 | ADGRG1 | Q9Y653 | XM_005256237.2 | 3860 | XP_005256294.1 | 698 | adhesion G- |
| | | | | | | | protein |
| | | | | | | | coupled |
| | | | | | | | receptor G1 |
| 9289 | ADGRG1 | Q9Y653 | NM_001145772.3 | 4401 | NP_001139244.1 | 687 | adhesion G- |
| | | | | | | | protein |
| | | | | | | | coupled |
| | | | | | | | receptor G1 |
| 9289 | ADGRG1 | Q9Y653 | NM_201524.4 | 4379 | NP_958932.1 | 687 | adhesion G- |
| | | | | | | | protein |
| | | | | | | | coupled |
| | | | | | | | receptor G1 |
| 9289 | ADGRG1 | Q9Y653 | NM_001145771.3 | 4393 | NP_001139243.1 | 693 | adhesion G- |
| | | | | | | | protein |
| | | | | | | | coupled |
| | | | | | | | receptor G1 |
| 9289 | ADGRG1 | Q9Y653 | NM_001145773.3 | 4272 | NP_001139245.1 | 692 | adhesion G- |
| | | | | | | | protein |
| | | | | | | | coupled |
| | | | | | | | receptor G1 |
| 9289 | ADGRG1 | Q9Y653 | NM_001370429.1 | 4275 | NP_001357358.1 | 693 | adhesion G- |
| | | | | | | | protein |
| | | | | | | | coupled |
| | | | | | | | receptor G1 |
| 9289 | ADGRG1 | Q9Y653 | NM_201525.4 | 4257 | NP_958933.1 | 687 | adhesion G- |
| | | | | | | | protein |
| | | | | | | | coupled |
| | | | | | | | receptor G1 |
| 9289 | ADGRG1 | Q9Y653 | NM_001370454.1 | 4225 | NP_001357383.1 | 512 | adhesion G- |
| | | | | | | | protein |
| | | | | | | | coupled |
| | | | | | | | receptor G1 |
| 9289 | ADGRG1 | Q9Y653 | XM_005256248.2 | 3910 | XP_005256305.1 | 693 | adhesion G- |
| | | | | | | | protein |
| | | | | | | | coupled |
| | | | | | | | receptor G1 |
| 9289 | ADGRG1 | Q9Y653 | XM_005256239.2 | 3961 | XP_005256296.1 | 698 | adhesion G- |
| | | | | | | | protein |
| | | | | | | | coupled |
| | | | | | | | receptor G1 |
| 9289 | ADGRG1 | Q9Y653 | XM_005256246.2 | 3946 | XP_005256303.1 | 693 | adhesion G- |
| | | | | | | | protein |
| | | | | | | | coupled |
| | | | | | | | receptor G1 |
| 9289 | ADGRG1 | Q9Y653 | XM_005256244.3 | 3835 | XP_005256301.1 | 698 | adhesion G- |
| | | | | | | | protein |
| | | | | | | | coupled |
| | | | | | | | receptor G1 |
| 9289 | ADGRG1 | Q9Y653 | XM_011523461.2 | 3950 | XP_011521763.1 | 698 | adhesion G- |
| | | | | | | | protein |
| | | | | | | | coupled |
| | | | | | | | receptor G1 |
| 9289 | ADGRG1 | Q9Y653 | XM_011523464.2 | 3935 | XP_011521766.1 | 693 | adhesion G- |
| | | | | | | | protein |
| | | | | | | | coupled |
| | | | | | | | receptor G1 |
| 9289 | ADGRG1 | Q9Y653 | NM_001145774.3 | 4381 | NP_001139246.1 | 687 | adhesion G- |
| | | | | | | | protein |
| | | | | | | | coupled |
| | | | | | | | receptor G1 |
| 9289 | ADGRG1 | Q9Y653 | NM_001290144.2 | 4220 | NP_001277073.1 | 512 | adhesion G- |
| | | | | | | | protein |
| | | | | | | | coupled |
| | | | | | | | receptor G1 |
| 9289 | ADGRG1 | Q9Y653 | XM_011523466.2 | 3920 | XP_011521768.1 | 693 | adhesion G- |
| | | | | | | | protein |
| | | | | | | | coupled |
| | | | | | | | receptor G1 |
| 9289 | ADGRG1 | Q9Y653 | XM_011523468.2 | 3669 | XP_011521770.1 | 693 | adhesion G- |
| | | | | | | | protein |
| | | | | | | | coupled |
| | | | | | | | receptor G1 |
| 9289 | ADGRG1 | Q9Y653 | XM_011523462.2 | 3765 | XP_011521764.1 | 698 | adhesion G- |
| | | | | | | | protein |
| | | | | | | | coupled |
| | | | | | | | receptor G1 |
| 9289 | ADGRG1 | Q9Y653 | XM_005256241.4 | 3686 | XP_005256298.1 | 698 | adhesion G- |
| | | | | | | | protein |
| | | | | | | | coupled |
| | | | | | | | receptor G1 |
| 9289 | ADGRG1 | Q9Y653 | XM_005256252.2 | 3671 | XP_005256309.1 | 693 | adhesion G- |
| | | | | | | | protein |
| | | | | | | | coupled |
| | | | | | | | receptor G1 |
| 9289 | ADGRG1 | Q9Y653 | XM_011523467.2 | 3667 | XP_011521769.1 | 693 | adhesion G- |
| | | | | | | | protein |
| | | | | | | | coupled |
| | | | | | | | receptor G1 |
| 9289 | ADGRG1 | Q9Y653 | XM_006721346.2 | 3613 | XP_006721409.1 | 693 | adhesion G- |
| | | | | | | | protein |
| | | | | | | | coupled |
| | | | | | | | receptor G1 |
| 933 | CD22 | P20273 | NM_001771.4 | 3274 | NP_001762.2 | 847 | B-cell receptor |
| | | | | | | | CD22 |
| 933 | CD22 | P20273 | NM_001185100.2 | 3155 | NP_001172029.1 | 751 | B-cell receptor |
| | | | | | | | CD22 |
| 933 | CD22 | P20273 | NM_001185099.2 | 3010 | NP_001172028.1 | 759 | B-cell receptor |
| | | | | | | | CD22 |
| 933 | CD22 | P20273 | NM_001185101.2 | 2743 | NP_001172030.1 | 670 | B-cell receptor |
| | | | | | | | CD22 |
| 933 | CD22 | P20273 | NM_001278417.2 | 3099 | NP_001265346.1 | 675 | B-cell receptor |
| | | | | | | | CD22 |
| 939 | CD27 | P26842 | XM_017020232.1 | 1591 | XP_016875721.1 | 405 | CD27 antigen |
| 939 | CD27 | P26842 | XM_017020234.1 | 1450 | XP_016875723.1 | 358 | CD27 antigen |
| 939 | CD27 | P26842 | NM_001242.5 | 1245 | NP_001233.2 | 260 | |
| 939 | CD27 | P26842 | XM_017020233.2 | 1746 | XP_016875722.1 | 359 | CD27 antigen |
| 939 | CD27 | P26842 | XM_011521042.3 | 1482 | XP_011519344.1 | 214 | CD27 antigen |
| 940 | CD28 | P10747 | XM_011512194.2 | 5564 | XP_011510496.1 | 234 | T-cell-specific |
| | | | | | | | surface |
| | | | | | | | glycoprotein |
| | | | | | | | CD28 |
| 940 | CD28 | P10747 | XM_011512195.3 | 5307 | XP_011510497.1 | 150 | T-cell-specific |
| | | | | | | | surface |
| | | | | | | | glycoprotein |
| | | | | | | | CD28 |
| 940 | CD28 | P10747 | XM_011512197.2 | 5328 | XP_011510499.1 | 136 | T-cell-specific |
| | | | | | | | surface |
| | | | | | | | glycoprotein |
| | | | | | | | CD28 |
| 940 | CD28 | P10747 | NM_006139.4 | 4721 | NP_006130.1 | 220 | T-cell-specific |
| | | | | | | | surface |
| | | | | | | | glycoprotein |
| | | | | | | | CD28 |
| 940 | CD28 | P10747 | NM_001243077.2 | 4430 | NP_001230006.1 | 123 | T-cell-specific |
| | | | | | | | surface |
| | | | | | | | glycoprotein |
| | | | | | | | CD28 |
| 940 | CD28 | P10747 | NM_001243078.2 | 4364 | NP_001230007.1 | 101 | T-cell-specific |
| | | | | | | | surface |
| | | | | | | | glycoprotein |
| | | | | | | | CD28 |
| 9289 | ADGRG1 | Q9Y653 | XM_024451787.1 | 2186 | XP_024307555.1 | 316 | zinc finger |
| | | | | | | | protein 101 |
| 94039 | ZNF101 | Q8IZC7 | NM_001300949.2 | 4565 | NP_001287878.1 | 316 | zinc finger |
| | | | | | | | protein 101 |
| 94039 | ZNF101 | Q8IZC7 | NM_033204.4 | 4642 | NP_149981.2 | 436 | zinc finger |
| | | | | | | | protein 101 |
| 94039 | ZNF101 | Q8IZC7 | XM_024451786.1 | 2057 | XP_024307554.1 | 316 | zinc finger |
| | | | | | | | protein 101 |
| 94039 | ZNF101 | Q8IZC7 | XM_024451785.1 | 2156 | XP_024307553.1 | 316 | zinc finger |
| | | | | | | | protein 101 |
| 94120 | SYTL3 | Q4VX76 | XM_017011498.2 | 3761 | XP_016866987.1 | 313 | synaptotagmin- |
| | | | | | | | like protein 3 |
| 94120 | SYTL3 | Q4VX76 | XM_017011496.1 | 4597 | XP_016866985.1 | 404 | synaptotagmin- |
| | | | | | | | like protein 3 |
| 94120 | SYTL3 | Q4VX76 | XM_006715605.3 | 4508 | XP_006715668.1 | 610 | synaptotagmin- |
| | | | | | | | like protein 3 |
| 94120 | SYTL3 | Q4VX76 | XM_006715606.3 | 4429 | XP_006715669.1 | 610 | synaptotagmin- |
| | | | | | | | like protein 3 |
| 94120 | SYTL3 | Q4VX76 | XM_011536254.2 | 4197 | XP_011534556.1 | 610 | synaptotagmin- |
| | | | | | | | like protein 3 |
| 94120 | SYTL3 | Q4VX76 | XM_006715611.3 | 4031 | XP_006715674.1 | 404 | synaptotagmin- |
| | | | | | | | like protein 3 |
| 94120 | SYTL3 | Q4VX76 | XM_017011495.1 | 2379 | XP_016866984.1 | 438 | synaptotagmin- |
| | | | | | | | like protein 3 |
| 94120 | SYTL3 | Q4VX76 | XM_017011499.1 | 2084 | XP_016866988.1 | 308 | synaptotagmin- |
| | | | | | | | like protein 3 |
| 94120 | SYTL3 | Q4VX76 | XM_005267215.4 | 4416 | XP_005267272.1 | 610 | synaptotagmin- |
| | | | | | | | like protein 3 |
| 94120 | SYTL3 | Q4VX76 | XM_024446588.1 | 4397 | XP_024302356.1 | 610 | synaptotagmin- |
| | | | | | | | like protein 3 |
| 94120 | SYTL3 | Q4VX76 | XM_017011497.1 | 3934 | XP_016866986.1 | 404 | synaptotagmin- |
| | | | | | | | like protein 3 |
| 94120 | SYTL3 | Q4VX76 | XM_005267218.4 | 3727 | XP_005267275.1 | 404 | synaptotagmin- |
| | | | | | | | like protein 3 |
| 94120 | SYTL3 | Q4VX76 | NM_001242394.2 | 2851 | NP_001229323.1 | 610 | synaptotagmin- |
| | | | | | | | like protein 3 |
| 94120 | SYTL3 | Q4VX76 | NM_001242395.2 | 2647 | NP_001229324.1 | 542 | synaptotagmin- |
| | | | | | | | like protein 3 |
| 94120 | SYTL3 | Q4VX76 | NM_001242384.2 | 2459 | NP_001229313.1 | 610 | synaptotagmin- |
| | | | | | | | like protein 3 |
| 94120 | SYTL3 | Q4VX76 | NM_001009991.4 | 2239 | NP_001009991.2 | 542 | synaptotagmin- |
| | | | | | | | like protein 3 |
| 94120 | SYTL3 | Q4VX76 | NM_001318745.2 | 2669 | NP_001305674.1 | 404 | synaptotagmin- |
| | | | | | | | like protein 3 |
| 94120 | SYTL3 | Q4VX76 | XM_005267222.4 | 2998 | XP_005267279.1 | 404 | synaptotagmin- |
| | | | | | | | like protein 3 |
| 94120 | SYTL3 | Q4VX76 | XM_011536255.2 | 3253 | XP_011534557.1 | 409 | synaptotagmin- |
| | | | | | | | like protein 3 |
| 9840 | TESPA1 | A2RU30 | NR_147063.2 | 4220 | | | |
| 9840 | TESPA1 | A2RU30 | NR_147062.2 | 4393 | | | |
| 9840 | TESPA1 | A2RU30 | NR_147064.2 | 4209 | | | |
| 9840 | TESPA1 | A2RU30 | XR_001748928.1 | 5597 | | | |
| 9840 | TESPA1 | A2RU30 | XR_001748931.1 | 3992 | | | |
| 9840 | TESPA1 | A2RU30 | NR_147065.1 | 3668 | | | |
| 9840 | TESPA1 | A2RU30 | NR_147069.1 | 3617 | | | |
| 9840 | TESPA1 | A2RU30 | NR_147068.1 | 3660 | | | |
| 9840 | TESPA1 | A2RU30 | NR_147071.1 | 3712 | | | |
| 9840 | TESPA1 | A2RU30 | NR_147066.1 | 3716 | | | |
| 9840 | TESPA1 | A2RU30 | NR_147070.1 | 3755 | | | |
| 9840 | TESPA1 | A2RU30 | NR_147067.1 | 3759 | | | |
| 9840 | TESPA1 | A2RU30 | NR_147073.1 | 3847 | | | |
| 9840 | TESPA1 | A2RU30 | NR_147072.1 | 3837 | | | |
| 9840 | TESPA1 | A2RU30 | NM_001136030.3 | 4166 | NP_001129502.1 | 521 | protein |
| | | | | | | | TESPA1 |
| 9840 | TESPA1 | A2RU30 | NM_001351149.2 | 4339 | NP_001338078.1 | 521 | protein |
| | | | | | | | TESPA1 |
| 9840 | TESPA1 | A2RU30 | NM_001098815.3 | 4146 | NP_001092285.1 | 521 | protein |
| | | | | | | | TESPA1 |
| 9840 | TESPA1 | A2RU30 | XM_011539037.3 | 8598 | XP_011537339.1 | 521 | protein |
| | | | | | | | TESPA1 |
| 9840 | TESPA1 | A2RU30 | XM_005269247.2 | 3972 | XP_005269304.1 | 383 | protein |
| | | | | | | | TESPA1 |
| 9840 | TESPA1 | A2RU30 | XM_024449286.1 | 4128 | XP_024305054.1 | 383 | protein |
| | | | | | | | TESPA1 |
| 9840 | TESPA1 | A2RU30 | XM_017020262.1 | 5541 | XP_016875751.1 | 521 | protein |
| | | | | | | | TESPA1 |
| 9840 | TESPA1 | A2RU30 | XM_017020263.1 | 5447 | XP_016875752.1 | 521 | protein |
| | | | | | | | TESPA1 |
| 9840 | TESPA1 | A2RU30 | XM_006719715.3 | 4478 | XP_006719778.1 | 521 | protein |
| | | | | | | | TESPA1 |
| 9840 | TESPA1 | A2RU30 | XM_011539035.2 | 4491 | XP_011537337.1 | 521 | protein |
| | | | | | | | TESPA1 |
| 9840 | TESPA1 | A2RU30 | NM_001261844.1 | 3614 | NP_001248773.1 | 383 | protein |
| | | | | | | | TESPA1 |
| 9840 | TESPA1 | A2RU30 | NM_001351150.1 | 3606 | NP_001338079.1 | 312 | protein |
| | | | | | | | TESPA1 |
| 9840 | TESPA1 | A2RU30 | NM_001351155.1 | 3658 | NP_001338084.1 | 268 | protein |
| | | | | | | | TESPA1 |
| 9840 | TESPA1 | A2RU30 | NM_001351153.1 | 3662 | NP_001338082.1 | 268 | protein |
| | | | | | | | TESPA1 |
| 9840 | TESPA1 | A2RU30 | NM_001351154.1 | 3701 | NP_001338083.1 | 268 | protein |
| | | | | | | | TESPA1 |
| 9840 | TESPA1 | A2RU30 | NM_014796.2 | 3705 | NP_055611.1 | 383 | protein |
| | | | | | | | TESPA1 |
| 9840 | TESPA1 | A2RU30 | NM_001351152.1 | 3779 | NP_001338081.1 | 268 | protein |
| | | | | | | | TESPA1 |
| 9840 | TESPA1 | A2RU30 | NM_001351151.1 | 3783 | NP_001338080.1 | 268 | protein |
| | | | | | | | TESPA1 |
| 9840 | TESPA1 | A2RU30 | NM_001351148.1 | 3826 | NP_001338077.1 | 383 | protein |
| | | | | | | | TESPA1 |

The expression level of the above mentioned genes in blood cells of the subject can be determined on the RNA level or the protein level as further described herein below.

Isolation, extraction or derivation of RNA may be carried out by any suitable method. Isolating RNA from a biological sample generally includes treating a biological sample in such a manner that the RNA present in the sample is extracted and made available for analysis. Any isolation method that results in extracted RNA may be used in the practice of the present invention. It will be understood that the particular method used to extract RNA will depend on the nature of the source.

Methods of RNA extraction are well-known in the art and further described herein under.

Phenol based extraction methods: These single-step RNA isolation methods based on Guanidine isothiocyanate (GITC)/phenol/chloroform extraction require much less time than traditional methods (e.g. CsCl₂ ultracentrifugation). Many commercial reagents (e.g. Trizol, RNAzol, RNAWIZ) are based on this principle. The entire procedure can be completed within an hour to produce high yields of total RNA.

Silica gel-based purification methods: RNeasy is a purification kit marketed by Qiagen. It uses a silica gel-based membrane in a spin-column to selectively bind RNA larger than 200 bases. The method is quick and does not involve the use of phenol.

Oligo-dT based affinity purification of mRNA: Due to the low abundance of mRNA in the total pool of cellular RNA, reducing the amount of rRNA and tRNA in a total RNA preparation greatly increases the relative amount of mRNA. The use of oligo-dT affinity chromatography to selectively enrich poly (A)+ RNA has been practiced for over 20 years. The result of the preparation is an enriched mRNA population that has minimal rRNA or other small RNA contamination. mRNA enrichment is essential for construction of cDNA libraries and other applications where intact mRNA is highly desirable. The original method utilized oligo-dT conjugated resin column chromatography and can be time consuming. Recently more convenient formats such as spin-column and magnetic bead based reagent kits have become available.

The sample may also be processed prior to carrying out the diagnostic methods of the present invention. Processing of the sample may involve one or more of: filtration, distillation, centrifugation, extraction, concentration, dilution, purification, inactivation of interfering components, addition of reagents, and the like.

In another embodiment, the sample of this aspect of the present invention comprises cDNA.

Methods of Detecting the Expression Level of the Genes of the RNA Level

Northern Blot analysis: This method involves the detection of a particular RNA in a mixture of RNAs. An RNA sample is denatured by treatment with an agent (e.g., formaldehyde) that prevents hydrogen bonding between base pairs, ensuring that all the RNA molecules have an unfolded, linear conformation. The individual RNA molecules are then separated according to size by gel electrophoresis and transferred to a nitrocellulose or a nylon-based membrane to which the denatured RNAs adhere. The membrane is then exposed to labeled DNA probes. Probes may be labeled using radio-isotopes or enzyme linked nucleotides. Detection may be using autoradiography, colorimetric reaction or chemiluminescence. This method allows both quantitation of an amount of particular RNA molecules and determination of its identity by a relative position on the membrane which is indicative of a migration distance in the gel during electrophoresis.

RT-PCR analysis: This method uses PCR amplification of relatively rare RNAs molecules. First, RNA molecules are purified from the cells and converted into complementary DNA (cDNA) using a reverse transcriptase enzyme (such as an MMLV-RT) and primers such as, oligo dT, random hexamers or gene specific primers. Then by applying gene specific primers and Taq DNA polymerase, a PCR amplification reaction is carried out in a PCR machine. Those of skills in the art are capable of selecting the length and sequence of the gene specific primers and the PCR conditions (i.e., annealing temperatures, number of cycles and the like) which are suitable for detecting specific RNA molecules. It will be appreciated that a semi-quantitative RT-PCR reaction can be employed by adjusting the number of PCR cycles and comparing the amplification product to known controls.

RNA in situ hybridization stain: In this method DNA or RNA probes are attached to the RNA molecules present in the cells. Generally, the cells are first fixed to microscopic slides to preserve the cellular structure and to prevent the RNA molecules from being degraded and then are subjected to hybridization buffer containing the labeled probe. The hybridization buffer includes reagents such as formamide and salts (e.g., sodium chloride and sodium citrate) which enable specific hybridization of the DNA or RNA probes with their target mRNA molecules in situ while avoiding non-specific binding of probe. Those of skills in the art are capable of adjusting the hybridization conditions (i.e., temperature, concentration of salts and formamide and the like) to specific probes and types of cells. Following hybridization, any unbound probe is washed off and the bound probe is detected using known methods. For example, if a radio-labeled probe is used, then the slide is subjected to a photographic emulsion which reveals signals generated using radio-labeled probes; if the probe was labeled with an enzyme then the enzyme-specific substrate is added for the formation of a colorimetric reaction; if the probe is labeled using a fluorescent label, then the bound probe is revealed using a fluorescent microscope; if the probe is labeled using a tag (e.g., digoxigenin, biotin, and the like) then the bound probe can be detected following interaction with a tag-specific antibody which can be detected using known methods.

In situ RT-PCR stain: This method is described in Nuovo G J, et al. [Intracellular localization of polymerase chain reaction (PCR)-amplified hepatitis C cDNA. Am J Surg Pathol. 1993, 17: 683-90] and Komminoth P, et al. [Evaluation of methods for hepatitis C virus detection in archival liver biopsies. Comparison of histology, immunohistochemistry, in situ hybridization, reverse transcriptase polymerase chain reaction (RT-PCR) and in situ RT-PCR. Pathol Res Pract. 1994, 190: 1017-25]. Briefly, the RT-PCR reaction is performed on fixed cells by incorporating labeled nucleotides to the PCR reaction. The reaction is carried on using a specific in situ RT-PCR apparatus such as the laser-capture microdissection PixCell I LCM system available from Arcturus Engineering (Mountainview, CA).

DNA microarrays/DNA chips: The expression of thousands of genes may be analyzed simultaneously using DNA microarrays, allowing analysis of the complete transcriptional program of an organism during specific developmental processes or physiological responses. DNA microarrays consist of thousands of individual gene sequences attached to closely packed areas on the surface of a support such as a glass microscope slide. Various methods have been developed for preparing DNA microarrays. In one method, an approximately 1 kilobase segment of the coding region of each gene for analysis is individually PCR amplified. A robotic apparatus is employed to apply each amplified DNA sample to closely spaced zones on the surface of a glass microscope slide, which is subsequently processed by thermal and chemical treatment to bind the DNA sequences to the surface of the support and denature them. Typically, such arrays are about 2×2 cm and contain about individual nucleic acids 6000 spots. In a variant of the technique, multiple DNA oligonucleotides, usually 20 nucleotides in length, are synthesized from an initial nucleotide that is covalently bound to the surface of a support, such that tens of thousands of identical oligonucleotides are synthesized in a small square zone on the surface of the support. Multiple oligonucleotide sequences from a single gene are synthesized in neighboring regions of the slide for analysis of expression of that gene. Hence, thousands of genes can be represented on one glass slide. Such arrays of synthetic oligonucleotides may be referred to in the art as “DNA chips”, as opposed to “DNA microarrays”, as described above [Lodish et al. (eds.). Chapter 7.8: DNA Microarrays: Analyzing Genome-Wide Expression. In: Molecular Cell Biology, 4th ed., W. H. Freeman, New York. (2000)].

Oligonucleotide microarray—In this method oligonucleotide probes capable of specifically hybridizing with the polynucleotides of some embodiments of the invention are attached to a solid surface (e.g., a glass wafer). Each oligonucleotide probe is of approximately nucleic acids in length. To detect the expression pattern of the polynucleotides of some embodiments of the invention in a specific cell sample (e.g., blood cells), RNA is extracted from the cell sample using methods known in the art (using e.g., a TRIZOL solution, Gibco BRL, USA). Hybridization can take place using either labeled oligonucleotide probes (e.g., 5′-biotinylated probes) or labeled fragments of complementary DNA (cDNA) or RNA (cRNA). Briefly, double stranded cDNA is prepared from the RNA using reverse transcriptase (RT) (e.g., Superscript II RT), DNA ligase and DNA polymerase I, all according to manufacturer's instructions (Invitrogen Life Technologies, Frederick, MD, USA). To prepare labeled cRNA, the double stranded cDNA is subjected to an in vitro transcription reaction in the presence of biotinylated nucleotides using e.g., the BioArray High Yield RNA Transcript Labeling Kit (Enzo, Diagnostics, Affymetix Santa Clara CA). For efficient hybridization the labeled cRNA can be fragmented by incubating the RNA in 40 mM Tris Acetate (pH 8.1), 100 mM potassium acetate and 30 mM magnesium acetate for 35 minutes at 94° C. Following hybridization, the microarray is washed and the hybridization signal is scanned using a confocal laser fluorescence scanner which measures fluorescence intensity emitted by the labeled cRNA bound to the probe arrays.

For example, in the Affymetrix microarray (Affymetrix®, Santa Clara, CA) each gene on the array is represented by a series of different oligonucleotide probes, of which, each probe pair consists of a perfect match oligonucleotide and a mismatch oligonucleotide. While the perfect match probe has a sequence exactly complimentary to the particular gene, thus enabling the measurement of the level of expression of the particular gene, the mismatch probe differs from the perfect match probe by a single base substitution at the center base position. The hybridization signal is scanned using the Agilent scanner, and the Microarray Suite software subtracts the non-specific signal resulting from the mismatch probe from the signal resulting from the perfect match probe.

Methods of Detecting Expression and/or Activity of the Genes of the Protein Level Expression and/or activity level of proteins expressed in the blood cells of the subject can be determined using methods known in the arts.

Enzyme linked immunosorbent assay (ELISA): This method involves fixation of a sample (e.g., fixed cells or a proteinaceous solution) containing a protein substrate to a surface such as a well of a microtiter plate. A substrate specific antibody coupled to an enzyme is applied and allowed to bind to the substrate. Presence of the antibody is then detected and quantitated by a colorimetric reaction employing the enzyme coupled to the antibody. Enzymes commonly employed in this method include horseradish peroxidase and alkaline phosphatase. If well calibrated and within the linear range of response, the amount of substrate present in the sample is proportional to the amount of color produced. A substrate standard is generally employed to improve quantitative accuracy.

Western blot: This method involves separation of a substrate from other protein by means of an acrylamide gel followed by transfer of the substrate to a membrane (e.g., nylon or PVDF). Presence of the substrate is then detected by antibodies specific to the substrate, which are in turn detected by antibody binding reagents. Antibody binding reagents may be, for example, protein A, or other antibodies. Antibody binding reagents may be radiolabeled or enzyme linked as described hereinabove. Detection may be by autoradiography, colorimetric reaction or chemiluminescence. This method allows both quantitation of an amount of substrate and determination of its identity by a relative position on the membrane which is indicative of a migration distance in the acrylamide gel during electrophoresis.

Radio-immunoassay (RIA): In one version, this method involves precipitation of the desired protein (i.e., the substrate) with a specific antibody and radiolabeled antibody binding protein (e.g., protein A labeled with I¹²⁵) immobilized on a precipitable carrier such as agarose beads. The number of counts in the precipitated pellet is proportional to the amount of substrate.

In an alternate version of the RIA, a labeled substrate and an unlabelled antibody binding protein are employed. A sample containing an unknown amount of substrate is added in varying amounts. The decrease in precipitated counts from the labeled substrate is proportional to the amount of substrate in the added sample.

Fluorescence activated cell sorting (FACS): This method involves detection of a substrate in situ in cells by substrate specific antibodies. The substrate specific antibodies are linked to fluorophores. Detection is by means of a cell sorting machine which reads the wavelength of light emitted from each cell as it passes through a light beam. This method may employ two or more antibodies simultaneously.

Immunohistochemical analysis: This method involves detection of a substrate in situ in fixed cells by substrate specific antibodies. The substrate specific antibodies may be enzyme linked or linked to fluorophores. Detection is by microscopy and subjective or automatic evaluation. If enzyme linked antibodies are employed, a colorimetric reaction may be required. It will be appreciated that immunohistochemistry is often followed by counterstaining of the cell nuclei using for example Hematoxyline or Giemsa stain.

In situ activity assay: According to this method, a chromogenic substrate is applied on the cells containing an active enzyme and the enzyme catalyzes a reaction in which the substrate is decomposed to produce a chromogenic product visible by a light or a fluorescent microscope.

In vitro activity assays: In these methods the activity of a particular enzyme is measured in a protein mixture extracted from the cells. The activity can be measured in a spectrophotometer well using colorimetric methods or can be measured in a non-denaturing acrylamide gel (i.e., activity gel). Following electrophoresis the gel is soaked in a solution containing a substrate and colorimetric reagents. The resulting stained band corresponds to the enzymatic activity of the protein of interest. If well calibrated and within the linear range of response, the amount of enzyme present in the sample is proportional to the amount of color produced. An enzyme standard is generally employed to improve quantitative accuracy.

As mentioned, the methods described herein are used to ascertain what agent should be selected for the treatment of an immune-related disease in a subject.

In one embodiment, the immune-related disease is an inflammatory bowel disease (e.g. Crohn's disease (CD) or ulcerative colitis (UC).

In another embodiment, the immune-related disorder is a chronic immune-related disorder.

Optionally, the immune-related disorder is selected from the group consisting of inflammatory bowel disease, rheumatoid arthritis, ankylosing spondylitis, psoriasis, psoriatic arthritis and Behçet's disease.

According to a particular embodiment, the immune-related diseases is an autoimmune disease, including, but not limited to cardiovascular diseases, rheumatoid diseases, glandular diseases, gastrointestinal diseases, cutaneous diseases, hepatic diseases, neurological diseases, muscular diseases, nephric diseases, diseases related to reproduction, connective tissue diseases and systemic diseases.

Examples of autoimmune cardiovascular diseases include, but are not limited to atherosclerosis (Matsuura E. et al., Lupus. 1998; 7 Suppl 2:S135), myocardial infarction (Vaarala Lupus. 1998; 7 Suppl 2:S132), thrombosis (Tincani A. et al., Lupus 1998; 7 Suppl 2:S107-9), Wegener's granulomatosis, Takayasu's arteritis, Kawasaki syndrome (Praprotnik S. et al., Wien Klin Wochenschr 2000 Aug. 25; 112 (15-16):660), anti-factor VIII autoimmune disease (Lacroix-Desmazes S. et al., Semin Thromb Hemost. 2000; 26 (2):157), necrotizing small vessel vasculitis, microscopic polyangiitis, Churg and Strauss syndrome, pauci-immune focal necrotizing and crescentic glomerulonephritis (Noel L H. Ann Med Interne (Paris). 2000 May; 151 (3):178), antiphospholipid syndrome (Flamholz R. et al., J Clin Apheresis 1999; 14 (4):171), antibody-induced heart failure (Wallukat G. et al., Am J Cardiol. 1999 Jun. 17; 83 (12A): 75H), thrombocytopenic purpura (Moccia F. Ann Ital Med Int. 1999 April-June; 14 (2):114; Semple J W. et al., Blood 1996 May 15; 87 (10):4245), autoimmune hemolytic anemia (Efremov D G. et al., Leuk Lymphoma 1998 January; 28 (3-4):285; Sallah S. et al., Ann Hematol 1997 March; 74 (3):139), cardiac autoimmunity in Chagas' disease (Cunha-Neto E. et al., J Clin Invest 1996 Oct. 15; 98 (8):1709) and anti-helper T lymphocyte autoimmunity (Caporossi A P. et al., Viral Immunol 1998; 11 (1):9).

Examples of autoimmune rheumatoid diseases include, but are not limited to rheumatoid arthritis (Krenn V. et al., Histol Histopathol 2000 July; 15 (3):791; Tisch R, McDevitt H O. Proc Natl Acad Sci units S A 1994 Jan. 18; 91 (2):437) and ankylosing spondylitis (Jan Voswinkel et al., Arthritis Res 2001; 3 (3): 189).

Examples of autoimmune glandular diseases include, but are not limited to, pancreatic disease, Type I diabetes, thyroid disease, Graves' disease, thyroiditis, spontaneous autoimmune thyroiditis, Hashimoto's thyroiditis, idiopathic myxedema, ovarian autoimmunity, autoimmune anti-sperm infertility, autoimmune prostatitis and Type I autoimmune polyglandular syndrome. diseases include, but are not limited to autoimmune diseases of the pancreas, Type 1 diabetes (Castano L. and Eisenbarth G S. Ann. Rev. Immunol. 8:647; Zimmet P. Diabetes Res Clin Pract 1996 October; 34 Suppl: S125), autoimmune thyroid diseases, Graves' disease (Orgiazzi J. Endocrinol Metab Clin North Am 2000 June; 29 (2):339; Sakata S. et al., Mol Cell Endocrinol 1993 March; 92 (1):77), spontaneous autoimmune thyroiditis (Braley-Mullen H. and Yu S, J Immunol 2000 December (12):7262), Hashimoto's thyroiditis (Toyoda N. et al., Nippon Rinsho 1999 August; 57 (8):1810), idiopathic myxedema (Mitsuma T. Nippon Rinsho. 1999 August; 57 (8):1759), ovarian autoimmunity (Garza K M. et al., J Reprod Immunol 1998 February; 37 (2):87), autoimmune anti-sperm infertility (Diekman A B. et al., Am J Reprod Immunol. 2000 March; 43 (3):134), autoimmune prostatitis (Alexander RB. et al., Urology 1997 December; 50 (6):893) and Type I autoimmune polyglandular syndrome (Hara T. et al., Blood. 1991 Mar. 1; 77 (5):1127).

Examples of autoimmune gastrointestinal diseases include, but are not limited to, chronic inflammatory intestinal diseases (Garcia Herola A. et al., Gastroenterol Hepatol. 2000 January; 23 (1):16), celiac disease (Landau Y E. and Shoenfeld Y. Harefuah 2000 Jan. 16; 138 (2):122), colitis, ileitis and Crohn's disease.

Examples of autoimmune cutaneous diseases include, but are not limited to, autoimmune bullous skin diseases, such as, but are not limited to, pemphigus vulgaris, bullous pemphigoid and pemphigus foliaceus.

Examples of autoimmune hepatic diseases include, but are not limited to, hepatitis, autoimmune chronic active hepatitis (Franco A. et al., Clin Immunol Immunopathol 1990 March; 54 (3):382), primary biliary cirrhosis (Jones D E. Clin Sci (Colch) 1996 November; 91 (5):551; Strassburg C P. et al., Eur J Gastroenterol Hepatol. 1999 June; 11 (6):595) and autoimmune hepatitis (Manns M P. J Hepatol 2000 August; 33 (2):326).

Examples of autoimmune neurological diseases include, but are not limited to, multiple sclerosis (Cross AH. et al., J Neuroimmunol 2001 Jan. 1; 112 (1-2):1), Alzheimer's disease (Oron L. et al., J Neural Transm Suppl. 1997; 49:77), myasthenia gravis (Infante A J. And Kraig E, Int Rev Immunol 1999; 18 (1-2):83; Oshima M. et al., Eur J Immunol 1990 December; 20 (12):2563), neuropathies, motor neuropathies (Kornberg AJ. J Clin Neurosci. 2000 May; 7 (3):191); Guillain-Barre syndrome and autoimmune neuropathies (Kusunoki S. Am J Med Sci. 2000 April; 319 (4):234), myasthenia, Lambert-Eaton myasthenic syndrome (Takamori M. Am J Med Sci. 2000 April; 319 (4):204); paraneoplastic neurological diseases, cerebellar atrophy, paraneoplastic cerebellar atrophy and stiff-man syndrome (Hiemstra HS. et al., Proc Natl Acad Sci units S A 2001 Mar. 27; 98 (7):3988); non-paraneoplastic stiff man syndrome, progressive cerebellar atrophies, encephalitis, Rasmussen's encephalitis, amyotrophic lateral sclerosis, Sydeham chorea, Gilles de la Tourette syndrome and autoimmune polyendocrinopathies (Antoine J C. and Honnorat J. Rev Neurol (Paris) 2000 January; 156 (1):23); dysimmune neuropathies (Nobile-Orazio E. et al., Electroencephalogr Clin Neurophysiol Suppl 1999; 50:419); acquired neuromyotonia, arthrogryposis multiplex congenita (Vincent A. et al., Ann N Y Acad Sci. 1998 May 13; 841:482), neuritis, optic neuritis (Soderstrom M. et al., J Neurol Neurosurg Psychiatry 1994 May; 57 (5):544) and neurodegenerative diseases.

Examples of autoimmune muscular diseases include, but are not limited to, myositis, autoimmune myositis and primary Sjogren's syndrome (Feist E. et al., Int Arch Allergy Immunol 2000 September; 123 (1):92) and smooth muscle autoimmune disease (Zauli D. et al., Biomed Pharmacother 1999 June; 53 (5-6):234).

Examples of autoimmune nephric diseases include, but are not limited to, nephritis and autoimmune interstitial nephritis (Kelly CJ. J Am Soc Nephrol 1990 August; 1 (2):140). Examples of autoimmune diseases related to reproduction include, but are not limited to, repeated fetal loss (Tincani A. et al., Lupus 1998; 7 Suppl 2:S107-9).

Examples of autoimmune connective tissue diseases include, but are not limited to, ear diseases, autoimmune ear diseases (Yoo T J. et al., Cell Immunol 1994 August; 157 (1):249) and autoimmune diseases of the inner ear (Gloddek B. et al., Ann N Y Acad Sci 1997 Dec. 29; 830:266).

Examples of autoimmune systemic diseases include, but are not limited to, systemic lupus erythematosus (Erikson J. et al., Immunol Res 1998; 17 (1-2):49) and systemic sclerosis (Renaudineau Y. et al., Clin Diagn Lab Immunol. 1999 March; 6 (2):156); Chan O T. et al., Immunol Rev 1999 June; 169:107).

According to a particular embodiment, the immune-related disease is not a cardiac disease.

Once a candidate drug agent is selected which lowers the immune age of the subject, the present invention further contemplates treating the subject with that agent.

As used herein the term “about” refers to ±10% The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, C T (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, C A (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

Materials and Methods

Sample collection: The cohort consisted of 24 Crohn's disease (CD) patients who received Infliximab anti-TNF treatment at the gastroenterology department of the Rambam Health Care Campus (RHCC) and met the study inclusion criteria as follows: 1) Adequately documented active luminal CD, as phenotyped by a gastroenterologist with expertise in IBD; 2) Documented decision to initiate full infliximab induction regimen with 5 mg/kg induction dosing (i.e. at weeks 0, 2, 6). Patients that had past exposure to Infliximab, Adalimumab or Vedolizumab, or patients who had active infection including febrile diseases or intra-abdominal or perianal abscess were excluded.

Clinical characteristics of the patients are shown in Table 11.

TABLE 11
Clinical and demographic characteristics of CD cohort.

| | IFX | IFX non- | |
|---|---|---|---|
| | responders | responders | |
| Characteristics | (n = 15) | (n = 9) | p* |
| Gender (M/F), n | 9/6 | 6/3 | 1.0 |
| Age (Years) | 36.1 ± 3.5 | 44.1 ± 7.8 | 0.251 |
| Smoking (Never/past/current), n | 8/2/4 | 5/0/4 | 0.466 |
| BMI (kg/m2) | 22.7 ± 0.7 | 21.5 ± 1.9 | 0.539 |
| Worst bowel involvement | 7/1/7 | 2/1/6 | 0.281 |
| (Montreal_L: | | | |
| TI/colon/ileocolon), n | | | |
| Upper GI involvement (n/y), n | 13/2 | 9/0 | 0.511 |
| Disease behavior in CD in the | 5/5/5 | 2/4/3 | 0.874 |
| last 5 years (Montreal_B: | | | |
| No-No/Stricturing/Penetrating), n | | | |
| Presence of Perianal dis. (n/y), | 12/3 | 4/5 | 0.099 |
| n | | | |
| Other extra-intestine | 10/0/5/0/0 | 7/1/1/0/0 | 0.239 |
| presentations of the dis. | | | |
| (none/skin/Musculoskeletal/ | | | |
| Uveitis/Billiary), n | | | |
| Steroids, n | 7 | 4 | 1.0 |
| Thiopurines, n | 10 | 8 | 0.351 |
| MTX, n | 2 | 0 | 0.511 |
| CRP pre-treatment | 18.7 ± 8.3 | 18.8 ± 9.4 | 0.723 |
| CRP 2 w post first treatment | 2.7 ± 0.7 | 8.8 ± 3.2 | 0.128 |
| CRP 14 w post first treatment | 5.1 ± 1.8 | 28.0 ± 13.6 | 0.012 |
| Albumin pre-treatment | 3.6 ± 0.1 | 3.1 ± 0.3 | 0.173 |
| Albumin 2 w post first treatment | 3.7 ± 0.1 | 3.3 ± 0.3 | 0.064 |
| Albumin 14 w post first treatment | 4.0 ± 0.1 | 3.1 ± 0.2 | <0.001 |
| Clinical response 2 w post first | 0/13/2 | 2/5/1 | 0.141 |
| treatment (none/partial/full), n** | | | |
| Clinical response 14 w post first | 1/3/11 | 2/5/2 | 0.052 |
| treatment (none/partial/full), n** | | | |
| HBI pre-treatment | 2.7 ± 0.7 | 7.3 ± 2.8 | 0.880 |
| HBI 14 w post first treatment | 0.8 ± 0.3 | 5 ± 2.2 | 0.102 |
| IFX 2 w post first treatment | 10.7 ± 1.8 | 8.9 ± 1.9 | 0.609 |
| (ng/μl)**** | | | |
| IFX 14 w post first treatment | 3.3 ± 0.5 | 0.9 ± 0.3 | 0.006 |
| (ng/μl)**** | | | |
| positive ADA 2 w post first | 2/15 | 1/7 | 1.0 |
| treatment (y/n), n | | | |
| positive ADA 14 w post first | 1/14 | 0/8 | 1.0 |
| treatment (y/n), n | | | |

Unless indicated otherwise, mean ± SEM are shown. BMI, body mass index; CRP, C reactive protein; HBI, Harvey-Bradshaw Index; IFX, Infliximab; ATI, Antibodies to IFX; MTX, Methotrexate.
*Fisher exact test for categorical data; Wilcoxon test for continuous measure
**Primary clinical response to Infliximab, defined as clinical improvement according to treating physician: 0-none; 1-partial; 2-full
****Maximum drug levels >20, were considered as 20 ng/μl, for the calculation of mean drug levels and SEM.

Patient samples were obtained at three time points: at baseline, before infliximab treatment, and two and fourteen weeks post first treatment and assayed for gene expression microarray data.

Patient response classification was defined by decision algorithm, as described previously (Gaujoux et al. 2018). Briefly, patients were classified as responders based on clinical remission, which was defined as cessation of diarrhea and abdominal cramping or, in the cases of patients with fistulas, cessation of fistula drainage and complete closure of all draining fistulas at week 14, coupled with a decision of the treating physician to continue IFX therapy at the current dosing and schedule. Patients that were defined as partial responders, classification was determined by the decision algorithm that included the following hierarchical rules: 1) steroid dependency at week fourteen; 2) biomarker dynamics (Calprotectin and CRP) and 3) response according to clinical state at week 26.

Applying the decision algorithm and exclusion criteria, yielded a final study cohort of 15 and 9 responding and non-responding patients, respectively.

Blood transcriptome processing and analysis: Whole blood was preserved in PAXgene Blood RNA tubes (PreAnalytiX). RNA was extracted and assayed using Affymetrix Clariom S chips (Thermo Fisher Scientific). The raw gene array data were processed to obtain a log 2 expression value for each gene probe set using the RMA (robust multichip average) method available in the affy R package. Probe set annotation was performed using affycoretools and clariomshumantranscriptcluster.db packages in R. Data were further adjusted for batch effect using empirical Bayes framework applied by the Combat R package.

Results

This example demonstrates the ability of using an external manipulation to dramatically shift patients' immune age on a cohort of Crohn's disease (CD) patients treated with anti-TNF (Infliximab), where this shift in immune-age was associated with a significant impact on the disease's clinical outcome. Using a high-resolution longitudinal gene expression dataset from peripheral blood samples of 24 CD patients, 15 and 9 responding and non-responding patients, at three time points: pretreatment, 2 and 14 weeks post first treatment, the present inventors evaluated the samples' immune-age score reflecting the samples' position along the high dimensional IMM-AGE trajectory. To quantify the immune age of the CD patients, they used normalized gene expression measurements of the following genes: ABLIM1, AFF3, BACH2, BCL11A, BIRC3, BLNK, BTLA, C11orf31, C6orf48, CCR6, CCR7, CD200, CD22, CD27, CD28, CDCA7L, CHMP7, CR2, DPP4, E2F5, EPHX2, FAIM3, FAM102A, FAM134, FCRL1, FCRL2. HOOK1, HVCN1, IL6ST, IMPDH2, KIAA0748, LEF1, LRRN3, MYC, NELL2, NT5E, P2RX5, PAQR8, PLAG1, PTPRK, RCAN3, SCML1, SLC7A6, SNX9, STRBP, SUSD3, TCF4, TCF7, TCL1A, TCTN1, UXT, VPREB3, ZNF101, ZNF671, CRTC3, STAP1 and HLA-DOB.

The immune-age of the CD patients was estimated by using single-sample GSEA algorithm (Barbie D A et al. Nature 462, 108-112 (2009)). An external IBD (CD and UC) disease specific molecular response axis was used to describe the patients' dynamics by generating a PCA on differential gene expression between active and in-active disease states treated with variety of treatment regimens (using public data, GSE94648).

The results of these analyses demonstrated that the immune age was highly correlated with the disease specific molecular axis. Immune age dynamics comparison demonstrated that individuals who responded positively to anti-TNF treatment shifted back their immune age and presented a significant drop unlike non-responders. Furthermore, a significant reduced immune age score in responders was early detected, already 2 weeks post treatment, suggesting its suitability to early assessment of treatment efficiency and adaptation of patient care. The observed shift in immune age using anti-TNF by responders suggest its manipulation to achieve improved clinical outcome.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. A method of treating an immune related disorder in a subject in need thereof comprising:
(a) contacting blood cells of the subject with the agent;
(b) measuring the expression of at least 20 genes selected from the group consisting of ABLIM1, AFF3, BACH2, BCL11A, BIRC3, BLNK, BTLA, C11orf31, C6orf48, CCR6, CCR7, CD200, CD22, CD27, CD28, CDCA7L, CHMP7, CR2, DPP4, E2F5, EPHX2, FAIM3, FAM102A, FAM134B, FCRL1, FCRL2, HOOK1, HVCN1, IL6ST, IMPDH2, KIAA0748, LEF1, LRRN3, MYC, NELL2, NT5E, P2RX5, PAQR8, PLAG1, PTPRK, RCAN3, SCML1, SLC7A6, SNX9, STRBP, SUSD3, TCF4, TCF7, TCL1A, TCTN1, UXT, VPREB3 and ZNF101 in said blood cells of the subject;
(c) obtaining an immune age value based on the expression of said at least 20 genes;
(d) comparing said immune age value with an immune age value of the subject calculated on the basis of expression of said at least 20 genes in blood cells of the subject, in the absence of said agent, wherein a decrease in the immune age of the subject is indicative that the agent has therapeutic efficacy for treating the immune-related disorder; and
(e) administering the agent to the subject.

2. A method of treating an immune related disorder in a subject in need thereof comprising:
(a) contacting blood cells of the subject with the agent;
(b) measuring the expression of each of the genes BACH2, BCL11A, CHMP7, DPP4 and LRRN3 in said blood cells of the subject;
(c) obtaining an immune age value based on the expression of said at least genes;
(d) comparing said immune age value with an immune age value of the subject calculated on the basis of expression of said genes in blood cells of the subject, in the absence of said agent, wherein a decrease in the immune age of the subject is indicative that the agent has therapeutic efficacy for treating the immune-related disorder; and
(e) administering the agent to the subject.

3. A method of analyzing the therapeutic efficacy of a therapeutic agent for treating an immune-related disorder of a subject:
(a) contacting blood cells of the subject with the agent;
(b) measuring the expression of at least 20 genes selected from the group consisting of ABLIM1, AFF3, BACH2, BCL11A, BIRC3, BLNK, BTLA, C11orf31, C6orf48, CCR6, CCR7, CD200, CD22, CD27, CD28, CDCA7L, CHMP7, CR2, DPP4, E2F5, EPHX2, FAIM3, FAM102A, FAM134B, FCRL1, FCRL2, HOOK1, HVCN1, IL6ST, IMPDH2, KIAA0748, LEF1, LRRN3, MYC, NELL2, NT5E, P2RX5, PAQR8, PLAG1, PTPRK, RCAN3, SCML1, SLC7A6, SNX9, STRBP, SUSD3, TCF4, TCF7, TCL1A, TCTN1, UXT, VPREB3 and ZNF101 in said blood cells of the subject;
(c) obtaining an immune age value based on the expression of said at least genes, wherein said immune age value is determined based on expression of no more than 60 genes; and
(d) comparing said immune age value with an immune age value of the subject calculated on the basis of expression of said at least 20 genes in blood cells of the subject, in the absence of said agent, wherein a decrease in the immune age of the subject is indicative that the agent has therapeutic efficacy for treating the immune-related disorder of the subject.

4. (canceled)

5. The method of claim 1, wherein immune age value is determined based on expression of no more than 150 genes.

6. The method of claim 1, wherein immune age value is determined based on expression of no more than 60 genes.

7-10. (canceled)

11. The method of claim 1, further comprising measuring the expression of at least one additional gene selected from the group consisting of AGPAT4, AKAP2, APBB1, ASCL2, C1orf21, C20orf3, CHST12, CST7, CTSW, EEF1B2, ELLS, FAM113B, FAM129C, FCER2, FCGBP, FCRL6, FGFBP2, FLT3LG, GAL3ST4, GPR56, GPR68, GZMH, HOXC4, ID3, LLGL2, LTB, MMP23A, MOBKL2B, MXRA7, MYO6, NKG7, NOG, NOSIP, PCYOX1L, PLEKHF1, PMEPA1, RNF157, RPL12, RPL24, RPS10, RPS13, RPS5, SAP30, SESN2, SYTL3, TBX21, TGFBR3, TNFRSF25, TSPAN13, TTC28, YPEL1, ZNF154, ZNF563, ZNF772, ZSCAN18, C2orf89, PATL2, TTC38, PRR5L, SGK223, NCRNA00287, IGHM, HLA-DOA and IGHV5-78, wherein the immune age value is based on the expression of said at least one additional gene and said expression of said at least twenty genes.

12. The method of claim 1, wherein said contacting is effected in vivo.

13. The method of claim 1, wherein said measuring is effected ex vivo.

14. The method of claim 1, wherein said measuring is effected no earlier than 1 week following said contacting.

15. The method of claim 1, wherein said measuring is effected no more than 14 weeks following said contacting.

16. The method of claim 1, wherein said blood cells comprise peripheral blood cells.

17. The method of claim 1, wherein the immune-related disorder is a chronic immune-related disorder.

18. The method of claim 1, wherein said immune-related disorder is selected from the group consisting of inflammatory bowel disease, rheumatoid arthritis, ankylosing spondylitis, psoriasis, psoriatic arthritis and Behçet's disease.

19. The method of claim 18, wherein said inflammatory bowel disease is Crohn's disease (CD) or ulcerative colitis (UC).

20. The method of claim 1, wherein said agent is an agent that reduces the amount or activity of tumor necrosis factor alpha (TNF-α).

21. The method of claim 20, wherein said agent is an inhibitory antibody that specifically binds to TNF-α.

22. The method of claim 21, wherein said antibody is selected from the group consisting of infliximab, adalimumab, certolizumab pegol and golimumab.

23. The method of claim 20, wherein said agent is selected from the group consisting of etanercept, thalidomide, lenalidomide, pomalidomide, pentoxifulline, bupriopion, R)-DOI, TCB-2, LSD and LA-SS-Az.
